(19) United States
(12) Patent Application Publication
(10) Pub. No.: US 2023/0374480 A1
(43) Pub. Date: Nov. 23, 2023

(54) CAS12A NICKASES

(71) Applicants: BASF Agricultural Solutions Seed US LLC, Florham Park, NJ (US); Wageningen Universiteit, Wageningen (NL)

(72) Inventors: John VAN DER OOST, Renkum (NL); Ricardo Villegas WARREN, Wageningen (NL); Maartje Janneke LUTEIJN, Wageningen (NL); Raymond Hubert Josèphe STAALS, Ede (NL); Wen Ying WU, Wageningen (NL); David DE VLEESSCHAUWER, Gent Zwijnaarde (BE); Katelijn D'HALLUIN, Gent Zwijnaarde (BE); Frank MEULEWAETER, Gent Zwijnaarde (BE)

(21) Appl. No.: 18/176,874

(22) Filed: Mar. 1, 2023

(30) Foreign Application Priority Data
Mar. 1, 2022 (EP) 22159465.8
Oct. 18, 2022 (EP) 22202125.5

(51) Int. Cl. C12N 9/22; C12N 15/63; C12N 15/10

(52) U.S. Cl. CPC C12N 9/22; C12N 15/63; C12N 15/102; C12N 2310/20

(57) ABSTRACT

The present invention relates to the field of gene genome editing. In particular, it relates to the provision of a Cas12a enzyme having nickase activity, as well as the means and methods for the modification of a genomic locus of interest with a Cas12a enzyme having nickase activity and uses thereof.

### CROSS REFERENCE TO RELATED APPLICATIONS

This application claims priority to European Patent Application No. 22202125.5, filed Oct. 18, 2022, and European Patent Application No. 22159465.8, filed Mar. 1, 2022, the disclosures of which are incorporated by reference in their entireties.

### REFERENCE TO AN ELECTRONIC SEQUENCE LISTING

The contents of the electronic sequence listing (038771-00002-Sequence-Listing.xml; Size: 306,834 bytes; and Date of Creation: Feb. 16, 2023) are herein incorporated by reference in its entirety.

### TECHNICAL FIELD

The present invention relates to the field of gene genome editing. In particular, it relates to the provision of a Cas12a enzyme having nickase activity as well as the means and methods for the modification of a genomic locus of interest with a Cas12a enzyme having nickase activity and uses thereof.

### BACKGROUND

Over the past few years, variants of CRISPR nucleases generating single-strand nicks in DNA rather than double-strand breaks (DSBs) have emerged as versatile tools for targeted gene editing in cells and organisms. Target-specific nicking has mainly been achieved by the Cas9 nickase mutants D10A and H840A (Jinek et al., 2012; Gasiunas et al., 2012). Cas9 D10A cleaves the gRNA-targeting strand, while Cas9 H840A cleaves the non-targeted strand (Jinek et al., 2012; Gasiunas et al., 2012; Cong et al., 2013; Mali et al., 2013).

Since nicks are predominantly repaired via the high-fidelity base excision repair pathway (Dianov and Hubscher, 2013), nickases enable highly specific editing. CRISPR nucleases often trigger unexpected cleavage followed by indel formation at genomic sites that share sequence homology with the target site. Paired nickases, which effectively create DSBs by generating two single-strand breaks in proximity on opposite DNA strands, can be introduced to reduce such off-target activity. In this dual nickase approach, long overhangs are produced on each of the cleaved ends instead of blunt ends. This provides enhanced control over precise gene integration and insertion. Because both nicking enzymes must effectively nick their target DNA, paired nickases have significantly lower off-target effects compared to the double-strand-cleaving Cas system (Ran et al., 2013; Kuscu et al., 2014).

Besides reducing off-target editing, nickases can also be leveraged to boost the efficiency of precision gene editing methods such as homology-directed repair (HDR) and base editing. HDR initiated by double-stranded DNA cleavage is usually accompanied by unwanted insertions and deletions (indels) at on-target and off-target sites (Kosicki et al., 2018; Shin et al., 2017; Tsai et al., 2015; Zhang et al., 2015). Nickases offer an attractive approach to induce high-fidelity HDR without stimulating NHEJ. Base editing similarly allows base substitution at a target site without concurrent indel formation. Since base editors do not normally create a DSB, they minimize the generation of DSB-associated byproducts (Komor et al., 2016; Gaudelli et al., 2017). DNA base editors (BEs) comprise fusions between a catalytically inactive Cas nuclease or nickase and a base-modification enzyme that operates on single-stranded DNA (ssDNA) but not double-stranded DNA (dsDNA). Upon binding to its target locus in DNA, base pairing between the guide RNA and target DNA strand leads to displacement of a small segment of single-stranded DNA in a so-called “R-loop” (Nishimasu et al., 2014).

DNA bases within this single-stranded DNA bubble are modified by the deaminase enzyme. To improve editing efficiency, many base editors have been designed to introduce a nick in the non-edited DNA strand, thereby inducing cells to repair the non-edited strand using the edited strand as a template (Komor et al., 2016; Nishida et al., 2016; Gaudelli et al., 2017).

Importantly, nickases, if suitably adapted, can also fulfil an essential role in the recently developed prime editing technology. Prime editing is a “search-and-replace” genome editing tool that mediates targeted insertions, deletions, all 12 possible base-to-base conversions, and combinations thereof without requiring DSBs or donor templates (Anzalone et al., 2019). Prime editors use a reverse transcriptase fused to an RNA-programmable nickase and a prime editing extended guide RNA to directly copy genetic information from the extension on the pegRNA into the target genomic locus. In this approach, the Cas9 H840A nickase is used to nick the non-target strand to expose a 3-hydroxyl group that primes the reverse transcription of the edit-encoding extension on the pegRNA directly into the target site. Moreover, much like base editors, third-generation prime editors additionally nick the non-edited strand to induce its replacement and further increase editing efficiency (Anzalone et al., 2019). As the skilled person is well aware, pegRNA can be designed and optimized depending on the desired target cell or construct. For example, prime editing in plants is described in Sretenovic and Qi 2021 and optimized prime editing in monocot plants is described in Jin et al., 2022.

Of course, the search for versatile base and prime editors requires both a sound basic functionality of the nickase itself (high specificity, broad PAM targeting range, stability, low off-target and high on-target activity) as well as the proper steric integration of the nickase domain with other domains and spacers between the effector domains etc. so that a proper modular architecture and highly efficient activity on/at a target site in a selected genome can be achieved.

Presently, CRISPR-Cas systems are classified into two classes (Classes 1 and 2) that are subdivided into six types (types I through VI). Class 1 (types I, III and IV) systems use multiple Cas proteins in their CRISPR ribonucleoprotein effector nucleases and Class 2 systems (types II, V and VI) use a single Cas protein (Nishimasu et al., 2017). Besides the CRISPR Cas9 system, the CRISPR Cas12a (or Cpf1) system has emerged as a powerful biotechnological tool for a plethora of genome editing applications.

Cas9 generates blunt-ended DSBs by simultaneously cleaving both DNA strands through the combined activity of two conserved nuclease domains, RuvC and HNH (Jinek et al., 2012; Gasiunas et al., 2012). A Cas9 nickase variant can be generated by alanine substitution of key catalytic residues within these domains: the RuvC mutant D10A produces a nick on the targeting strand while the HNH mutant H840A generates a nick on the non-targeting strand DNA (amino acid numbering of Cas9 from Streptococcus pygenes, SpCas9; Jinek et al., 2012; Gasiunas et al., 2012; Cong et al., 2013; Mali et al., 2013).

Recently, it has been described for plant cells (WO2021122080A1) that introduction of paired nicks strongly improves the efficiency of homology-directed repair, enabling precise introduction of donor DNA sequences into plant genomes by reducing random insertions and/or deletions (Indels). Such nickase-based approaches can greatly reduce screening efforts.

A further approach to improve specific and targeted modifications of DNA are guide RNAs that are covalently linked to donor nucleotides thereby enhancing HDR efficiency (WO2017186550A1). Such fusion nucleic acid molecules could be combined with efficient Cas12a nickases to achieve optimal efficiency and specificity when introducing donor sequences into target genomes.

In contrast to earlier findings with Cas9 nickases, target-specific nicking has not yet been achieved for Cas12a so far, particularly not in relevant crop plants, and there is thus a great need to establish suitable Cas12a-based nickase tools.

Unlike Cas9, Cas12a cleaves both DNA strands sequentially using a single catalytic site located in the RuvC domain, while the Nuc domain plays a role in substrate DNA coordination (Swarts et al., 2017, 2019). This difference in structural organization hampers the design of true nickases of Cas12a in comparison to Cas9, the latter CRISPR nuclease having two distinct domains comprising two individual active domains, HNH and RuvC, catalyzing the cleavage of the target and the non-target strand, respectively.

In the LbCas12a structure, the RuvC active site is formed by the conserved acidic residues Asp832, Glu925, Asp1180, and Arg1138 (Yamano et al., 2017). In vitro cleavage assays showed that the D832A, E925A, and D1180A mutations completely abolish the DNA cleavage activity of LbCas12a, while the R1138A mutant was reported to function as an at least partially active nickase in vitro, as is the case of R1226A AsCas12a (Zetsche et al., 2015; Yamano et al., 2016). As also reported in Yamano et al., 2017, LbCas12a and AsCas12a are structurally and functionally related. In particular, these Cas12a variants both share the overall domain architecture. Another reported nickase variant includes a FnCas12a K1013G/R1014G double mutant which was reported to cut only the target strand (WO 2019/233990).

Yet to date, there is no evidence showing specific nickase activity in vivo of a Cas12a nicking variant and, consequently, there is no generally applicable Cas12a nickase having high and specific nicking activity in vivo in a variety of eukaryotic cells.

Given the central role of nickases in multiple genome editing tools (HDR, base editing, prime editing), development of a Cas12a variant exhibiting efficient DNA nicking in vivo, including in planta, is key to leveraging the full potential of Cas12a for crop genetic improvement, therapeutic applications and applications in food and nutritional sciences.

While CRISPR-Cas applications are very difficult in wheat, one of the most important crop plants worldwide, but difficult to modify genetically, efficient methods for the precise introduction of donor DNA sequences into wheat genomes have recently been developed (WO2021122081A1). Efficient and specific Cas12a nickases may thus also have great potential for improving precise genetic modification in wheat.

Therefore, it was an overarching objective to engineer and identify one or more Cas12a nickase variants through a rational design approach and via a directed evolution approach, said nickases allowing for the in vitro and particularly also in vivo generation of nicks (or pairs of nicks) in chromosomal DNA of a broad range of prokaryotic and also eukaryotic organisms, wherein the Cas12a nickase should have highly specific nickase activity and low off-target activity as well as high flexibility to be used in various genome modification settings, including base editing, prime editing and paired-nickase assays and an overall robustness and stability to provide a broadly applicable genome nicking tool.

### Definitions

Broad spectrum nickase activity as used herein refers to the capability to efficiently generate specific single-strand DNA breaks (nicks), both in vitro and in vivo, and with minimal to no residual nuclease activity, preferably wherein residual nuclease activity in vitro and/or in vivo, preferably in vitro and in vivo, is less than approximately 20%, more preferably less than approximately 15%, even more preferably less than approximately 10%, and most preferably less than approximately 5% of total enzyme activity, wherein the total enzyme activity is the sum of nickase activity and nuclease activity of a given Cas12a enzyme having nickase activity or catalytically active fragment thereof, wherein the nickase activity and nuclease activity of a given Cas12a enzyme having nickase activity or catalytically active fragment thereof are determined and compared with the same detection system and/or method in a suitable cellular and/or in vitro system using suitable and reasonable reaction conditions and further using the same target site(s) under the same conditions within reasonable limits of said cellular and/or in vitro system. The skilled person is well aware of various different suitable methods to determine nickase and nuclease activity of a Cas12a enzyme, including methods disclosed herein. The term “nuclease activity” as used herein refers to endonucleolytic activity wherein one nuclease effector is able to generate a double-strand break, whereas for a nickase—to achieve a double-strand break—two individual nicks (by the same, or by at least two different nickases) are needed. Target strand (TS) nickase activity as used herein refers to nickase activity as described above, wherein at least 90% of the nicking occurs in the target strand. Non-target strand (NTS) nickase activity as used herein refers to nickase activity as described above, wherein at least 90% of the nicking occurs in the non-target strand.

A target site as used herein refers to both strands of a double-stranded DNA, i.e. a target strand—to which a guide RNA anneals—and a complementary non-target strand, wherein the target site is the stretch of DNA for with a guide RNA has suitable complementarity to the target strand, wherein in embodiments, in which at least two compatible guide RNAs are designed to allow a concerted action of one or at least two Cas enzymes, the target site refers to the at least two stretches of DNA for each of which one guide RNA has complementarity to the target strand, and further includes any DNA sequence in between said at least two stretches of DNA (cf. also FIG. 7A), wherein said at least two stretches of DNA for each of which one guide RNA has complementarity may also overlap or may be identical.

“At or near a target site” as used herein refers to the part of DNA that is within the target site or up to 10 bp, up to 20 bp, up to 30 bp, or up to 40 bp next to the target site, including both directions.

A “donor repair template”, or donor template”, or “donor DNA” or simply “donor” refers to a nucleic acid template that may be provided to allow and mediate HDR, which may be used to achieve error free modification of a target locus and/or the introduction of foreign nucleic acid sequences, such as transgenes. The at least one donor repair template may comprise or encode a double- and/or single-stranded nucleic acid sequence. The at least one donor repair template may comprise or encode an RNA and/or DNA sequence. The at least one donor repair template may comprise or encode symmetric or asymmetric homology arms. In certain embodiments, the at least one donor repair template may further comprise at least one chemically modified base and/or backbone, such as a fluorescent marker and/or a phosphothioate modified backbone. The design and use of donor repair templates for various purposes are well known to the skilled person.

The term “disease-state-related target site” as used herein refers to any target site for which a certain allele, variant or mutation actually or potentially causes, influences or may be a risk factor for at least one physical and/or mental disease, ailment, disorder or adverse condition or propensity, or the progression or prognosis thereof. A disease-state-related target site may for example be a target site comprising a missense or nonsense mutation within a protein-coding gene or it may be a target site comprising a variant of a polymorphism, such as a single-nucleotide polymorphism, that correlates may be a risk factor for the development of a certain disease.

The term “guide RNA” may refer to any RNA comprising a Cas-protein-binding region and a targeting region and is capable of guiding a Cas protein to a target nucleotide sequence being sufficiently complementary to the targeting region of the guide RNA as long as the target nucleotide sequence is located next to a PAM sequence suitable for the respective Cas protein. For Cas12a systems, the terms “guide RNA”, “crRNA”, gRNA” or “sgRNA” are used interchangeably. For systems and/or approaches using a two-molecule guide RNA in the natural environment as known in the art, such as a crRNA and a tracrRNA, the term guide RNA refers to both RNA molecules. Once a CRISPR effector system including a Cas enzyme and the cognate guide RNA (crRNA, or crRNA::tracrRNA) is described, the skilled person is thus aware which type of guide RNA is used for which type of Cas enzyme, for instance a Cas12a system uses a single crRNA, whereas a Cas12e system uses a crRNA::tracrRNA duplex similar to a Cas9 system, wherein a crRNA::tracrRNA duplex may however be mimicked by a synthetic single guide RNA molecule. Further, the skilled person is well aware of designing, expressing/synthesizing and adapting guide RNAs for the purposes needed. Particularly, the mutations to (n)Cas12a enzymes and (n)Cas12 orthologs thereof as provided herein will not have an influence on the overall design and the mode of interaction of the cognate guide RNA for a given nCas12a enzyme, or a nCas12 ortholog. In embodiments relating to a prime editor or prime editor complex, the guide RNA may be a pegRNA (prime editing guide RNA), and may further comprise a primer binding site (PBS) and/or a reverse transcriptase template sequence. The design of guide RNAs, including pegRNAs, suitable for various different Cas systems is well known to the skilled person.

“Identity” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned overtheir complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

| | | |
|---|---|---|
| Seq A: | AAGATACTG | length: 9 bases |
| Seq B: | GATCTGA | length: 7 bases |

Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “|” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)⁺100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)⁺100=66.7%; for Seq B being the sequence of the invention (6/8)⁺100=75%.

“Indel” is a term for the random insertion or deletion of bases in the genome of an organism associated with the repair of a DSB by NHEJ. It is classified among small genetic variations, measuring from 1 to 10 000 base pairs in length. As used herein it refers to random insertion or deletion of bases in or in the close vicinity (e.g. less than 1000 bp, 900 bp, 800 bp, 700 bp, 600 bp, 500 bp, 400 bp, 300 bp, 250 bp, 200 bp, 150 bp, 100 bp, 50 bp, 40 bp, 30 bp, 25 bp, 20 bp, 15 bp, 10 bp or 5 bp up and/or downstream) of the target site.

The term in vitro as used herein refers to the state or quality of a method or application or procedure of not being performed inside of a living cell, preferably in a cell-free system. In vitro methods, applications or procedures are typically performed with biological material, such as nucleic acids, polypeptides and the like that have been purified from cells and/or were artificially processed or synthesized, usually in a reaction tube or reaction compartment comprising a suitable buffer system and suitable reaction components.

The term in vivo as used herein refers to the state or quality of a method, application or procedure of comprising the manipulation of at least one living cell (including cells grown in cell culture), such as the introduction of CRISPR components into living cells and potential genomic nicking, double-strand cleavage and/or modification within said cells. In vivo methods, applications or procedures may be followed by in vitro analysis of e.g. purified DNA after cell lysis. In vivo as used herein, therefore, does not necessarily imply that a method is performed within a living organism, the in vivo method can be performed in an in vitro environment, such as in vitro cell culture.

The term ex vivo as used herein refers to the state or quality of a method, application or procedure to be directed at living cells and/or living tissue extracted from an organism, wherein said living cells and/or living tissue may be re-inserted into the organism, from which it was extracted, after the ex vivo method, application or procedure.

The term “offset” as used herein refers to the number of base pairs between the binding sites of two guide RNAs designed to allow concerted action of one or at least two Cas enzymes (cf. FIG. 7A showing an example offset of +5 bp).

### BRIEF DESCRIPTION OF FIGURES

FIG. 1 (FIG. 1) shows an excerpt from an alignment of the full-length sequences of SEQ ID NOs: 1 to 12 generated with CLUSTAL Omega (version 1.2.4) multiple sequence alignment. Particularly, FIG. 1 shows the sequence identified as the “core lid domain” herein highlighted in bold and starting from position L927 and ending at position V942 with respect to LbCas12a (SEQ ID NO:1) as reference sequence, said reference core lid domain sequence being additionally highlighted by underlining. The catalytically active E925 of LbCas12a, fully conserved in all Cas12a orthologs/homologs shown (and in others not shown, for example, FnCas12a from UniProt accession AOQ7Q2), is highlighted by underlining. The following parameters were used for the alignment: Input Parameters: Output guide tree=true; Output distance matrix=false; Dealign input sequences=false; mBed-like clustering guide tree=true; mBed-like clustering iteration=true; Number of iterations=0; Maximum guide tree iterations=−1; Maximum HMM iterations=−1; Output alignment format=clustal_num: Output order=aligned; Sequence Type=protein. Displayed sequences are included in the shown order as SEQ ID NOs: 123 to 134, respectively.

FIG. 2 (FIG. 2) shows a sketch of the LbCas12a domain architecture and a rough 2D model of the approximate protein structure in contact with a crRNA and a target DNA. PI: PAM-interacting domain, BH: bridge helix. The star in the domain overview and in the model drawing represents the approximate position of RuvC lid mutations according to the present invention.

FIG. 3 (FIG. 3) shows a model drawing of the E. coli GFP/RFP detection assay used to analyze in vivo nickase activity (by detecting paired nicking) and nuclease activity. Shown Cas12a vectors symbolize either a Cas12a variant library or one or more specific Cas12a variant(s). “sgRNA1” denotes a sequence encoding a guide RNA suitable for targeting a first target site (“PS-1”) and “sgRNA2” denotes a sequence encoding a guide RNA suitable for targeting a second target site (“PS-2”). “Cas12a” in this figure denotes a Cas12a enzyme having nuclease activity, “nCas12a” in this figure denotes a Cas12a enzyme having nickase activity, “dCas12a” in this figure denotes a Cas12a enzyme being a dead Cas12a, i.e. having neither nickase nor nuclease activity. Only ideal states are shown, Cas12a variants may also exhibit a combination of nickase activity and nuclease activity and/or lowered nickase activity and/or nuclease activity.

FIG. 4 (FIG. 4) shows the results of GFP/RFP detection for selected Cas12a variants. WT: wild type LbCas12a, dLbCas12a: LbCas12a D832A/E925A (mutations relate to reference sequence SEQ ID NO: 1); LbCas12a R1138A, LbCas12a K932G/N933G and LbCas12a S934A/R935G: mutation relates to reference sequence SEQ ID NO: 1; LbCas12a K932G/N933G/S934A/R935G: quadruple lid mutant (SEQ ID NO: 14); RuvC^{L-neg}: negative RuvC Lid mutant (LbCas12a F931 E/K932E/R935D/K937D/K940D, mutation relates to reference sequence SEQ ID NO: 1). Y-Axis shows relative fluorescence intensity, i.e. fluorescence intensity relative to the amount of measured E. coli cells (as determined by the optical density (OD600) of the E. coli culture). Light grey bars depict GFP-derived fluorescence, dark grey bars show RFP-derived fluorescence.

FIG. 5A (FIG. 5A) shows RuvC lid amino acid sequences of Cas12a variants shown in FIG. 5B. Shown Cas12a proteins are: LbCas12a WT (SEQ ID NO: 1), pRV26002 (SEQ ID NO: 23), pRV26004 (SEQ ID NO: 16), pRV26006 (SEQ ID NO: 20), pRV26008 (SEQ ID NO: 21), pRV26010 (SEQ ID NO: 19), pRV26180 (SEQ ID NO: 22), pRV26182 (SEQ ID NO: 18), pRV26184 (SEQ ID NO: 17). Displayed sequences are included in the shown order as SEQ ID NOs: 135 to 143, respectively.

FIG. 5B (FIG. 5B) shows the results of GFP/RFP detection for selected Cas12a variants. Shown Cas12a proteins are: WT (SEQ ID NO: 1), dLbCas12a (LbCas12a D832A/E925A, mutations relate to reference sequence SEQ ID NO: 1) pRV26002 (SEQ ID NO: 23), pRV26004 (SEQ ID NO: 16), pRV26006 (SEQ ID NO: 20), pRV26008 (SEQ ID NO: 21), pRV26010 (SEQ ID NO: 19), pRV26180 (SEQ ID NO: 22), pRV26182 (SEQ ID NO: 18), pRV26184 (SEQ ID NO: 17). Light grey bars depict GFP-derived fluorescence, dark grey bars show RFP-derived fluorescence.

FIG. 5C (FIG. 5C) shows the results of GFP/RFP detection for selected Cas12a variants. Shown Cas12a proteins are: WT (SEQ ID NO: 1), dLbCas12a (LbCas12a D832A/E925A, mutations relate to reference sequence SEQ ID NO: 1) Lid1.2 (SEQ ID NO: 24), Lid2.3 (SEQ ID NO: 25), Lid2.4 (SEQ ID NO: 26). Light grey bars depict GFP-derived fluorescence, dark grey bars show RFP-derived fluorescence.

FIG. 5D (FIG. 5D) shows the amino acid sequence within the mutagenized RuvC lid region of selected LbCas12a nickase variants the Column “Sequence” shows amino acids at position 930 to 933 of the respective SEQ ID NO. Additionally, the respective sub-sequences are additionally provided with SEQ ID NOs. 107 to 113).

FIG. 5E (FIG. 5E) shows the results of GFP/RFP detection for selected Cas12a variants. Shown Cas12a proteins are: LbCas12a wt (SEQ ID NO: 1), LbCas12a dead (LbCas12a D832A/E925A, mutations relate to reference sequence SEQ ID NO: 1) Lid2.3 (SEQ ID NO: 15), Lid4.1 (SEQ ID NO: 100), Lid4.2 (SEQ ID NO: 101), Lid4.3 (SEQ ID NO: 102), Lid4.4 (SEQ ID NO: 103), Lid4.5 (SEQ ID NO: 104), Lid4.6 (SEQ ID NO: 105), Lid4.7 (SEQ ID NO: 106). Light grey bars depict GFP-derived fluorescence, dark grey bars show RFP-derived fluorescence.

FIG. 6A (FIG. 6A) shows RuvC lid amino acid sequences of Cas12a variants shown in FIG. 6B. Displayed sequences are included in the shown order as SEQ ID NOs: 135, 144 and 145, respectively.

FIG. 6B (FIG. 6B) shows the results of an in vitro plasmid cleavage assay. Shown Cas12a proteins are: LbCas12a WT (SEQ ID NO: 1), dLbCas12a (LbCas12a D832A/E925A, mutations relate to reference sequence SEQ ID NO: 1), pRV26004 (SEQ ID NO: 16), RuVC_{L del1} (lid deletion variant 1, SEQ ID NO: 15). pT: Target plasmid, a plasmid comprising a target site for the used cRNA; pUC19: control plasmid without target site for the used cRNA; EcoRI and NB.BvCI refer to respective restriction endonuclease and nickase, respectively; N: nicked; L: linear; S: supercoiled.

FIG. 6C (FIG. 6C) shows a method for analysis of nicked target DNA by Sanger run-off sequencing. Nicked substrates resulting from in vitro digestion of target plasmids are extracted from agarose gels, purified and subjected to Sanger sequencing using primers targeting either the top or bottom strand. Shown Cas12a proteins are: LbCas12a WT (SEQ ID NO: 1), LbCas12a dead (LbCas12a D832A/E925A, mutations relate to reference sequence SEQ ID NO: 1), FnCas12a K969P/D970P (mutations relate to reference sequence SEQ ID NO: 3), LbCas12a R1138A (mutations relate to reference sequence SEQ ID NO: 1), RuvC^{L-del1} (lid deletion variant 1, SEQ ID NO: 15). pT: Target plasmid, a plasmid comprising a target site for the used cRNA; pUC19: control plasmid without target site for the used cRNA; EcoRI and Nt.BbvCI refer to respective restriction endonuclease and nickase, respectively; N: nicked; L: linear; S: supercoiled

FIG. 6D (FIG. 6D) shows a model drawing of the dsDNA substrates used in an in vitro fluorescent nickase activity assay. The DNA substrates are labelled with Cy5 on the target strand and with Cy3 on the non-target strand. A shift in the position of the fluorescent DNA bands indicates that the strand was cleaved

FIG. 6E (FIG. 6E) shows the results of an in vitro fluorescent nickase assay. Shown Cas12a proteins are: LbCas12a WT (SEQ ID NO: 1), dLbCas12a (LbCas12a D832A/E925A, mutations relate to reference sequence SEQ ID NO: 1), RuVC^{L del1} (lid deletion variant 1, SEQ ID NO: 15), RuvC^{L-del1 C931E} (lid deletion variant 1+C931E, SEQ ID NO: 56). Non-digested: control reaction comprising fluorescently-labeled DNA substrates only; EcoRI and Nt.BvCI ref42er to respective restriction endonuclease and nickase, respectively; ‘−’ and ‘+’ indicate the absence and presence of selected Cas12a proteins in the nicking reaction. Different incubation times were tested for nicking reactions with the RuvC^{L-del1 C931E} mutant, all other reactions were incubated for 1h at 37° C.

FIG. 7A (FIG. 7A) shows an example set up for paired nicking with an offset of +5 bp. sgRNA3 and sgRNA9 denote two different guide RNAs. Italic letters indicate the nucleic acid sequence having complementarity to the respective guide RNA, i.e. the guide RNA binding sites on the respective target strand. Bold letters indicate the nucleic acid sequence (on the respective non-target strand) corresponding to the sequence in the targeting region of the respective guide RNA. The gray box indicates the target site in this exemplary paired nickase set up. This set up was used in exemplary paired nickase assay shown in FIG. 7B. Note that this exemplary set up was designed for Cas9-mediated nicking and therefore comprises PAMs suitable for a Cas9 protein. For Cas12a paired nickase strategies, PAMs suitable for the respective Cas12a protein must be chosen. Top DNA strand: SEQ ID NO: 34; bottom DNA strand: SEQ ID NO: 35.

FIG. 7B (FIG. 7B) shows exemplary results of the in vitro TXTL paired nicking assay, with a Cas9 D10A nickase and two different guide RNAs (see FIG. 7A) targeting the GFP-encoding sequence. GFP fluorescence overtime is shown in light gray for the individual sample and in dark gray for a control in which the GFP-encoding sequence is not targeted. Cas9-sg3: Cas9 nuclease with the first guide RNA (sg3: sgRNA3); nCas9 D10A-sg3: Cas9 D10A nickase with the first guide RNA; nCas9 D10A-sg9: Cas9 D10A nickase with the second guide RNA (sg9: sgRNA9); nCas9 D10A-sg3+sg9: Cas9 D10A nickase with the first and the second guide RNA.

FIG. 8A (FIG. 8A) shows an analysis of editing outcomes at the OsAAT target site in rice protoplasts transfected with Cas12a nickase candidates. The Y-Axis shows the percentage of sequencing reads with indels. Shown Cas12a proteins are LbCas12a (SEQ ID NO: 1), LbCas12a R1138A (mutation relates to reference sequence SEQ ID NO: 1), LbCas12a K932G/N933G (mutation relates to reference sequence SEQ ID NO: 1), LbCas12a K932G/N933G/S934A/R935G: quadruple lid mutant (SEQ ID NO: 14).

FIG. 8B (FIG. 8B) shows an analysis of editing outcomes at the OsAAT target site in rice protoplasts transfected with Cas12a nickase candidates. The Y-Axis shows the percentage of sequencing reads with base substitutions. Shown Cas12a proteins are LbCas12a (SEQ ID NO: 1), LbCas12a R1138A (mutation relates to reference sequence SEQ ID NO: 1), LbCas12a K932G/N933G (mutation relates to reference sequence SEQ ID NO: 1), LbCas12a K932G/N933G/S934A/R935G: quadruple lid mutant (SEQ ID NO: 14).

FIG. 8C (FIG. 8C) shows a comparative representation of the data shown in FIG. 8A and in FIG. 8B. Column I shows the nuclease activity in percent of the wild type LbCas12a (WT), column II shows the percentage of edited reads with indels and column III shows the percentage of edited reads with base substitutions.

FIG. 9A (FIG. 9A) shows the concept of the GFP/dsRed paired nicking assay. The GFP-encoding sequence is targeted by two guide RNAs, while the dsRED-encoding sequence is targeted by one. “Cas12a” in this figure denotes a Cas12a enzyme having nuclease activity, “nCas12a” in this figure denotes a Cas12a enzyme having nickase activity, “dCas12a” in this figure denotes a Cas12a enzyme being a dead Cas12a, i.e. having neither nickase nor nuclease activity. Only ideal states are shown, Cas12a variants may also have a combination of nickase activity and nuclease activity and/or lowered nickase activity and/or nuclease activity.

FIG. 9B (FIG. 9B) shows example fluorescence microscopy images of in planta GFP/dsRed paired nicking analysis. Rice protoplasts were transfected with either no Cas protein (Ctrl.); Wild type LbCas12a (SEQ ID NO:1), dead LbCas12a D893A (mutation relates to reference sequence of SEQ ID NO:1); or LbCas12a K932G/N933G/S934A/R935G (SEQ ID: NO 14).

FIG. 10A (FIG. 10A) shows the results of different LbCas12a base editor constructs at the OsAAT target site in rice protoplasts. Y-axis shows the percentage of reads with base edits. LbCas12a-D832A and LbCas12a-K932G/N933G: mutations relate to reference sequence SEQ ID NO:1. LbCas12a K932G/N933G/S934A/R935G: SEQ ID: NO 14.

FIG. 10B (FIG. 10B) shows the results of different LbCas12a base editor constructs at the OsAAT target site in rice protoplasts. Y-axis shows the percentage of reads with indels. LbCas12a-D832A and LbCas12a-K932G/N933G: mutations relate to reference sequence SEQ ID NO:1. LbCas12a K932G/N933G/S934A/R935G: SEQ ID: NO 14.

FIG. 11 (FIG. 11) shows an analysis of editing outcomes at the OsAAT target site in rice protoplasts transfected with Cas12a nickase candidates. The Y-Axis shows the percentage of sequencing reads with indels. Shown Cas12a proteins are LbCas12a (SEQ ID NO: 1), LbCas12a-RuvC lid deletion (SEQ ID NO: 15) and LbCas12a-RuvC lid deletion/C931E (SEQ ID NO: 56).

FIG. 12A (FIG. 12A) shows the results of different LbCas12a base editor constructs at the OsAAT target site in rice protoplasts. The base editors contain either LbCas12a-D832A (mutation relates to reference sequence SEQ ID NO: 1), LbCas12a-RuvC lid deletion (SEQ ID NO: 15) or LbCas12a-RuvC lid deletion/C931 E (SEQ ID NO: 56) as the Cas moiety. Y-axis shows the base editing efficiency expressed relative to that shown by the LbCas12a-D832A editor.

FIG. 12B (FIG. 12B) shows the results of different LbCas12a base editor constructs at the BnFAD2 target site in oilseed rape protoplasts. The base editors contain either LbCas12a-D832A (mutation relates to reference sequence SEQ ID NO: 1), LbCas12a-RuvC lid deletion (SEQ ID NO: 15) or LbCas12a-RuvC lid deletion/C931E (SEQ ID NO: 56) as the Cas moiety. Y-axis shows the base editing efficiency expressed relative to that shown by the LbCas12a-D832A editor.

FIG. 13 (FIG. 13) shows the influence of target sequences and guide offsets on the level of indel formation at the OsDEP1 target site in rice protoplasts co-transfected with paired gRNAs and the LbCas12a-RuvC lid deletion nickase variant (SEQ ID NO: 15). Guide offset is defined as the distance between the PAM-distal (3′) ends of the guides of a given gRNA pair.

FIG. 14 (FIG. 14) shows the indel frequencies in rice protoplasts induced by dual nicking with selected Cas12a-nickase variants compared to those induced by a single nickase or WT LbCas12a.

FIG. 15 (FIG. 15) shows a schematic of the transient expression vector used for paired nicking experiments in HEK293 cells.

### DETAILED DESCRIPTION

Based on several iterative rounds of in silico analysis, rational protein design and semi-random saturation mutagenesis approaches, and subsequent functional testing, the inventors have identified several variants of Cas12a, including Lachnospiraceae Cas12a (LbCas12a) that show efficient nicking both in vitro and in vivo and performance of the different variant candidates could be tested using several activity assays in different organisms, including E. coli, plant and yeast and mammalian cell culture systems.

For Cas12a, structural and mechanistic insights are meanwhile available (e.g., Stella et al., Cell, 2018), which studies showed that Cas12a comprises a so-called “lid” protein segment that contains the catalytic E1006 (FnCas12a, SEQ ID NO: 3; corresponds to E925 of LbCas12a, SEQ ID NO: 1) and other residues in the loop that closes the catalytic pocket in the apo structure. During the hybridization of the crRNA guide region and the target DNA strand in Cas12a, certain key motifs such as the finger, helix-loop-helix (HLH), and REC linker from the REC lobe as well as the lid motif in the RuvC domain work concertedly to conformationally activate the DNase activity of Cas12a (Stella et al., 2018; Zhang et al, 2021).

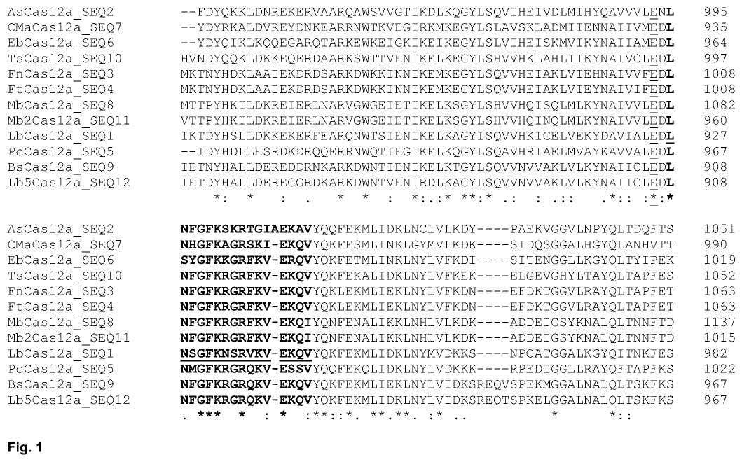

So far, the conformationally flexible portion of the lid domain following the catalytically active residue E925 (LbCas12a; SEQ ID NO: 1) as such highly conserved within all Cas12a orthologs was not yet studied in detail for generating effective Cas12a-based nickases. Therefore, this motif, called the “core lid domain” herein (cf. SEQ ID NO: 13 for the overall consensus sequence) was specifically analyzed as target structure for rational protein design to establish highly functional Cas12a-nickases having an intact catalytically active site, but regulating and fine-tuning nicking activity of only one strand by modifying the lid flexibility. The core lid domain of LbCas12a as reference sequence (cf. SEQ ID NO: 1 and FIG. 1) comprises the core lid domain as defined herein starting with position L927 and ending at position V924. The homologous positions in conserved Cas12a homologs/orthologs known to the skilled person and disclosed herein (e.g., SEQ ID NOs: 1 to 12) can be determined by the skilled person based on the information provided herein.

SEQ ID NO: 13, as detailed in Example 2 below, was identified as a core lid domain and thus a new sub-motif within Cas12a. This core lid domain corresponds to 927 to 942 according to SEQ ID NO:1 (LbCas12a) as reference sequence and it was shown to represent a suitable consensus sequence or motif to characterize and identify Cas12 variants. Therefore, the skilled person can easily identify a Cas12a protein having a core lid domain based in the disclosure presented herein. Based on the in silico analyses detailed in Example 2, the X positions in SEQ ID NO: 13 may correspond to the following sequences in a Cas12a wild-type enzyme in the various aspects and embodiments disclosed herein. Xaa at position 2 of SEQ ID NO: 13 can be a N or S or an amino acid having a similar polarity, the Xaa at position 3 of SEQ ID NO: 13 can be F, H, or Y or an amino acid having a similar polarity, the Xaa at position 7 of SEQ ID NO: 13 can be S, A, K, R, N, or an amino acid having a similar polarity, the Xaa at position 8 of SEQ ID NO: 13 can be K or G, or an amino acid having a similar polarity, the Xaa at position 10 of SEQ ID NO: 13 can be T, S, F, V, Q, or an amino acid having a similar polarity, the Xaa at position 11 of SEQ ID NO: 13 can be G or K, or an amino acid having a similar polarity, the Xaa at position 12 of SEQ ID NO: 13 can be I or V, or an amino acid having a similar polarity, the Xaa at position 13 of SEQ ID NO: 13 can be present or absent, if present, it can be A, or an amino acid having a similar polarity, the Xaa at position 15 of SEQ ID NO: 13 can be K, R, S, or an amino acid having a similar polarity, the Xaa at position 16 of SEQ ID NO: 13 can be A, G, S, or an amino acid having a similar polarity, and the Xaa at position 17 of SEQ ID NO: 13 can be V or I, or an amino acid having a similar polarity.

All wild-type Cas12a enzymes provided so far disclosed in the prior art as suitable for genome editing can qualify as sources for a Cas12a nickase as disclosed herein. As orthologs, for example, closely related FnCas12a, ErCas12a sequences might qualify—without having these included in the independent claims.

Other species sources are: Cas12a variants or any Cas12 ortholog selected from the group consisting of Francisella tularensis, Prevotella albensis, Lachnospiraceae bacterium, Butyrivibrio proteoclasticus, Peregrinibacteria bacterium, Parcubacteria bacterium, Smithella sp., Acidaminococcus sp., Candidatus Methanoplasma termitum, Eubacterium eligens, Eubacterium rectale, Moraxella bovoculi, Leptospira inadai, Porphyromonas crevioricanis, Prevotella disiens and Porphyromonas macacae, Succinivibrio dextrinosolvens, Prevotella disiens, Flavobacterium sp., Flavobacterium branchiophilum, Helcococcus kunzii, Eubacterium sp., Microgenomates (Roizmanbacteria) bacterium, Prevotella brevis, Moraxella caprae, Bacteroidetes oral, Porphyromonas cansulci, Synergistes jonesii, Prevotella bryantii, Anaerovibrio sp., Butyrivibrio fibrisolvens, Candidatus Methanomethylophilus, Butyrivibrio sp., Oribacterium sp., Pseudobutyrivibrio ruminis and Proteocatella sphenisci., Acidibacillus spp., including Acidibacillus sulfuroxidans, Deltaproteobacteria spp, Planctomycetes spp.

In a first aspect according to the present invention there is provided an engineered Cas12a enzyme having nickase activity (nCas12a), or a catalytically active fragment thereof, wherein the engineered Cas12a enzyme may comprise at least one mutation in its core lid domain, wherein the mutation in the core lid domain is selected from: (i) at least three point mutations of three consecutive positions within the core lid domain; or (ii) a deletion of at least two consecutive positions within the core lid domain; or (iii) a combination of at least one first point mutation at at least one position within the core lid domain, including two or more point mutations at consecutives positions, and (iiia) at least one deletion of at least one position, including two or more deletions at consecutive positions, within the core lid domain, and/or (iiib) at least one, preferably at least two, at least three, or at least four further point mutation(s), including two or more point mutations at consecutives positions, at a different position in comparison to the first point mutation within the core lid domain, wherein the position(s) of the further point mutation(s) is/are not in consecutive order with the position(s) of the at least one first point mutation; (iv) one point mutation at a position within the core lid domain; wherein the at least one mutation in the core lid domain confers broad spectrum nickase activity, wherein the core lid domain reference sequence comprises a sequence as defined in SEQ ID NO: 13, optionally a complex additionally comprising at least one compatible guide RNA, or a sequence encoding the same, forming a complex with the cognate engineered Cas12a enzyme having nickase activity, or the catalytically active fragment thereof.

In one embodiment, the at least one mutation in the core lid domain is within positions 5 to 15 with reference to SEQ ID NO: 13.

X or Xaa positions as defined in SEQ ID NO: 13 may be present in similar polarity in another wild-type Cas12a ortholog or homolog. A “similar polarity” as used herein in this context means a polarity according to a standard polarity (that is, the distribution of electric charge) of the side chain of an amino acid, wherein a similar polarity implies that an amino acid residue at a given position may be exchanged against an amino acid within the same polarity group, wherein the polarity groups are selected from: Group I comprising nonpolar amino acids selected from glycine, alanine, valine, leucine, isoleucine, proline, phenylalanine, methionine, and tryptophan; Group II comprising polar, uncharged amino acids, being selected from amino acids serine, cysteine, threonine, tyrosine, asparagine, and glutamine; Group III comprising acidic amino acids selected from aspartic acid and glutamic acid; Group IV comprising basic amino acids selected from arginine, histidine, and lysine.

In one embodiment according to the various aspects as disclosed herein, 1, 2, 3, 4, 5, 6, 7 or all 8 positions 6 to 13 with reference to SEQ ID NO: 13 may be deleted or have a point mutation or a combination thereof.

In one embodiment according to the various aspects as disclosed herein, 1, 2, 3, 4, 5, 6, 7, 8, 9, 10 or all 11 positions 5 to 15 with reference to SEQ ID NO: 13 may be deleted, or they may have a point mutation or a combination thereof.

In one embodiment according to the various aspects as disclosed herein, 1, 2, 3, 4, 5, 6, 7, 8, 9, 10, 11, 12, 13, 14, 15, 16 or all 17 positions of the core lid domain with reference to SEQ ID NO: 13 are deleted or have a point mutation or a combination thereof.

In certain embodiments, the at least one point mutation in the core lid domain according to the present invention may comprise or consist of, at least three point mutations of three positions within the core lid domain, preferably wherein the mutation comprises or consists of (a) a first point mutation at a first position or a first stretch of at least two point mutations at consecutive positions, (b) a second point mutation at a second position or a second stretch of at least two point mutations at consecutive positions, (c) a third point mutation at a third position or a third stretch of at least two point mutations at consecutive positions, and optionally (d) at least one further point mutation at least one further position or at least one further stretch of at least two point mutations at consecutive positions, wherein the first position or first stretch of positions, the second position or second stretch of positions, the third position or third stretch of positions, and optionally the at least one further position or at least one further stretch of positions are not in consecutive order to each other.

In one embodiment according to the various aspects as disclosed herein, the at least one point mutation in the core lid domain according to the present invention may comprise or consist of one deletion at a first position or at least two deletions of a first stretch of consecutive positions, and a second deletion of a second position, or a second stretch of consecutive deletions, and optionally at least one further deletion of least one further position, or at least one further stretch of consecutive deletions, wherein the position of the second deletion or the second stretch of deletions is not in consecutive order with the first deletion or first stretch of consecutive deletions, and optionally wherein the positions of the at least one further deletion or the at least one further stretch of deletions is not in consecutive order with the first position or the first stretch of consecutive positions and the second position or second stretch of consecutive deletions.

In certain embodiments, the at least one point mutation in the core lid domain may comprise or consist of (a) one deletion of one position, two deletions, three deletions, four deletions, five deletions, six deletions, seven deletions, eight deletions, or nine deletions, or in certain embodiments more than nine deletions, of a stretch of consecutive positions, preferably wherein the position or stretch of positions is within positions 5 to 15 with reference to SEQ ID NO: 13, (optionally) in combination with 1, 2, 3, 4, 5, 6, 7, 8, 9, 10, 11, 12, 13, 14, 15, or 16 point mutations, wherein some or all positions of the point mutations may be in consecutive order and may optionally be in consecutive order with the position or stretch of positions of the deletion(s); or (b) a first deletion of a first position or, a first stretch of two, three, four, or five, consecutive deletions of a first stretch of positions, preferably wherein the first position or first stretch of positions is within positions 5 to 15 with reference to SEQ ID NO: 13, and a second deletion of a second position, preferably at least one second stretch of (in total) two, three, four, or five, consecutive deletions of at least one second stretch of positions, preferably wherein the second position or the at least one second stretch of positions is within positions 5 to 15 with reference to SEQ ID NO: 13, optionally wherein the second deletion or at least one second stretch of consecutive deletions is not in consecutive order with the first deletion or first stretch of consecutive deletions, optionally in combination with 1, 2, 3, 4, 5, 6, 7, 8, 9, 10, 11, 12, 13, 14 or 15 point mutations, wherein some or all positions of the point mutations may be in consecutive order and may optionally be in consecutive order with the position or stretch of positions of the deletion of any of the deletions.

In one embodiment according to the various aspects as disclosed herein, the engineered Cas12a enzyme may be based on a wild-type Cas12a sequence according to any one of SEQ ID NOs: 1 to 12, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the corresponding wild-type sequence as reference sequence, or an ortholog or homolog of a sequence according to any one of SEQ ID NOs: 1 to 12 having at least 95%, 96%, 97%, 98% or at least 99% sequence identity to the corresponding ortholog or homolog sequence as reference sequence.

In another embodiment according to the various aspects as disclosed herein, the at least three point mutations in three consecutive amino acids may be positioned within positions 2 to 16 with reference to SEQ ID NO: 13, and/or wherein the deletion is a deletion of at least two, at least three, at least four, at least five, at least six at least seven, at least eight, at least nine, at least ten, at least eleven, at least twelve, at least thirteen, at least fourteen, at least fifteen, at least sixteen, or at least seventeen consecutive positions within the core lid domain.

In another embodiment according to the various aspects as disclosed herein, the mutation may be a deletion of at least four, at least five, at least six at least seven, or at least all eight positions 6 to 13 with reference to SEQ ID NO: 13, and/or wherein the mutation is at least a mutation of three point mutations of three consecutive positions within positions 6 to 13 with reference to SEQ ID NO: 13.

In another embodiment according to the various aspects as disclosed herein, the engineered Cas12a enzyme or the catalytically active fragment thereof has target strand (TS) nickase activity or non-target strand (NTS) nickase activity, preferably, wherein the engineered Cas12a enzyme or the catalytically active fragment thereof has non-target strand (NTS) nickase activity.

In another embodiment according to the various aspects as disclosed herein, the engineered Cas12a enzyme may comprise or may have an amino acid sequence according to SEQ ID NOs: 14 to 21 or 56, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the corresponding reference sequence, or wherein the engineered Cas12a enzyme at least comprises the core lid domain of any one of SEQ ID NOs: 14 to 21 or 56 starting at position 927, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the corresponding core lid domain.

In another embodiment according to the various aspects as disclosed herein, the Cas12a enzyme having nickase activity may comprise at least one further mutation, wherein the at least one further modification modifies the PAM-specificity and/or the thermotolerance of the engineered Cas12a enzyme.

Most wild type Cas12a proteins have a relatively strict requirement for a PAM sequence of TTTV—with some variation between different Cas12a orthologs.

Suitable PAM variants expanding the PAM constraint have been described for various Cas12a orthologs (see for example WO2018195545, WO2020033774, WO2018022634).

According to the various aspects and embodiments disclosed herein, at least one mutation leading to a PAM variant with amended PAM specificity, preferably to expand the PAM constraint of the respective wild-type Cas12a enzyme, can be combined with the nCas12a enzymes as disclosed herein.

Mutants that modify the PAM specificity and/or thermotolerance include, for example, LbCas12a-RR (G532R/K595R), LbCas12a-RVR (G532R/K538V/Y542R), LbCas12a-RVRR (G532R/K538V/Y542R/K595R), enLbCas12a (D156R/G532R/K538R), ttLbCas12a (D156R), FnCas12a-RR (N607R/N617R), FnCas12a-RVR (N607R/K613V/N617R), FnCas12a-RVRR (N607R/K613V/N617R/K671R), AsCas12a-RR (S542R/N552R), AsCas12a-RVR (S542R/K548V/N552R), AsCas12a-RVRR (S542R/K548V/N552R/K607R), enAsCas12a-HF (E174R/N282A/S542R/K548R), MbCas12a-RR (N576R/N582R), MbCas12a-RVR (N576R/K578V/N582R), MbCas12a-RVRR (N576R/K578V/N582R/K634R), Mb2Cas12a-RVR (Mb2Cas12a N563R/K569V/N573R), Mb2Cas12a-RVRR (Mb2Cas12a N563R/K569V/N573R/K625R), BsCas12a-3Rv (K155R/N512R/K518R), PrCas12a-3Rv (E162R/N519R/K525R), Mb3Cas12a-3Rv (D180R/N581R/K587R) (WO2018195545, WO2020033774, WO201822634).

In some embodiments according to the various aspects as disclosed herein, the at least one mutation in the core lid domain according to the present invention may be present in a Cas12a variant with one of the following amino acid reference sequences: SEQ ID NO: 27, SEQ ID NO: 28, SEQ ID NO: 29, SEQ ID NO: 30, SEQ ID NO: 31, SEQ ID NO: 32 or SEQ ID NO: 33.

In one embodiment, at least one mutation, preferably exactly one, mutation introduced into the core lid domain motif may insert a Cys residue instead of the wild-type amino acid, wherein the at least one inserted Cys residue, preferably the exactly one inserted Cys residue, may be introduced in combination with one or more other point mutation(s) and/or deletion(s) according to the present invention. Without wishing to be bound by theory, it is assumed that the introduction of an additional cysteine residue can favourably change the dynamic lid domain reassortment upon binding of the DNA target site so that the nickase activity is promoted.

In certain embodiments, the nCas12a or active fragment thereof, does not comprise a point mutation at position 6 (with reference to SEQ ID NO: 13) resulting in a glycine residue in combination with a point mutation at position 7 (with reference to SEQ ID NO: 13) resulting in a glycine residue, without comprising at least one further point mutation and or deletion within the within the core lid domain (SEQ ID NO: 13).

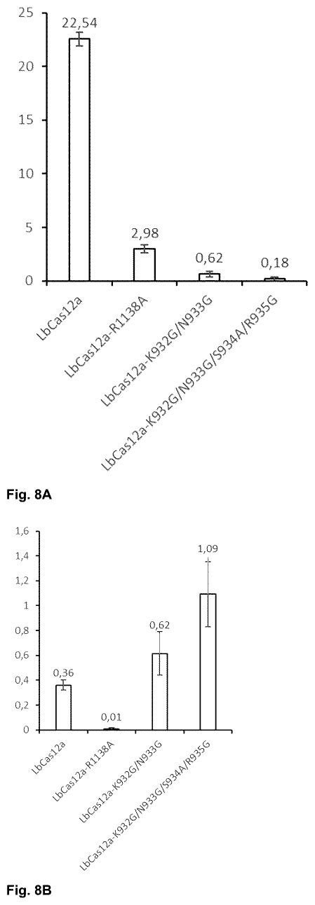

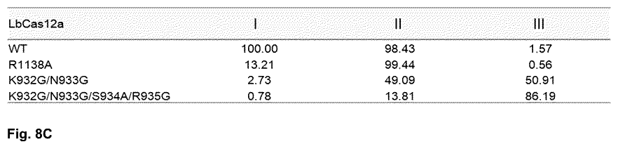

In certain embodiments, a Cas12a enzyme as disclosed herein having nickase activity and comprising a flexible lid domain may also be selected from an ortholog of Cas12a having—in its natural environment—the same overall functionality as a Class 2 type V CRISPR nuclease and having the same overall fold and mechanistic action as Cas12a. Particularly, such an ortholog will have a lid domain dynamically opening and closing upon substrate binding exactly in a way as Cas12a (Stella et al., 2017) so that also the lid domains of these Cas12a ortholog nickase effectors can be modified and used as disclosed herein. As shown in Zhang et al. for the Cas12a ortholog Cas12i (2020; cf. Extended Suppl. Data FIG. 8), a lid domain seems to be conserved in Cas12a orthologs of class 2 type V CRISPR effectors so that the findings herein can be extended to a sub-motif within the core lid domain as defined herein.

In one embodiment, a nCas12a ortholog enzyme may include Cas12e (also referred to as CasX), including DpbCas12e and PlmCas12e (Selkova et al. RNA Biol. (2020); 17(10):1472-1479; doi: 10.1080/15476286.2020.1777378).

In another embodiment, a nCas12a ortholog enzyme may include Cas12f variants, including Cas12f1 (Cas14a and type V-U3), including AsCas12f1 and Un1Cas12f1, Cas12f2 (Cas14b) and Cas12f3 (Cas14c, type V-U2 and U4) (Kim et al. Nat Biotechnol. (2022); 40(1):94-102; doi: 10.1038/s41587-021-01009-z; Karvalis et al. Nucleic Acids Res. (2020); 48(9):5016-5023. doi: 10.1093/nar/gkaa208).

In a second aspect, there is provided a nucleic acid sequence or nucleic acid molecule (used interchangeably herein in the context of a Cas12a enzyme or a catalytically active fragment or variant thereof) encoding the Cas12a enzyme or the catalytically active fragment thereof according to the first aspect of the invention, optionally, wherein the nucleic acid sequence is a codon-optimized sequence and/or comprises a nucleic acid sequence encoding at least one guide RNA.

In some embodiments, the nucleic acid sequence is codon-optimized for a fungal cell, including a yeast cell, a prokaryotic cell or an archea cell, in particular for a fungal cell, a prokaryotic cell or an archea cell disclosed herein. In one embodiment, the nucleic acid molecules comprises or consists of a fungal- or prokaryotic-optimized sequence according to SEQ ID NOs: 80 to 87, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99%. SEQ ID NOs: 80 to 87 are sequences encoding the LbCas12a-RuvC lid deletion, codon-optimized for Bacillus subtilis, Rhodococcus spp., Yarrowia lipolytica, Escherichia coli K12, Saccharomyces cerevisiae, Rhodobacter sphaeroides, Corynebacterium glutamicum and Pseudozyma tsukubaensis, respectively. The sequences have been adapted by adaptation according to the fraction of the codon usage table of the selected organism and removal of repeats of the same codons are removed to avoid stalling of translation.

In some embodiments, the nucleic acid sequence is codon-optimized for a plant cell as disclose, in particular for a plant cell disclosed herein. In one embodiment, the nucleic acid molecules comprises or consists of a plant-optimized sequence according to SEQ ID NOs: 88 to 93, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99%. SEQ ID NOs: 88 to 93 are sequences encoding the LbCas12a-RuvC lid deletion, codon-optimized for Glycine max, Zea mays, Brassica napus, Gossypium spp, Oryza sativa and Triticum aestivum, respectively. The sequences have been codon-optimized by using GeneOptimizer, a BASF proprietary adaptation method according to the fraction of the codon usage table of the selected organism.

In some embodiments, the nucleic acid sequence is codon-optimized for an animal cell, including human cell, in particular for an animal cell, including human cell, disclosed herein. In one embodiment, the nucleic acid molecules comprises or consists of an animal-optimized sequence according to SEQ ID NOs: 94 to 99, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99%. SEQ ID NOs: 94 to 99 are sequences encoding the LbCas12a-RuvC lid deletion, codon-optimized for Homo sapiens, Rattus norvegicus, Bos taurus, Mus musculus, Sus scrofa and Gallus gallus, respectively. The sequences have been adapted by using the CLC Genomics Workbench reverse translate tool, based on frequency distribution

The nucleic acid sequence may be operably linked to a promoter sequence and/or a terminator sequence that is suitable for a desired target cell in which the provided nucleic acid sequence might be expressed.

In a third aspect, there is provided an expression construct or vector comprising at least one nucleic acid sequence according to the second aspect.

Expression constructs or vectors suitable for a multitude of different target cells as well as means and methods to design such expression constructs or vectors, including a large variety of suitable markers, are well known to the skilled person.

Non-limiting examples of classes of expression constructs and vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self transmissible or mobilizable. In some embodiments, a viral vector can include, but is not limited, to a retroviral, lentiviral, adenoviral, adeno-associated, or herpes simplex viral vector.

In a fourth aspect, there is provided a cell comprising at least one nucleic acid sequence according to the second aspect, or comprising at least one expression construct or vector according to the third aspect.

In one embodiment, the cell may be a eukaryotic cell or a prokaryotic cell, including a bacterial or an archaea cell.

A cell, particularly for a multicellular organism, as used herein is preferably an isolated and/or cultured cell that can be analyzed and modified.

In one embodiment according to the various aspects as disclosed herein, the cell may be a plant cell, including an algal cell, preferably wherein the cell may be selected from a cell originating from a plant which belongs to the superfamily Viridiplantae, in particular monocotyledonous and dicotyledonous plants including but not limited to fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

Preferred plants may be independently selected from Abelmoschus spp., Allium spp., Apium graveolens, Asparagus officinalis, Avena spp. (e.g. Avena sativa, Avena fatua, Avena byzantina, Avena fatua var. sativa, Avena hybrida), Beta vulgaris, Brassica spp. (e.g. Brassica napus, Brassica rapa ssp. [canola, oilseed rape, turnip rape]), Capsicum spp., Citrullus lanatus, Cucumis spp., Cynara spp., Daucus carota, Glycine spp. (e.g. Glycine max, Soja hispida or Soja max), Gossypium hirsutum, Helianthus spp. (e.g. Helianthus annuus), Hordeum spp. (e.g. Hordeum vulgare), Lactuca sativa, Medicago sativa, Oryza spp. (e.g. Oryza sativa, Oryza latifolia), Pennisetum sp., Saccharum spp., Secale cereale, Solanum spp. (e.g. Solanum tuberosum, Solanum integrifolium or Solanum lycopersicum), Sorghum bicolor, Spinacia spp., Triticum spp. (e.g. Triticum aestivum, Triticum durum, Triticum turgidum, Triticum hybernum, Triticum macha, Triticum sativum, Triticum monococcum or Triticum vulgare), or Zea mays.

Other preferred plants may be selected from Brassica spp. (e.g. Brassica napus, Brassica rapa ssp. [canola, oilseed rape, turnip rape]), Capsicum spp., Glycine spp. (e.g. Glycine max, Soja hispida or Soja max), Gossypium hirsutum, Helianthus spp. (e.g. Helianthus annuus), Oryza spp. (e.g. Oryza sativa, Oryza latifolia), Solanum spp. (e.g. Solanum tuberosum, Solanum integrifolium or Solanum lycopersicum), Triticum spp. (e.g. Triticum aestivum, Triticum durum, Triticum turgidum, Triticum hybernum, Triticum macha, Triticum sativum, Triticum monococcum or Triticum vulgare), or Zea mays.

The term “plant” as used herein encompasses whole plants, ancestors and progeny of the plants and plant parts, including seeds, shoots, stems, leaves, roots (including tubers), flowers, and tissues and organs. The term “plant” also encompasses plant cells, suspension cultures, callus tissue, embryos, meristematic regions, gametophytes, sporophytes, pollen and microspores.

A plant cell, tissue, organ, material, or whole organism as used herein includes an algal cell, tissue, organ, material or whole organism, respectively.

In another embodiment according to the various aspects as disclosed herein, the cell may be an animal cell, including an insect, poultry, fish or crustacea cell, or a mammalian cell, preferably wherein the cell is a mammalian cell; optionally being selected from a cell originating from a non-human primate, bovine, porcine, rodent, including rat or mouse, or human cell.

An animal cell, tissue, organ, or material as used herein includes a human cell, tissue, organ, or material, respectively.

In another embodiment according to the various aspects as disclosed herein, the cell may be a fungal cell, including a yeast cell, preferably wherein the fungal cell, including the yeast cell, is selected from a cell originating from Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Aspergillus spec. such as Aspergillus niger or Myceliophthora thermophila.

In yet another embodiment according to the various aspects as disclosed herein, the cell may be a prokaryotic cell, including Gram-positive, Gram negative and Gram-variable bacterial cells, preferably Gram-negative bacterial cells, or an archaea cell, preferably wherein the prokaryotic cell is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, such as Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp.

In a preferred embodiment according to the various aspects as disclosed herein, the cell may be a eukaryotic cell or a prokaryotic cell, wherein the cell is selected from a cell originating from Rhodococcus rhodochrous, Aerococcus sp., Ashbya gossypii, Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica, wherein the cell is preferrably selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae or Yarrowia lipolytica.

In another embodiment, the cell may be a eukaryotic cell or a prokaryotic cell, wherein the cell is selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica, Phakopsora spec, e.g. Phakopsora pachyrhizi, Zymoseptoria spec, e.g. Zymoseptoria tritici, Septoria, Mycosphaerella, Phythopthora spec., e.g. Phytopthora infestans, Puccinia, Sphaerotheca, Blumeria, Erysiphe, Alternaria, Botrytis, Ustilago, Venturia, Verticillium, Pyricularia, Magnaporthe, Plasmopara, Pythium, Sclerotinia, Colletotrichum, Penicillium, Neurospora, Aspergillus, or Ashbya.

In a fifth aspect, there is provided a complex, or at least one nucleic acid sequence encoding the components of the complex, the complex comprising at least one engineered Cas12a enzyme having nickase activity or a catalytically active fragment according to the first aspect of the present invention, and at least one compatible guide RNA, optionally comprising at least one further polypeptide, covalently and/or non-covalently attached to the at least one engineered Cas12a enzyme having nickase activity or the catalytically active fragment thereof within the complex, wherein the at least one further polypeptide is selected from an organellar localization sequence, including a nuclear localization signal (NLS), a mitochondrion localization signal, or a chloroplast localization signal, and/or wherein the at least one further polypeptide is a cell-penetrating polypeptide, preferably, in case the at least one further polypeptide is covalently attached to the at least one engineered Cas12a enzyme having nickase activity or the catalytically active fragment thereof, wherein the at least one further polypeptide is covalently attached to the N-terminus and/or the C-terminus of the at least one engineered Cas12a enzyme having nickase activity.

In a sixth aspect, there is provided a fusion protein or at least one nucleic acid sequence encoding the same, comprising at least one engineered Cas12a enzyme having nickase activity or the catalytically active fragment thereof according to the first aspect of the present invention, covalently and/or non-covalently attached to at least one further polypeptide domain, the at least one further polypeptide domain having an activity selected from an enzymatic activity, binding activity or targeting activity, and optionally comprising at least one guide RNA compatible with the engineered Cas12a enzyme having nickase activity, wherein the at least one compatible guide RNA covalently and/or non-covalently interacts with the at least one engineered Cas12a enzyme having nickase activity or the catalytically active fragment thereof.

The nCas12a fusion protein of the invention may be a chimeric nCas12a protein functionally linked, preferably fused to a polypeptide sequence comprising at least one heterologous polypeptide that has enzymatic activity that modifies at least one target nucleic acid (e.g., nuclease activity, e.g. exonuclease activity, methyltransferase activity, demethylase activity, DNA repair activity, DNA damage activity, deamination activity, dismutase activity, alkylation activity, depurination activity, oxidation activity, pyrimidine dimer forming activity, helicase activity (e.g. SF1/2, SF3, SF4), integrase activity, telomerase activity, topoisomerase activity, e.g. gyrase activity, transposase activity, transcriptase or reverse transcriptase activity, recombinase activity, polymerase activity, e.g. RNA polymerase activity or DNA polymerase activity e.g. Pol theta activity, ligase activity, photolyase activity or glycosylase activity).

In some cases, a chimeric nCas12a fusion protein may comprise at least one heterologous polypeptide that has enzymatic activity that modifies at least one protein and/or polypeptide (e.g., a histone) associated with at least one target nucleic acid. Examples of enzymatic activity that modifies at least one protein and/or polypeptide associated with at least one target nucleic acid that can be provided by the fusion partner include but are not limited to: methyltransferase activity, such as that provided by a histone methyltransferase (HMT) (e.g., suppressor of variegation 3-9 homolog 1 (SUV39H1 or KMT1A), euchromatic histone lysine methyltransferase 2 (G9A, KMT1C, EHMT2), SUV39H2, ESET/SETDB 1, and the like, SET1A, SET1B, MLL1 to 5, ASH1, SYMD2, NSD1, DOT1 L, Pr-SET7/8, SUV4-20H1, EZH2), demethylase activity such as that provided by a histone demethylase (e.g., Lysine Demethylase 1A (KDM1A also known as LSD1), JHDM2a/b, JMJD2A/JHDM3A, JMJD2B, JMJD2C/GASC1, JMJD2D, JARID1A/RBP2, JARID1B/PLU-1, JARID1C/SMCX, JARID1D/SMCY, UTX, JMJD3, and the like), acetyltransferase activity, such as that provided by a histone acetylase transferase (e.g., catalytic core/fragment of the human acetyltransferase p300, GCN5, PCAF, CBP, TAF1, TIP60/PLIP, MOZ/MYST3, MORF/MYST4, HB01/MYST2, HMOF/MYST1, SRC1, ACTR, P160, CLOCK and the like), deacetylase activity, such as that provided by a histone deacetylase (e.g., HDAC1, HDAC2, HDAC3, HDAC8, HDAC4, HDAC5, HDAC7, HDAC9, SIRT1, SIRT2, HDAC11, and the like), kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, and demyristoylation activity.

In some embodiments, the fusion partner may have enzymatic activity that modifies at least one target nucleic acid. Examples of enzymatic activity include but are not limited to: nuclease activity, such as that provided by a restriction enzyme (e.g., Fokl nuclease, C10051 nuclease, homing endonucleases), DNA repair activity, DNA damage activity, deamination activity such as that provided by a deaminase (e.g., a cytosine deaminase such as rat APO-BEC1 or adenine deaminase), dismutase activity, alkylation activity, depurination activity, oxidation activity, pyrimidine dimer forming activity, integrase activity such as that provided by an integrase and/or resolvase (e.g., Gin integrase such as the hyperac-tive mutant of the Gin integrase, GinH106Y; human immunodeficiency virus type 1 integrase (IN); Tn3 resolvase; and the like), transposase activity, recombinase activity, such as that provided by a recombinase (e.g., catalytic domain of Gin recombinase, Cre recombinase, Hin recombinase, Tre recombinase, FLP recombinase, RecA, RadA, Rad51), polymerase activity (e.g. RNA polymerase activity, DNA polymerase activity), ligase activity, helicase activity, photolyase activity, or glycosylase activity.

In some cases, an nCas12a fusion protein may comprise at least one detectable label. Suitable detectable labels and/or moieties that can provide a detectable signal can include, but are not limited to, an enzyme, a radioisotope, a member of a specific binding pair, a fluorophore, a fluorescent protein, a quantum dot, and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPI, pocilloporin, Renilla GFP, Monster GFP, paGFP, Kaede protein and kindling protein, Phycobiliproteins and Phycobiliprotein conjugates including BPhycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. 2005), and the like.

Suitable enzymes that may function as a detectable label include, but are not limited to, horse radish peroxidase (HRP), alkaline phosphatase (AP), beta-galactosidase (GAL), glucose-6-phosphate dehydro-genase, beta-Nacetylglucosarninidase, f3-glucuronidase, invertase, Xanthine Oxidase, firefly luciferase, glucose oxidase (GO), and the like.

Further suitable fusion partners include but are not limited to proteins (or fragments thereof) that are boundary elements (e.g., CTCF), proteins and fragments thereof that provide periphery recruitment (e.g., Lamin A, Lamin B, etc.), protein docking elements (e.g., FKBP/FRB, Pill/Abyl, etc.).

In certain embodiment, the at least one nucleic acid sequence encoding the fusion protein is codon optimized.

In a seventh aspect of the present invention there is provided an adenine or a cytidine base editor, or a base editor complex, or at least one nucleic acid sequence encoding the same, the base editor or base editor complex comprising at least one catalytically active portion of at least one engineered Cas12a enzyme having nickase activity according to the first aspect of the present invention.

A “base editor” as used herein refers to a protein or a catalytically active fragment thereof, which can—together with a compatible guide RNA—induce a targeted base modification, i.e., the conversion of at least one base into at least one different base, thereby resulting in one or more point mutations. A “base editor complex” refers to a system that comprises at least two non-covalently attached components, which can function as a base editor together. Base editors are frequently used in form of a base editor complex. Base editors, for example CBEs (cytosine base editors mediating C to T conversion) and ABEs (adenine base editors mediating A to G conversion), are powerful tools to introduce direct mutations without the need for DSB induction (Komor et al., Nature, 2016, 533(7603), 420-424; Gaudelli et al., Nature, 2017, 551, 464-471). Base editors or base editor complexes are composed of at least one DNA targeting module, such as a Cas protein or functional fragment thereof together with at least one a suitable guide RNA, and at least one catalytic deaminase module, which deaminates cytidine and/or adenine. All four transition mutations of DNA (C•G to T•A to A•T to G•C) are possible—depending on the choice of deaminase, and possible combination thereof. Both CBEs and ABEs have been optimized and applied in various cellular systems, including mammalian cells and plants (Fan et al., Communications Biology (2021), 4(1):882, doi: 10.1038/s42003-021-02406-5; Zong et al., Nature Biotechnology, vol. 25, no. 5, 2017, 438-440; Yan et al., Molecular Plant, vol. 11, 4, 2018, 631-634; Hua et al., Molecular Plant, vol. 11, 4, 2018, 627-630).

The terms “cytosine base editor (complex)” and “cytidine base editor (complex)” are used interchangeably herein. Likewise, “cytosine deaminase” and “cytidine deaminase” are used interchangeably herein.

The terms “adenosine base editor (complex)” and “adenine base editor (complex)” are used interchangeably herein. Likewise, “adenosine deaminase” and “adenine deaminase” are used interchangeably herein.

In one embodiments of the present invention the at least one deaminase module is fused covalently to the nCas12a or catalytically active fragment thereof, optionally as a complex further comprising at least one compatible guide RNA, wherein the deaminase module may be fused C-terminally or N-terminally or internally to the nCas12a or catalytically active fragment thereof, wherein each module may be separated from other modules by a suitable linker or spacer region as these are known to the skilled person. Covalent fusion of the different modules of the base editor is usually achieved by cloning a nucleic acid sequence encoding the desired modules and (optionally) linker sequences.

In another embodiment, the at least one deaminase module may be non-covalently attached to the nCas12a or catalytically active fragment thereof, optionally as a complex further comprising at least one compatible guide RNA. Methods of non-covalent attachment, such as protein binding domains and the like, are well known to the skilled person.

In certain embodiments, the at least one deaminase module may be covalently or non-covalently attached to at least one compatible guide RNA that is able to form a complex with at least one nCas12a or catalytically active fragment thereof.

In certain embodiments, at least one further polypeptide may be covalently and/or non-covalently attached to the at least one base editor or base editor complex, wherein the at least one further polypeptide comprises a glycosylase inhibitor activity, such as a uracil glycosylase inhibitor (UGI), a glycosylase activity, such as a uracil DNA glycosylase (UDG), including a uracil-n-glycosylase (UNG), an organellar localization sequence, including a nuclear localization signal (NLS), a mitochondrion localization signal, or a chloroplast localization signal, or a cell-penetrating polypeptide, or any combination thereof, including the combination of more than one polypeptide sequences of the same type, including the combination of more than one identical polypeptide sequences, wherein a further polypeptide or further polypeptides that is/are attached covalently, is/are attached N-terminally, c-terminally or internally to the base editor or base editor complex, wherein each functional module and/or domain may be separated from one or more other functional module(s) and/or domains(s) by at least one linker region. In embodiments relating to a base editor complex, all protein components of the base editor complex may each be (covalently and/or non-covalently) attached to the same type of, or identical, organellar localization sequences.

A variety of adenine and cytosine deaminases are known to the skilled person (e.g. Fan et al., Communications Biology (2021), 4(1):882, doi: 10.1038/s42003-021-02406-5; Jeong et al., Molecular Therapy (2020), 28(9):1938-1952, doi: 10.1016/j.ymthe.2020.07.021; Yan et al., Molecular Plant (2021), 14(5):722-731, doi: 10.1016/j.molp.2021.02.007). Any adenine deaminase and/or cytosine deaminase, including variants of known deaminases may be used in a base editor or base editor complex using any nCas12a of the present invention.

In one embodiment the at least one deaminase module comprises at least one adenine deaminase or domain or thereof. In another embodiment the at least one deaminase module comprises at least one cytosine deaminase or domain thereof. In yet another embodiment, the at least one deaminase module comprises at least one adenine deaminase or domain or thereof and at least one cytosine deaminase or domain thereof.

In some embodiments, an adenine deaminase may be a tRNA-specific adenosine deaminase, such as TadA (Gaudelli et al., Nature (2017), 551(7681):464-471, doi: 10.1038/nature24644), or an adenosine deaminase 1 (ADA1), ADA2; an adenosine deaminase acting on RNA 1 (ADAR1), ADAR2, ADAR3 (e.g., Savva et al., Genome Biol. 2012 Dec. 28; 13(12):252); or an adenosine deaminase acting on tRNA 1 (ADAT1), ADAT2, ADAT3, or variant thereof.

In some embodiments, a TadA may be from E. coli. In some embodiments, the TadA may be modified and/or truncated. In certain embodiments, a TadA does not comprise an N-terminal methionine. TadA deaminases that may for be used as part of a base editor or base editor complex according to the present invention may for example be a TadA8, TadA8e, TadA8 s, TadA7.9 TadA7.10, TadA7.10d, TadA8.17, TadA8.20, TadA9, or a variant thereof.

In some embodiments, a cytosine deaminase may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, an activation induced deaminase (AID), such as hAID or AICDA, rAPOBEC1, an PpAPOBEC1, an AmAPOBEC1, an SsAPOBEC3B, an RrA3F, a FERNY, a cytosine deaminase, such as CDA1, CDA2, pmCDA1, or atCDA1, or a cytosine deaminase acting on rRNA (CDAT), or a variant thereof.

In one embodiment, the at least one nucleic acid sequence encoding the base editor or base editor complex may be codon-optimized and may further comprise a nucleic acid sequence encoding at least one compatible guide RNA.

In an eighth aspect, there is provided a prime editor or a prime editor complex, or at least one nucleic acid sequence encoding the same, the prime editor or prime editor complex comprising at least one catalytically active portion of at least one engineered Cas12a enzyme having nickase activity according to the first aspect of the present invention.

Prime editing enables the introduction of indels and all 12 base-to-base conversions without the need to introduce a DSB. For prime editing, a so-called prime editing guide RNA (pegRNA) is used. The pegRNA usually comprises a primer binding site (PBS) and reverse transcriptase (RT) template sequence that will be introduced to the targeted gene. The PBS region is complementary to the non-target strand and will create a primer for RT that is linked to the Cas protein. Subsequently, the sequence of the RT template sequence is copied from the pegRNA into target DNA sequence. Three generations of prime editors have been used in different target cells: PE1, PE2 and PE3. PE1 is based on the Moloney murine leukemia virus reverse transcriptase (M-MLV RT). PE2 (called pPE2 in plants) is based on the M-MLV RT D200N/L603W/T330P/T306K/W313F variant. PE3 (called pPE3 in plants) uses an additional guide RNA specifically targeting the edited sequence (Marzec et al 2020; Xu et al. 2020; Lin et al. 2020). It has also been shown, that the M-MLV RT can also be exchanged by different RTs, such as Cauliflower Mosaic Virus (CaMV) RT, or retron-derived RT (Lin et al. 2020).

In one embodiment according to the various aspects disclosed herein, at least one reverse transcriptase may be fused to at least one nCas12a to form a prime editor, optionally as a complex further comprising at least one compatible pegRNA, wherein the at least one reverse transcriptase is N-terminally, C-terminally or internally fused to the nCas12a, wherein the at least one reverse transcriptase may be connected to the nCas12a via a linker region.

In another embodiment, at least one reverse transcriptase may be non-covalently attached to at least one nCas12a variant of the present invention, optionally as a complex further comprising at least one compatible pegRNA. Methods of non-covalent attachment, such as protein binding domains and the like, are well known to the skilled person.

In certain embodiments, the at least one reverse transcriptase may be covalently or non-covalently attached to at least one compatible pegRNA that is able to form a complex with at least one nCas12a or catalytically active fragment thereof.

In another embodiment, at least one nCas12a or an active fragment thereof and/or at least one reverse transcriptase may comprise at least one further polypeptide, covalently and/or non-covalently attached to the at least one nCas12a or active fragment thereof and/or the at least one reverse transcriptase, wherein the at least one further polypeptide is selected from an organellar localization sequence, including a nuclear localization signal (NLS), a mitochondrion localization signal, or a chloroplast localization signal, and/or wherein the at least one further polypeptide is a cell-penetrating polypeptide, preferably, in case the at least one further polypeptide is covalently attached to the at least one nCas12a or active fragment thereof and/or the at least one reverse transcriptase, wherein the at least one further polypeptide is covalently attached to the N-termially and/or C-terminally and/or internally to the at least nCas12a or active fragment thereof and/or at least on reverse transcriptase. In embodiments relating to a prime editor complex, all protein components of the prime editor complex may each be (covalently and/or non-covalently) attached to the same type of, or identical, organellar localization sequences.

In certain embodiments, the at least one nucleic acid sequence encoding the prime editor or prime editor complex may be codon-optimized and may further comprise a sequence encoding at least one compatible pegRNA and, moreover, may comprise a sequence encoding an additional guide RNA targeting the edited sequence.

In a ninth aspect, there is be provided a kit comprising (i) an engineered Cas12a enzyme having nickase activity (nCas12a), or a catalytically active fragment thereof as defined in the first aspect of the present invention, or an expression construct or vector as defined in the third aspect of the present invention, or a complex as defined in the fifth aspect of the present invention, or at least one sequence encoding the same, or a fusion protein as defined in the sixth aspect of the present invention, or at least one sequence encoding the same, or an adenine or a cytidine base editor, or a base editor complex, or at least one nucleic acid sequence encoding the same as defined in the seventh aspect of the present invention, or prime editor or a prime editor complex, or at least one nucleic acid sequence encoding the same as defined in the eighth aspect of the present invention; (ii) at least one compatible guide RNA, or a set of compatible guide RNAs, each guide RNA being complementary to target sequences of interest; and (iii) a set of reagents; (iv) optionally comprising particles, vesicles, or at least one viral vector, or Agrobacterium vector for assisting delivery, wherein said particles comprise a lipid, including lipid nanoparticles, a sugar, a metal or a polypeptide, or a combination thereof, or wherein said vesicles comprise exosomes or liposomes.

In a tenth aspect, there is provided a method for modifying the genomic locus of interest of at least one cell or construct at or near at least one target site, the method comprising: (a) providing at least one cell or construct comprising the genomic locus to be modified; (b) providing and/or introducing (i) at least one engineered Cas12a enzyme having nickase activity (nCas12a), or a catalytically active fragment thereof, or at least one nucleic acid sequence encoding the same, as defined in the first aspect of the present invention; or (ii) at least one expression construct or vector as defined in the third aspect of the present invention; or (iii) at least one complex or at least one nucleic acid sequence encoding the same as defined in the fifth aspect of the present invention, or at least one fusion protein or at least one nucleic acid sequence encoding the same as defined in the sixth aspect of the present invention; or (iv) at least one adenine or a cytidine base editor, or at least one base editor complex, or at least one nucleic acid sequence encoding the same as defined in the seventh aspect of the present invention; or (v) at least one prime editor or at least one prime editor complex, or at least one nucleic acid sequence encoding the same as defined in the eighth aspect of the present invention; to/into the at least one cell or construct; (c) providing and/or introducing at least one compatible guide RNA, or a sequence encoding the same, as defined in the first aspect of the present invention; (d) allowing complex formation of the at least one engineered Cas12a enzyme having nickase activity, or the catalytically active fragment thereof of (a) and the at least compatible guide RNA as defined in the first aspect of the present invention (b) and thus allowing the insertion of at least one nick at the genomic locus of interest of the at least one cell or construct at or near at least one target site; (e) optionally: providing at least one donor repair template, or at least one the nucleic acid sequence encoding the same; and (f) obtaining at least one edited cell or construct comprising a modification of a genomic locus of interest at or near a target site; wherein the method excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes, optionally, where the method comprises the following step: (g) regenerating at least one population of edited cells, tissues, organs, materials or whole organisms from the at least one edited cell or construct.

In certain embodiments, the at least one nCas12a or active fragment thereof according to the first or fifth aspect, or the at least one fusion protein according to the sixth aspect or the at least one base editor or base editor complex according to the seventh aspect, or the at least one prime editor or prime editor complex according to the eighth aspect may be provided/introduced to/into the at least one cell or construct as a complex with at least one compatible guide RNA, or as at least one nucleic acid encoding said complex, wherein the at least one nucleic acid encoding said complex may be part of at least one vector, wherein the at least one compatible guide RNA may be a pegRNA.

In certain embodiments, the at least one nCas12a or active fragment thereof according to the first or fifth aspect, or the at least one fusion protein according to the sixth aspect or the at least one base editor or base editor complex according to the seventh aspect, or the at least one prime editor or prime editor complex according to the eighth aspect are provided/introduced to/into the at least one cell or construct as a nucleic acid encoding the same, wherein said nucleic acid may further encode at least one compatible guide RNA according to the first aspect or fifth aspect and wherein the at least one nucleic acid may be part of as least one vector, wherein the at least one compatible guide RNA may be a pegRNA. Alternatively, the nCas12a, fusion protein, base editor or base editor complex, or prime editor or prime editor complex, and the at least one compatible guide RNA may be encoded by two separate nucleic acids, which may be provided/introduced to/into the cell or construct simultaneously or separately.

Step (c) of providing and/or introducing at least compatible guide RNA, or a sequence encoding the same may already be fulfilled by providing and/or introducing at least one complex or nucleic acid encoding the same in step (b) that contains at least one compatible guide RNA (including a pegRNA) or nucleic acid encoding the same, so that the provision and/or introduction of at least one (additional) compatible guide RNA or a sequence encoding the same may not be necessary.

In yet another embodiment relating to the provision/introduction of a prime editor or prime editor complex, the at least one compatible guide RNA is a pegRNA, comprising a PBS region and/or a RT template region, optionally wherein there is further provided and/or introduced an additional guide RNA targeting the edited strand, wherein the at least one prime editor or prime editor complex, the at least one pegRNA and optionally the at least one additional guide RNA may be provided and/or introduced by as at least one nucleic acid encoding the same, wherein the at least one nucleic acid may be part of at least one vector.

In certain embodiments, the method of the tenth aspect of the present invention does not lead to the introduction of a DSB in the genomic locus of interest, which is achieved by the outstanding specific nickase activity (and the lack of the wild-type DSB activity) of the nCas12a variants as disclosed herein.

In one embodiment, the method is performed in vitro or in vivo and/or ex vivo.

In certain embodiments, the method does not comprise treatment of the human or animal body by therapy.

In another embodiment, the cell or construct originates from a prokaryotic cell, including a bacterial or an archaea cell, or a eukaryotic cell.

In certain embodiments, the cell may be a plant cell, including an algal cell, preferably wherein the cell is selected from a cell originating from a plant which belongs to the superfamily Viridiplantae, in particular monocotyledonous and dicotyledonous plants including but not limited to fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

Preferred plants may be selected from Abelmoschus spp., Allium spp., Apium graveolens, Asparagus officinalis, Avena spp. (e.g. Avena sativa, Avena fatua, Avena byzantina, Avena fatua var. sativa, Avena hybrida), Beta vulgaris, Brassica spp. (e.g. Brassica napus, Brassica rapa ssp. [canola, oilseed rape, turnip rape]), Capsicum spp., Citrullus lanatus, Cucumis spp., Cynara spp., Daucus carota, Glycine spp. (e.g. Glycine max, Soja hispida or Soja max), Gossypium hirsutum, Helianthus spp. (e.g. Helianthus annuus), Hordeum spp. (e.g. Hordeum vulgare), Lactuca sativa, Medicago sativa, Oryza spp. (e.g. Oryza sativa, Oryza latifolia), Pennisetum sp., Saccharum spp., Secale cereale, Solanum spp. (e.g. Solanum tuberosum, Solanum integrifolium or Solanum lycopersicum), Sorghum bicolor, Spinacia spp., Triticum spp. (e.g. Triticum aestivum, Triticum durum, Triticum turgidum, Triticum hybernum, Triticum macha, Triticum sativum, Triticum monococcum or Triticum vulgare), or Zea mays.

Other preferred plants may be selected from Brassica spp. (e.g. Brassica napus, Brassica rapa ssp. [canola, oilseed rape, turnip rape]), Capsicum spp., Glycine spp. (e.g. Glycine max, Soja hispida or Soja max), Gossypium hirsutum, Helianthus spp. (e.g. Helianthus annuus), Oryza spp. (e.g. Oryza sativa, Oryza latifolia), Solanum spp. (e.g. Solanum tuberosum, Solanum integrifolium or Solanum lycopersicum), Triticum spp. (e.g. Triticum aestivum, Triticum durum, Triticum turgidum, Triticum hybernum, Triticum macha, Triticum sativum, Triticum monococcum or Triticum vulgare), or Zea mays.

In other embodiments, the cell may be a fungal cell, including a yeast cell, preferably wherein the fungal cell, including the yeast cell, is selected from a cell originating from to Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ashbya spec, such as Ashbya gossypii, Ogataea spec such as Ogataea minuta, Aspergillus spec. such as Aspergillus niger or Myceliophthora thermophila.

In certain embodiments, the cell is a eukaryotic cell or a prokaryotic cell, wherein the cell may be selected from a cell originating from Rhodococcus rhodochrous, Aerococcus sp., Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica, wherein the cell is prefererably selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae or Yarrowia lipolytica.

In certain embodiments, the cell is a eukaryotic cell or a prokaryotic cell, wherein the cell may be selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica, Phakopsora spec, e.g. Phakopsora pachyrhizi, Zymoseptoria spec, e.g. Zymoseptoria tritici, Septoria, Mycosphaerella, Phythopthora spec., e.g. Phytopthora i infestans, Puccinia, Sphaerotheca, Blumeria, Erysiphe, Alternaria, Botrytis, Ustilago, Venturia, Verticillium, Pyricularia, Magnaporthe, Plasmopara, Pythium, Sclerotinia, Colletotrichum, Penicillium, Neurospora, Aspergillus, or Ashbya.

Throughout the various embodiments, the introduction into a cell according to step (b) of the tenth aspect may be achieved by any suitable method known in the art. The skilled person is well aware that a variety of different transformation or transfection (used interchangeably herein) techniques are available depending on the desired target cell. Introduction may comprise methods, such as but not limited to calcium-phosphate-mediated transfection, catioinic-polymer-mediated transfection, liposome-mediated transfection, PEG-mediated transfection, dendrimer transfection, heat shock transfection, magnetofection, electroporation, particle, including nanoparticle, uptake or bombardment, or microinjection.

In embodiments in which the cell is a plant cell, introduction into the plant cell may be a method such as, but not limited to, particle bombardment, particle uptake, whiskers mediated transformation, Agrobacterium transformation, including Agrobacterium-mediated introduction of virus-based vectors, PEG-mediated transformation, liposome-mediated transformation, electroporation, cell-penetrating peptides, microinjection or viral-vector-mediated introduction. As the skilled person is well aware, for some introduction techniques, for example PEG-mediated transformation, liposome-mediated transformation, electroporation or cell-penetrating peptides, the plant cell wall may be removed to produce protoplasts prior to the introduction. In embodiments comprising introduction into at least one protoplast, step (g) of the method of the tenth aspect may comprise regeneration from the at least one protoplast.

In embodiments, in which the cell is a fungal cell, including a yeast cell, introduction into the fungal cell, including a yeast cell, may comprise partial or complete digestion of the cell wall and/or may comprise protoplast transformation.

In some embodiments, the introduction comprises nuclear transformation. In some embodiments, the introduction comprises nuclear plastid transformation, such as chloroplast or mitochondrial transformation.

In one embodiment of the various aspects disclosed herein, the modification may be at least one insertion, at least one deletion, or at least one point mutation.

In one embodiment of the tenth aspect, during step (a) to (c), at least one additional effector, or a nucleic acid sequence encoding the same, may be provided, the additional effector promoting DNA repair and cell regeneration, or another activity before, during or upon insertion of at least one nick at the genomic locus of interest at or near at least one target site. The additional effector, may be selected from, but is not restricted to, at least one additional effector having an enzymatic activity that modifies at least one target nucleic acid (e.g., nuclease activity, e.g. exonuclease activity, methyltransferase activity, demethylase activity, DNA repair activity, DNA damage activity, deamination activity, dismutase activity, alkylation activity, depurination activity, oxidation activity, pyrimidine dimer forming activity, helicase activity (e.g. SF1/2, SF3, SF4), integrase activity, telomerase activity, topoisomerase activity, e.g. gyrase activity, transposase activity, transcriptase or reverse transcriptase activity, recombinase activity, polymerase activity, e.g. RNA polymerase activity or DNA polymerase activity e.g. Pol theta activity, ligase activity, photolyase activity or glycosylase activity).

In one embodiment of the tenth aspect, the method may be a concerted double-nicking method, wherein at least two Cas enzymes having nickase activity (nCas), or catalytically active fragments thereof, or at least one nucleic acid sequence encoding the same, are provided in step (b); and wherein in step (c) at least two compatible guide RNAs are provided, wherein the at least two compatible guide RNAs are designed to allow a concerted action of the at least two Cas enzymes having nickase activity so that the at least two Cas enzymes having nickase activity introduce two individual nicks at the at least one target site.

In one embodiment, the two Cas enzymes having nickase activity, or the catalytically active fragments thereof, can be the same or different, wherein at least one of the at least two Cas enzymes having nickase activity, or the catalytically active fragment thereof, is an engineered Cas12a enzyme having nickase activity (nCas12a), or a catalytically active fragment thereof, or the sequence encoding the same, as defined in any one of claims 1 to 6, wherein the nCas12a can be the same nCas12a, or a different nCas12a.

In certain embodiments, the two individual nicks are in close enough proximity to cause a DSB. In other embodiment, the two individual nicks do not lead to a DSB (cf. WO2021122080A1).

In one embodiment, the two individual nicks may be introduced into opposite strands within the genomic locus of interest of the at least one cell or construct at or near the at least one target site, wherein the offset is positive, negative, or zero, preferably wherein the offset is between around −100 bp and +100 bp.

In certain embodiments the offset may be negative, preferably wherein the offset is −40 bp to −30 bp, or 30 bp to −20 bp, or 20 bp to −10 bp, or 10 bp to −1 bp.

In other embodiments, the offset may be positive, preferably wherein the offset is 1 bp to 10 bp, or 10 bp to 20 bp, or 20 bp to 30 bp, or 30 bp to 40 bp, or 40 bp to 50 bp, or 50 bp to 60 bp, or 60 bp to 70 bp, or 70 bp to 80 bp, or 80 bp to 90 bp, or 90 bp to 100 bp, more preferably wherein the offset is 20 bp to 40 bp, most preferably wherein the offset is 25 bp to 35 bp.

In one embodiment, the two Cas enzymes having nickase activity and/or the at least two compatible guide RNAs are individually provided in the form of at least one expression construct or vector, or in the form of at least one complex, or in the form of at least one nucleic acid sequence encoding the same, or in the form of at least one fusion protein or at least one nucleic acid sequence encoding the same.

In one embodiment, the at least one cell or construct originates from a prokaryotic cell, including a bacterial or an archaea cell, or a eukaryotic cell.

In certain embodiments, the cell is a plant cell, including an algal cell, preferably wherein the cell may be selected from a cell originating from a plant which belongs to the superfamily Viridiplantae, in particular monocotyledonous and dicotyledonous plants including but not limited to fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

Preferred plants are Abelmoschus spp., Allium spp., Apium graveolens, Asparagus officinalis, Avena spp. (e.g. Avena sativa, Avena fatua, Avena byzantina, Avena fatua var. sativa, Avena hybrida), Beta vulgaris, Brassica spp. (e.g. Brassica napus, Brassica rapa ssp. [canola, oilseed rape, turnip rape]), Capsicum spp., Citrullus lanatus, Cucumis spp., Cynara spp., Daucus carota, Glycine spp. (e.g. Glycine max, Soja hispida or Soja max), Gossypium hirsutum, Helianthus spp. (e.g. Helianthus annuus), Hordeum spp. (e.g. Hordeum vulgare), Lactuca sativa, Medicago sativa, Oryza spp. (e.g. Oryza sativa, Oryza latifolia), Pennisetum sp., Saccharum spp., Secale cereale, Solanum spp. (e.g. Solanum tuberosum, Solanum integrifolium or Solanum lycopersicum), Sorghum bicolor, Spinacia spp., Triticum spp. (e.g. Triticum aestivum, Triticum durum, Triticum turgidum, Triticum hybernum, Triticum macha, Triticum sativum, Triticum monococcum or Triticum vulgare), or Zea mays.

Preferred plants, in certain embodiments, may also be selected from Brassica spp. (e.g. Brassica napus, Brassica rapa ssp. [canola, oilseed rape, turnip rape]), Capsicum spp., Glycine spp. (e.g. Glycine max, Soja hispida or Soja max), Gossypium hirsutum, Helianthus spp. (e.g. Helianthus annuus), Oryza spp. (e.g. Oryza sativa, Oryza latifolia), Solanum spp. (e.g. Solanum tuberosum, Solanum integrifolium or Solanum lycopersicum), Triticum spp. (e.g. Triticum aestivum, Triticum durum, Triticum turgidum, Triticum hybernum, Triticum macha, Triticum sativum, Triticum monococcum or Triticum vulgare), or Zea mays.

In other embodiments, the cell is a fungal cell, including a yeast cell, preferably wherein the fungal cell, including the yeast cell, is selected from a cell originating from to Saccharomyces spec, such as Saccharomyces cerevisiae, Hansenula spec, such as Hansenula polymorpha, Schizosaccharomyces spec, such as Schizosaccharomyces pombe, Kluyveromyces spec, such as Kluyveromyces lactis and Kluyveromyces marxianus, Yarrowia spec, such as Yarrowia lipolytica, Pichia spec, such as Pichia methanolica, Pichia stipites and Pichia pastoris, Zygosaccharomyces spec, such as Zygosaccharomyces rouxii and Zygosaccharomyces bailii, Candida spec, such as Candida boidinii, Candida utilis, Candida freyschussii, Candida glabrata and Candida sonorensis, Schwanniomyces spec, such as Schwanniomyces occidentalis, Arxula spec, such as Arxula adeninivorans, Ogataea spec such as Ogataea minuta, Ashbya spec, such as Ashbya gossypii, Aspergillus spec. such as Aspergillus niger or Myceliophthora thermophila.

In preferred embodiments, the cell is a eukaryotic cell or a prokaryotic cell, wherein the cell is selected from a cell originating from Rhodococcus rhodochrous, Aerococcus sp., Aspergillus sp., Bacillus pumilus, Bacillus subtilis, Bacteroides thetaiotaomicron, Clostridium algidicarnis, Corynebacterium efficiens, Corynebacterium glutamicum, Escherichia coli, Haloferax volcanii, Lactobacillus casei, Methanocaldococcus jannaschii, Methanothermobacter thermautotrophicus, Myceliophthora thermophila, Pichia pastoris, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Pseudozyma tsukubaensis, Ralstonia eutropha, Rhodobacter sphaeroides, Rhodococcus opacus, Saccharomyces cerevisiae, Shigella boydii, Sinorhizobium meliloti, Streptomyces antibioticus, Streptomyces avermitilis, Streptomyces cacaoi, Streptomyces coelicolor, Streptomyces flavelus, Streptomyces griseolus, Streptomyces lavendulae, Streptomyces lividans, Streptomyces olivaceus, Streptomyces tanashiensis, Streptomyces virginiae, Streptomyces viridochromogenes, Thermoplasma acidophilum, Vibrio natrigens or Yarrowia lipolytica, wherein the cell is prefererably selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica.

In certain embodiments, the cell is a eukaryotic cell or a prokaryotic cell, wherein the cell may be selected from a cell originating from Bacillus subtilis, Corynebacterium glutamicum, Escherichia coli, Pseudomonas aeruginosa, Pseudomonas putida, Rhodobacter sphaeroides, Rhodococcus opacus, Saccharomyces cerevisiae and Yarrowia lipolytica, Phakopsora spec, e.g. Phakopsora pachyrhizi, Zymoseptoria spec, e.g. Zymoseptoria tritici, Septoria, Mycosphaerella, Phythopthora spec., e.g. Phytopthora infestans, Puccinia, Sphaerotheca, Blumeria, Erysiphe, Alternaria, Botrytis, Ustilago, Venturia, Verticillium, Pyricularia, Magnaporthe, Plasmopara, Pythium, Sclerotinia, Colletotrichum, Penicillium, Neurospora, Aspergillus, or Ashbya.

In certain embodiments according to the various aspects herein, mismatches between the guide RNA and the target strand, for instance 1, 2, 3 or 4 mismatches, may favour nicking events. Without wishing to be bound by theory, it is hythesized that mutants with reduced flexibility, as for instance achieved by substitutions with proline, together with target DNA mismatches are sufficient to limit conformational changes and block target strand cleavage.

In an eleventh aspect, there is provided an edited cell, tissue, organ, material or whole organism obtained by or obtainable by a method according to the tenth aspect as disclosed.

In certain embodiments, the edited cell, tissue, organ, material or whole organism is not a plant or animal edited cell, tissue, organ, material or whole organism exclusively obtained by means of an essentially biological process.

The twelfth aspect relates to the use of a compound selected from (i) to (vi): (i) at least one engineered Cas12a enzyme having nickase activity (nCas12a), or a catalytically active fragment thereof, or at least one nucleic acid sequence encoding the same, as defined in the first aspect of the present invention; (ii) at least one expression construct or vector as defined in the third aspect of the present invention; or (iii) at least one complex or at least one nucleic acid sequence encoding the same as defined in the fifth aspect of the present invention, or a fusion protein or at least one nucleic acid sequence encoding the same as defined in the sixth aspect of the present invention; or (iv) at least one adenine or a cytidine base editor, or at least one base editor complex, or at least one nucleic acid sequence encoding the same as defined in the seventh aspect of the present invention; or (v) at least one prime editor or at least one prime editor complex, or at least one nucleic acid sequence encoding the same as defined in the eighth aspect of the present invention; or (vi) a kit as defined in the ninth aspect of the present invention; for introducing a nucleotide deletion or insertion or modification in a nucleic acid molecule, preferentially in a genome, including uses for optimizing or modifying a trait in a plant, including the modification of a yield-related trait, or a disease-resistance related trait, and/or for metabolic engineering in cell, including a prokaryotic or eukaryotic cell, preferably in a plant cell, an algal cell, a fungal cell, including a yeast cell, or an archaea cell.

Optimizing or modifying a trait in a plant may for instance comprise genetic modification leading to the comprisal of an endogenous gene or a transgene that confers herbicide resistance, such as the bar or pat gene, which confer resistance to glufosinate ammonium (Liberty®, Basta® or Ignite®; EP0242236 and EP0242246); or any modified EPSPS gene, such as the 2mEPSPS gene from maize (EP0508909 and EP0507698), or glyphosate acetyltransferase, or glyphosate oxidoreductase, which confer resistance to glyphosate (RoundupReady®), or glyphosate resistant EPSPS, such as a CP4 EPSPS, or such as an N-acetyltransferase (gat) gene, or bromoxynitril nitrilase to confer bromoxynitril tolerance, or any modified AHAS gene, which confers tolerance to sulfonylureas, imidazolinones, sulfonylaminocarbonyltriazolinones, triazolopyrimidines or pyrimidyl(oxy/thio)benzoates, such as oilseed rape imidazolinone-tolerant mutants PM1 and PM2, currently marketed as Clearfield® canola; and/or an endogenous gene or a transgene that confers increased oil content or improved oil composition, such as a 12:0 ACP thioesteraseincrease to obtain high laureate, which confers pollination control, such as barnase under control of an anther-specific promoter to obtain male sterility, or barstar under control of an anther-specific promoter to confer restoration of male sterility, or such as the Ogura cytoplasmic male sterility and nuclear restorer of fertility; and/or an endogenous gene or a transgene that confers resistance to glufosinate ammonium (Liberty®, Basta® or Ignite®); and/or a gene coding for a phosphinothricin-N-acetyltransferase (PAT) enzyme, such as a coding sequence of the bialaphos resistance gene (bar) of Streptomyces hygroscopicus. Such plants may, for example, comprise the elite events MS-BN1 and/or RF-BN1 as described in WOO1/41558, or elite event MS-B2 as described in WOO1/31042, or any combination of these events.

Examples of technically induced mutants in Brassica napus, as a result of optimizing of modifying a trait, are mutants in the FATB gene as described in WO2009007091 or in the FAD3 genes as described in WO2011/060946, or may be podshatter resistant mutants such as mutants described in WO2009068313 or in WO2010006732, or mutations conferring herbicide tolerance such as the PM1 and PM2 mutations conferring imidazolinone tolerance (Tan et al. 2005; U.S. Pat. No. 5,545,821).

In one embodiment of the twelfth aspect, the use comprises a paired nickase strategy as defined in the second aspect disclosed herein.

In a thirteenth aspect, there is provided a method of treating or preventing a disease, the method comprising using (i) at least one engineered Cas12a enzyme having nickase activity (nCas12a), or a catalytically active fragment thereof, or at least one nucleic acid sequence encoding the same, as defined in the first aspect of the present invention; (ii) at least one expression construct or vector as defined in the third aspect of the present invention; or (iii) at least one complex or at least one nucleic acid sequence encoding the same as defined in the fifth aspect of the present invention, or a fusion protein or at least one nucleic acid sequence encoding the same as defined in the sixth aspect of the present invention; or (iv) at least one adenine or a cytidine base editor, or at least one base editor complex, or at least one nucleic acid sequence encoding the same as defined in the seventh aspect of the present invention; or (v) at least one prime editor or at least one prime editor complex, or at least one nucleic acid sequence encoding the same as defined in the eighth aspect of the present invention; or (vi) a kit as defined in the ninth aspect of the present invention; or (vii) a cell as defined in the fourth aspect of the present invention; or (viii) an edited cell, tissue, organ, material or whole organism as defined in the eleventh aspect of the present invention; for introducing at least one modification in a genomic locus of interest of at least one cell of a subject in need thereof at or near at least one disease-state related target site.

In one embodiment, the method may comprise an ex vivo modification of the genomic locus, wherein at least one cell of a subject is provided to perform an ex vivo modification of the genomic locus to obtain at least one edited cell.

In a fourteenth aspect, there is provided a compound selected from: (i) at least one engineered Cas12a enzyme having nickase activity (nCas12a), or a catalytically active fragment thereof, or at least one nucleic acid sequence encoding the same, as defined in the first aspect of the present invention; (ii) at least one expression construct or vector as defined in the third aspect of the present invention; or (iii) at least one complex or at least one nucleic acid sequence encoding the same as defined in the fifth aspect of the present invention, or a fusion protein or at least one nucleic acid sequence encoding the same as defined in the sixth aspect of the present invention; or (iv) at least one adenine or a cytidine base editor, or at least one base editor complex, or at least one nucleic acid sequence encoding the same as defined in the seventh aspect of the present invention; or (v) at least one prime editor or at least one prime editor complex, or at least one nucleic acid sequence encoding the same as defined in the eighth aspect of the present invention; or (vi) a kit as defined in the ninth aspect of the present invention; or (vii) a cell as defined in the fourth aspect of the present invention; or (viii) an edited cell, tissue, organ, material or whole organism as defined in the eleventh aspect of the present invention; for use in a method of treating or preventing a disease in a patient.

The fifteenth aspect relates to the use of a compound selected from (i) at least one engineered Cas12a enzyme having nickase activity (nCas12a), or a catalytically active fragment thereof, or at least one nucleic acid sequence encoding the same, as defined in the first aspect of the present invention; (ii) at least one expression construct or vector as defined in the third aspect of the present invention; or (iii) at least one complex or at least one nucleic acid sequence encoding the same as defined in the fifth aspect of the present invention, or a fusion protein or at least one nucleic acid sequence encoding the same as defined in the sixth aspect of the present invention; or (iv) at least one adenine or a cytidine base editor, or at least one base editor complex, or at least one nucleic acid sequence encoding the same as defined in the seventh aspect of the present invention; or (v) at least one prime editor or at least one prime editor complex, or at least one nucleic acid sequence encoding the same as defined in the eighth aspect of the present invention; or (vi) a kit as defined in the ninth aspect of the present invention; or (vii) a cell as defined in the fourth aspect of the present invention; or (viii) an edited cell, tissue, organ, material or whole organism as defined in the eleventh aspect of the present invention; for use in in the manufacture of a medicament for treating or preventing a disease in a patient.

All methods disclosed herein exclude processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes, optionally, where the method comprises the following step: (g) regenerating at least one population of edited cells, tissues, organs, materials or whole organisms from the at least one edited cell or construct.

According to the various aspects and embodiments disclosed herein relating to a compound selected from (i) at least one engineered Cas12a enzyme having nickase activity (nCas12a), or a catalytically active fragment thereof, or at least one nucleic acid sequence encoding the same, as defined in the first aspect of the present invention; (ii) at least one expression construct or vector as defined in the third aspect of the present invention; or (iii) at least one complex or at least one nucleic acid sequence encoding the same as defined in the fifth aspect of the present invention, or a fusion protein or at least one nucleic acid sequence encoding the same as defined in the sixth aspect of the present invention; or (iv) at least one adenine or a cytidine base editor, or at least one base editor complex, or at least one nucleic acid sequence encoding the same as defined in the seventh aspect of the present invention; or (v) at least one prime editor or at least one prime editor complex, or at least one nucleic acid sequence encoding the same as defined in the eighth aspect of the present invention; or (vi) a kit as defined in the ninth aspect of the present invention; or (vii) a cell as defined in the fourth aspect of the present invention; or (viii) an edited cell, tissue, organ, material or whole organism as defined in the eleventh aspect of the present invention, the compound is provided in a functional form, e.g., including stabilizers, cofactors, means for introducing the same into a target cell or tissue and the like.

### EXAMPLES

### Example 1: Rational Protein Design

One major approach in the generation of Cas12a mutants with in vivo nickase activity was rational protein design. This approach is on one part based on data available in the literature describing Cas12a mutants that have at least partial and/or at least in vitro nickase activity. Mutants that were used as basis for rational protein design were LbCas12a R1338A (Yamano et al., 2017; \hat{=} FnCas12a R1218A), and FnCas12a K1013G/R1014G (WO 2019/233990; =LbCas12a K932G/N933G).

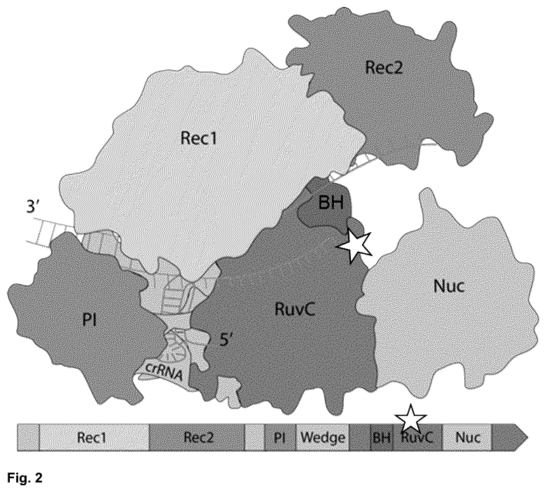

Secondly, rational protein design is based on crystal structure information of Cas12a as well as available mechanistic insight of the cleavage event. In contrast to Cas9, where the RuvC and the HNH domains each cleave one strand, the RuvC domain of Cas12a cleaves both the non-target strand (NTS) and the target strand (TS) sequentially. In general, rational design approach focused on mutating the so-called lid of the RuvC domain, which is located next to the active site of the RuvC domain and has—so far—not attracted much attention for the generation of Cas12a nickase mutants. The lid opens and closes, to provide access to the active site and may have a role in the transition (after NTS cleavage) towards the second cleavage event. This strategy focuses on mutating the core lid domain as defined in SEQ ID NO: 13 (see FIG. 1) and avoids mutating the catalytic residue E925 (LbCas12a) so that the catalytic center of the RuvC domain is not inactivated completely. All mutations were introduced by standard cloning methods. See FIG. 2 for Cas12a domain architecture.

### Example 2: Targeted in Silico Analysis

To provide a basis for expanding the rational protein design and in vitro and in vivo screens to all Cas12a variants described as effective in genome editing, and available in databases, and further, of course, to those Cas12a sequences available, yet not annotated, a systematic in silico screen and comparison was set up. The aim was to define a suitable consensus motif applicable for all Cas12a enzymes described and yet to be described to reasonably expand the scope of the nickase design. To this end, BLAST protein searches (NCBI; https://blast.ncbi.nlm.nih.gov/Blast.cgi?PAGE=Proteins; standard parameters) were performed to get an overview of Cas12a/Cpf1 enzymes with known functions and closely related Cas12a enzymes with presently unknown function. Notably, all enzymes showed very high sequence conservation in the region corresponding to the lid domain as described for, for example, LbCas12a and AsCas12a. In addition, there was a high overall sequence identity/homology in the sequences screened. Therefore, it was assumed that the findings obtained for Cas12a enzymes studied herein could be easily transferred to other Cas12a enzymes.

Next, after completing the searches with BLAST using heuristic algorithms, multiple sequence alignments using seeded guide trees and HMM profile-profile techniques to generate alignments between three or more sequences were performed with Clustal Omega (EMBL-EBI; again using standard parameters) by aligning certain sequences of Cas12a enzymes analyzed herein and disclosed to be suitable in genome editing in various settings (provided as SEQ ID NOs: 1 to 12). As shown in FIG. 1, there is a high degree of sequence conservation within the alpha2/beta6 and the alpha3 domain described for AsCas12a and LbCas12a by structural analyses (cf. Stella et al., 2018, Suppl. FIG. S4). Particularly strong sequence conservation was observed in the domain (with reference to LbCas12a/SEQ ID NO: 1 in FIG. 1) starting on L927 of Cas12a. As this position was fully conserved in all sequences analyzed, this position was defined as the starting position of the so-called core lid domain as used herein. Most of the Cas12a sequences (only exception AsCas12a) had an identical length within the core lid domains. The end position of the core lid domain was thus defined as position V942 in LbCas12a (SEQ ID NO: 1) and as V1011 of AsCas12a (SEQ ID NO: 2). It should be noted that, for example, for Francisella tularensis (and various subspecies thereof, including novicida, including U112) several Cas12a variants have been described. Variants can be easily identified via NCBI taxonomy browser search and sequence databases. As the alignments of five different Cas12a variants performed for Cas12a enzymes within the Francisella genus revealed that these were completely identical in their core lid domain consensus sequence (for example, FIG. 1 for SEQ ID NOs: 3 and 4 as two exemplary sequences), only two Francisella sequences were included in the further alignments and it was decided to rather include Cas12a variants from different origins to have a reliable result on the degree of conservation of a potential lid consensus sequence over a variety of different species. As can be derived from FIG. 1, the relevant consensus sequence for a core lid domain motif and the position thereof in a Cas12a enzyme can be easily defined. A core lid domain motif was then defined and used as basis for further targeted protein design studies (cf. SEQ ID NO: 13), as it could be shown that this motif had indeed a high degree of conservation and could thus serve as identifier or consensus sequence for a highly conserved region within Cas12a enzymes.

To further demonstrate that the core lid domain motif (cf. SEQ ID NO: 13) was a helpful new structural motif to generalize the findings for LbCas12a, AsCas12a and other variants as studied to any kind of homologous Cas12a enzyme, additional analyses were performed. To this end, MUSCLE (EMBL-EBI; MUltiple Sequence Comparison by Log-Expectation; default parameters) was used to align Cas12a sequences (here: SEQ ID NO: 1 to 12). Corroborating our previous findings, MUSCLE alignments confirmed that the core lid motif (SEQ ID NO: 13) as chosen is a suitable identifier to characterize Cas12a variants of many species (homologs, orthologs, paralogs), as the motif as defined is highly conserved amongst the various variants. To finally confirm that the core lid domain was a suitable structural motif to characterize a Cas12a enzyme, along with the overall sequence identity/homology derivable from data bases (primary amino acid sequence) and the structural characteristics on a three-dimensional level known for certain Cas12a enzymes, a further analysis (based on the MUSCLE alignment of SEQ ID NO: 1 to 12) was performed (using: MView; version 1.63; default parameters see setting: https://www.ebi.ac.uk/seqdb/confluence/display/JDSAT/MView+Help+and+Documentation). MView, using AsCas12a (SEW ID NO:2) with the longest core lid domain as reference sequence along with other Cas12a variants (SEQ ID NOs: 1, 3 to 12) allowed the calculation of percentages of coverage (cov) and identity (pid) for consensus sequences of 100%, 90%, 80% and 70%. Based on this finding, a core lid domain consensus sequence was constructed (now: SEQ ID NO: 13) and it was used, in an iterative way, for alignment purposes. First, BLAST protein searches for Cas12a variants were performed, then a sub-search for the presence of the core lid domain consensus was performed. Together, these analyses confirmed that the core lid domain as defined during the project is indeed a highly conserved signature motif and represents a valuable consensus sequence to identify and characterize Cas12a enzymes.

Interestingly, new insights into the mechanism for target recognition and cleavage by other Cas12 endonucleases demonstrated that the core lid domain is also structurally conserved in Cas12i, Cas12b and Cas12e, although the protein sequences of the lid region in these Cas12 orthologs are highly divergent (cf. Zhang et al., 2018, Extended Data FIG. 8). Because of this structural conservation, the core lid domain may also constitute an interesting motif providing novel opportunities to improve and expand genome editing applications of class II, type V enzymes other than Cas12a.

### Example 3: In Vivo Screening Assay for Cas12a Nickase Candidates

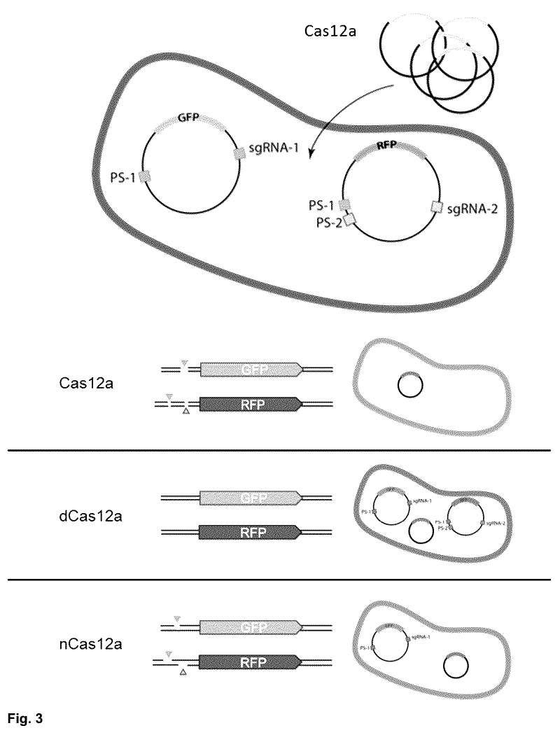

An in vivo assay for different types of Cas nickases has been developed that consists of a 3-plasmid system: two reporter plasmids are used and a third Cas encoding plasmid. The reporter plasmids consist of a GFP-encoding plasmid that encodes guide RNA 1 and carries target-1 flanked by the appropriate PAM motif. The second plasmid is an RFP-encoding plasmid which encodes 2 guide RNAs and carries overlapping target-1 and target-2, each with the appropriate PAM motif. Upon transformation of the Cas-encoding plasmid into a cell hosting the two reporter plasmids (in the absence of antibiotic selection for the two reporter plasmids, but in the presence of a selective antibiotic for the Cas-encoding plasmid), the red/green fluorescence readout produces a distinctive phenotype for a nickase, wild-type or dead Cas nuclease. Nuclease activity results in loss of both GFP and RFP, while nickase activity will only disrupt RFP, due to double nicking on the two overlapping target sites, but not GFP as there is only one target site to be nicked. Catalytically inactive Cas12a variants will result in both RFP and GFP fluorescence (see FIG. 3).

The in vivo screening assay was originally established and optimized using a Cas9 nuclease, Cas9 DH10A and Cas9 H840A nickases, and a dead Cas9 to verify correct readouts of the assay. Upon establishing and validating the reporter assay with Cas9, it was used for testing LbCas12a candidate nickases, either in single genotype experiments (one-by-one) or in a high throughput manner using fluorescence-activated cell sorting (FACS).

The following plasmids were created for the Cas12a in vivo nicking assay: pGFP (SEQ ID NO: 52; pSC101 RepA N99D, KanR; GFP under PlaclQ promoter; target-1; Cas12a guide RNA 1 under PJ23119 promoter); pRFP (SEQ ID NO: 53; pBR322 AmpR; RFP under Amp (Bla) promoter; Target-1; Target-2, Cas12a guide RNA 2 under PJ23119 promoter); pCas LbCas12a WT (SEQ ID NO: 54; p15A (pCB482), CamR; LbCas12a under PJ23108 promoter; encodes SEQ ID NO:1), pCas LbCas12a dead (SEQ ID NO: 55; p15A (pCB482), CamR; LbCas12a dead under PJ23108 promoter; encodes LbCas12a E925A/D832A (mutation relates to reference sequence SEQ ID NO: 1)).

To produce the rationally designed Cas12a variants, point mutations were introduced into the pCas LbCas12a WT template (SEQ ID NO: 53). Inverse PCR site-directed mutagenesis was used to introduce the mutations using 5′ phosphorylated primers that contain the desired mutations at the 5′ end of its sequence. Different primers sets were designed according to the variant to produce.

In a first experiment, individual LbCas12a variants were introduced into the E. coli GFP/RFP reporter strain (DH10b). After the individual (one-by-one) transformation of the LbCas12a variant (10ng) by heat shock, the transformed cells were recovered in 950 μl of LB medium for 1 hour and then 2 μl of the recovered transformation inoculated in 200 μl of M9TG media containing Chloramphenicol [35 mg/I] and incubated at 37° C. overnight (day 1). On the following day (day 2), a 1:10,000 times dilution was reinoculated in 200 μl of fresh M9TG media containing Chloramphenicol [35 mg/I] and incubated at 37° C. overnight. After 20 hours the produced cultures were diluted in 1×PBS (1:10 dilution), and the green and red fluorescence of the samples were measured in a plate reader.

Results of some selected variants, mutated in the RuvC lid, are shown in FIG. 4. LbCas12a S934A/R935G (mutation relates to reference sequence SEQ ID NO: 1) and LbCas12a K932G/N933G (mutation relates to reference sequence SEQ ID NO: 1; this double mutant is the LbCas12a homologue of the previously reported FnCas12a K1013G/R1014G mutant, WO 2019/233990) showed a wild-type-like nuclease activity and appeared to cleave both strands in vivo. In contrast, the LbCas12a quadruple mutant K932G/N933G/S934A/R935G (SEQ ID NO: 14) showed the desired nickase phenotype. The negative RuvC lid mutation (LbCas12a F931E/K932E/R935D/K937D/K940D, mutation relates to reference sequence SEQ ID NO: 1) appears to be a dead Cas12a. Likewise, the previously reported LbCas12a R1138A mutant showed a dead Cas12a phenotype.

### Example 4: Laboratory Evolution—Semi-Random RuvC Lid Mutagenesis

As described above, the aim of the present invention is the provision of a robust nickase variant of LbCas12a. Apart from the aforementioned rational design (Examples 1 and 3), laboratory evolution approaches were performed in parallel. Laboratory evolution is an extremely powerful approach for optimizing protein functionality in an unbiased manner. An essential requirement of laboratory evolution is coupling of the genotype (gene encoding desired Cas12a variant) to the phenotype (desired Cas12a functionality, in this case: efficient dsDNA nicking). This was achieved by transforming the GFP/RFPP E. coli strain (see example 3) with a library of Cas12a variants and selecting green-fluorescent transformants—either manually or using Fluorescence-Activated Cell Sorting (FACS).

As the Cas12a RuvC lid quadruple mutant (LbCas12a K932G/N933G/S934A/R935G, SEQ ID NO: 14) showed a reduced GFP signal compared to dead LbCas12a (see example 3 and FIG. 4), semi-random saturation mutagenesis was undertaken in an attempt to further improve nickase activity of this variant. We randomly substituted amino acid residues 931 to 940 (10 residues, residues refer to positions in SEQ ID NO: 1 and correspond to positions 5 to 15 of SEQ ID NO: 13, note that SEQ ID NO: 13 has one optional position that is not present in LbCas12a) using degenerated NNK codons (N=A,C,G,T; K=G,T), this codon motif codes for the 20 different canonical amino acids and a single stop codon. This design aims to replace the entire portion of the lid with random amino acids, including the wild-type residues. [0223] pCas Lbl2a WT (SEQ ID NO: 53) was ‘opened’ at positions coding for G930 and Q941 using a pair of primers containing a 5′ SapI restriction site. The digested PCR product was then ligated (T4 DNA ligase) using as insert two short complementary oligos which upon annealing form an overhang complementary to the overhangs left by the SapI nuclease. The insert oligos contain degenerated NNK nucleotides, which upon correct assembly of the constructs, generate a library of plasmids coding for LbCas12a with different coding sequences at the oligos insertion site.

The resulting RuvC Lid NNK library was then introduced into the E. coliGFP/RFP reporter strain (Examples 3 and 4). The culture generated after transformation was diluted and plated on media selecting for the Cas12a encoding plasmid (Chloramphenicol [50 mg/L]). GFP⁺/RFP⁻ (green) cells are expected in case of a LbCas12a nickase. Single green colonies in the plate were selected for Sanger sequencing to retrieve the LbCas12a genotype inside the green fluorescent phenotypical colonies. The retrieved single genotype variants were then re-introduced into the E. coli GFP/RFP reporter strain individually to validate nicking activity based on the fluorescence signal readout from each culture/variant (i.e. individual LbCas12a sequences isolated from the population).

Manual selection of green colonies
(i) DH10b chemically competent cells containing the pGFP pRFP reporter plasmids were transformed with 500ng (˜100 fmol) of the RuvC Lid NNK library. Transformed cells were recovered in 950 μl of LB medium for 1 hour at 37° C. After recovery, the recovered transformation was aliquoted into 50 ml of LB medium and incubated overnight (ON) at 37° C.
(ii) On the next day (day 2), a 1:10,000 dilution was plated on LB agar+Chloramphenicol [50 mg/L] and incubated ON at 37° C.
(iii) On the following day (day 3), the plates were removed from the fridge and placed for ˜5 hours at 4° C. (fluorophore maturation). The plates were visualized under a blue light and screened for green colonies. Single green colonies were transferred (re-streaked) into a fresh plate containing Lb agar+Chloramphenicol [50 mg/L] and incubated ON at 37° C.
(iv) On the following day (day 4), the re-streak plate was replicated (each streak transferred to a new media) into a plate containing LB agar+Chloramphenicol [50 mg/L] and incubated ON at 37° C.
(v) On the following day (day 5), the plates were placed for ˜5 hours at 4° C. (fluorophore maturation) After the re-streak incubation, the plates were visualized under blue light to select green fluorescent colonies. Colonies displaying a green fluorescent phenotype were inoculated (N=32) independently in LB medium+Chloramphenicol [50 mg/L] and incubated ON at 37° C.
(vi) On the following day (day 6), the generated cultures were processed to extract plasmids (miniprep) and Sanger sequencing was used to reveal the sequence of the mutated region in the RuvC lid of each colony. The resulting sequencing information was processed in BenchLing to determine the sequence of each colony and for grouping based on the recurrence of sequences.

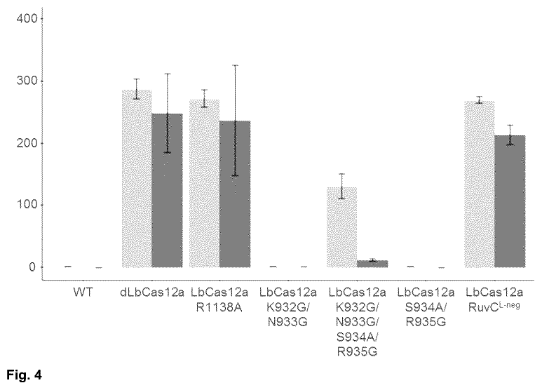

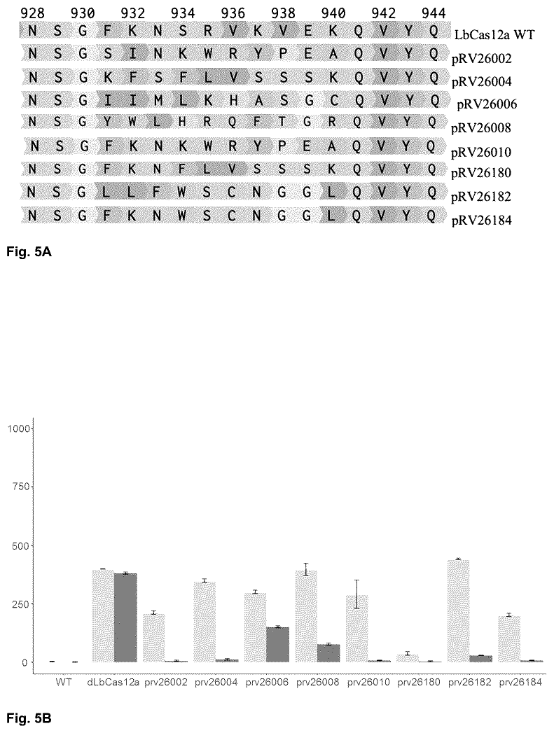

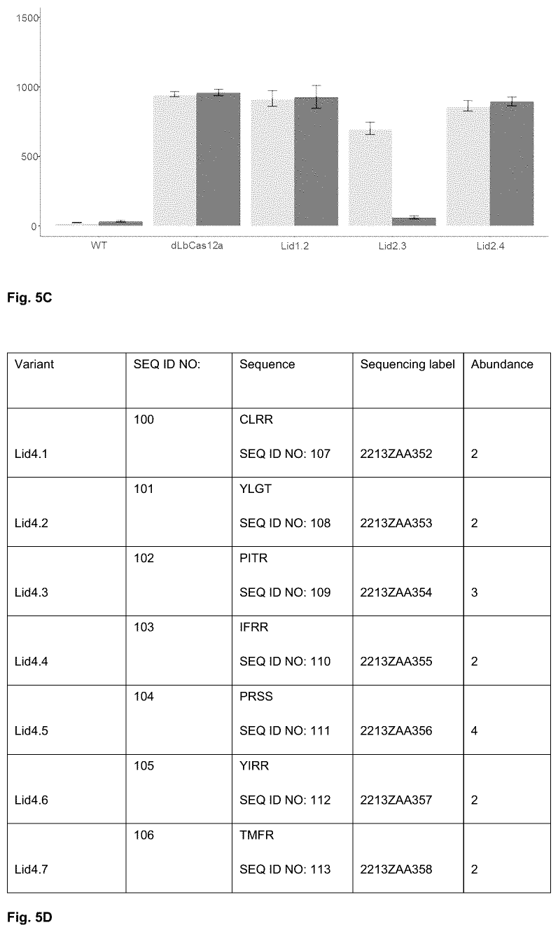

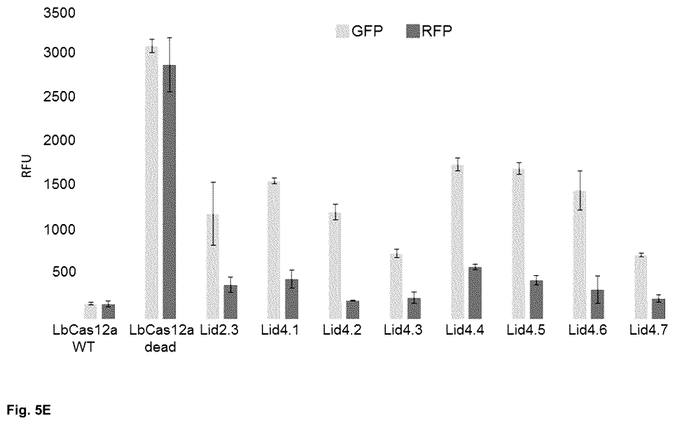

Exemplary GFP/RFP readouts of manually selected RuvC Lid variants (see FIG. 5A for the different core lid mutations) are shown in FIG. 5B.

Selection of green colonies by FACS
(i) DH10b chemically competent cells containing the pGFP and pRFP reporter plasmids were transformed with 500ng (˜100 fmol) of the RuvC Lid NNK library. Transformed cells were recovered in 950 μl of LB medium for 1 hour at 37° C. After recovery, the recovered transformation was aliquoted into 10 ml of LB medium and incubated overnight (ON) at 37° C.
(ii) On the following day (day 2), the cultures were diluted 40× times into sterile 1×PBS. One fraction of the culture was analyzed via flow cytometry (Day1, presorting) ON at 37° C. The produced sample was FACS sorted (First round of sorting). Cells displaying a strong GFP⁺ and RFP⁻ phenotype were collected in a separate tube containing 2 ml LB medium+Chloramphenicol [50 mg/L] and incubated ON at 37° C.
(iii) On the next day (day 3), the cultures were diluted 40× times into sterile 1×PBS. One fraction of the culture was analyzed via flow cytometry (Day 2, sorted once). Additionally, a 1:10,000 dilution of the culture was plated (10 μlates) on Lb agar+Chloramphenicol [50 mg/L] and incubated ON at 37° C. The produced sample was FACS sorted (Second round of sorting). Cells displaying a strong GFP⁺ and RFP⁻ phenotype were collected in a separate tube containing 2 ml LB medium+Chloramphenicol [50 mg/L] The collected cells were aliquoted to 10 ml LB medium+Chloramphenicol [50 mg/L] and incubated ON at 37° C.
(iv) On the next day (day 4) a 1:10,000 dilution of the culture was plated (10 μlates) on Lb agar+Chloramphenicol [50 mg/L] and incubated O/N at 37° C. One fraction of the culture was analyzed via flow cytometry (Day 3, sorted twice).
(v) On the following day (day 5), the plates were placed for ˜5 hours at 4° C. (fluorophore maturation). The plates were visualized under blue light to select green fluorescent colonies. Individual green colonies were transferred (re-streaked) onto fresh plates containing LB agar+Chloramphenicol [50 mg/L] and incubated ON at 37° C.
(vi) On the next day (day 6), the re-strike plate was replicated (each streak transferred to a new media) onto a plate containing LB agar+Chloramphenicol [50 mg/L] and incubated ON at 37° C.
(vii) On the following day (day 7), the plates were placed for ˜5 hours at 4° C. (fluorophore maturation). The plates were visualized under blue light to select green fluorescent colonies. Colonies displaying a green fluorescent phenotype were grown (N=12, n=6 per bio replicate) independently in LB medium+Chloramphenicol [50 mg/L] and incubated ON at 37° C.
(viii) On the next day (day 8), the generated cultures were processed to extract plasmids (miniprep) and Sanger sequencing was used to reveal the sequence of the mutated region of the RuvC Lid of each colony. The resulting sequencing information was processed in BenchLing to determine the sequence of each colony and for grouping based on the recurrence of sequences.

GFP/RFP results of exemplary mutants after FACS sorting are shown in FIG. 5C.

### Optimization of RuvC Lid Deletion Variant

A second round of site-directed saturation mutagenesis was undertaken to randomly substitute both the four amino acid residues (Y930, C931, S932 and S933) comprising the lid domain of a deletion variant identified in the first screen ((RuvCL-dell, SEQ ID NO: 15) as well as E925, a residue that is part of the highly conserved DED active site of Cas12a.

The diversity library was generated essentially as described above using insert oligos containing degenerated NNK nucleotides. The obtained plasmid population was Sanger sequenced to confirm correct assembly of the constructs and then transformed into the E. coli GFP/RFP reporter strain (see Examples 3 and 4). Following FACS sorting to enrich for GFP⁺/RFP⁻ cells, the sorted population was plated on chloramphenicol-containing media to select for the Cas12a-encoding plasmid and single green fluorescent colonies were selected for Sanger sequencing to retrieve the LbCas12a genotype and multiple sequence alignments listing all single genotype variants identified in the population were created (data not shown, all sequences for alignment presented in attached sequence listing). Interestingly, all variants obtained code for a glutamate at position 925, indicating that only cells containing catalytically active LbCas12a variants were sorted during the experiment. Moreover, while significant sequence variation was observed within the mutagenized lid region, the original deletion mutant ((RuvC^{L-del1}, SEQ ID NO: 15) was not found among the sampled colonies.

Although only 57 colonies were sequenced, several variants were identified multiple times (see FIG. 5D). These enriched variants (SEQ ID NO: 100 to SEQ ID NO: 106) were then re-introduced into the E. coli GFP/RFP reporter strain individually to validate nicking activity based on the fluorescence signal readout from each culture/variant. Plasmids encoding either wild-type LbCas12a (pRV060) or a catalytically dead variant (pRV061) were used as positive and negative controls while the original lid deletion variant (Lid2.3; SEQ ID NO: 15) was included to benchmark the nicking activity of the newly identified variants. FIG. 5E shows the normalized relative fluorescence units (fluorescence/OD600, average of three biological replicates). Interestingly, multiple variants display either enhanced GFP expression or a lower RFP signal compared to the original Lid2.3 mutant, which is suggestive of enhanced nickase activity and/or reduced residual DSB activity. In vitro validation of variants recovered from RuvC Lid NNK library

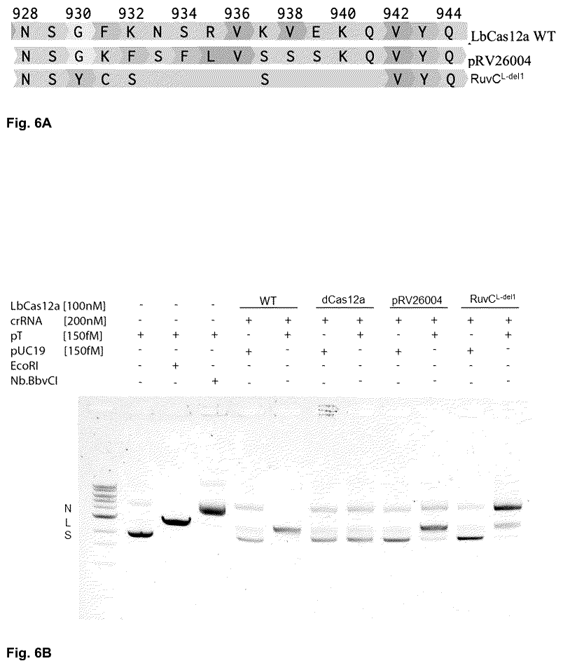

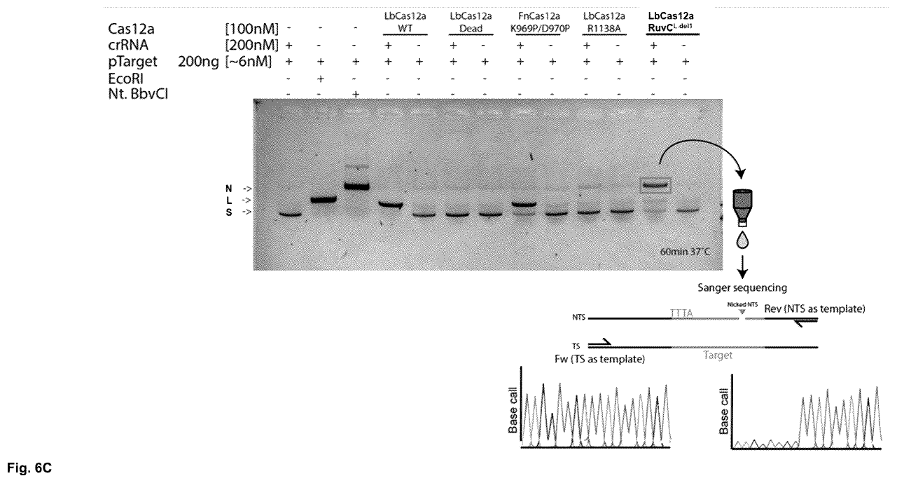

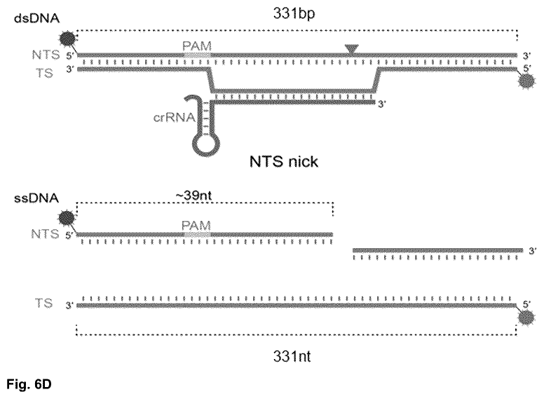

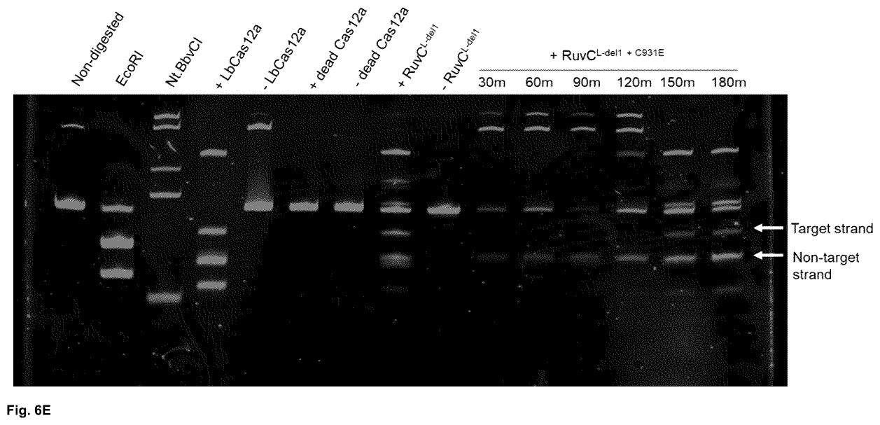

Lid variant pRV26004 (SEQ ID NO: 16) and a version of a lid deletion variant (RuvC^{L-del1}, SEQ ID NO: 15) (see FIG. 6A), as well as wild type and dead LbCas12a were used for in vitro validation. The selected LbCas12a variants were cloned in a pET (pML-1B, KanR. Addgene #29653) vector, including a 6×Histidine tag at the N terminus of the protein.

The vectors encoding the selected variants were introduced into E. coli Rosetta DE3 competent cells (each variant individually). A single colony from each transformed variant was used to inoculate 10 ml LB medium containing Chloramphenicol [50 mg/I] and Kanamycin [35 mg/I] and incubated at 37° C. overnight. On the next day, the overnight culture of each variant was used to inoculate 250 ml LB medium containing Chloramphenicol [50 mg/I]+Kanamycin [35 mg/I] and incubated at 37° C. at 180 rmp until OD600=0.5, at which point 50 μl of 0.5M IPTG (final 0.1 mM) was added to the culture and incubated at 18° C. for 18h at 120 rpm. On the next day, the produced culture was centrifuged for 15 minutes at 6,000 rpm to harvest the cells, and the pellet was resuspended in 10 ml ice-cold Lysis buffer I (NaCl 500 mM, Tris 20 mM and imidazole 10 mM, pH 8+1 tablet/10 ml of complete protease inhibitor). The resuspended pellet was sonicated (amplitude 30%, on-cycle 1 second, off-cycle 2 seconds repeating for 15 minutes), and the cell lysate was centrifuged for 45 minutes at 30,000 rpm. Following centrifugation, the supernatant was passed through a 0.22 μm filter to generate a cell-free extract.

A gravimetric column was packed with 500 μl of Ni-NTA slurry, and the packing solution was eluted. Three column volumes of Lysis buffer I was passed through the column for equilibration of the resin. The cell-free lysate was passed through the column, collecting the flow-through for later SDS-page analysis. The column was washed with 4 column volumes of Wash buffer II (NaCl 500 mM, Tris 20 mM and imidazole 20 mM, pH 8), collecting fractions for SDS-page analysis. After washing, 5 column volumes of Elution buffer III (NaCl 500 mM, Tris 20 mM and imidazole 250 mM, pH 8) was applied to the column to release the bound protein, collecting the elution fractions for later SDS-PAGE analysis.

The eluted fractions were pooled together, and concentration measured using a NanoDrop (Mw: 145.66 kDa Extinction coefficient (ε molar(M-1 cm-1))=169270) and diluted to a final 1 μM stock solution in SEC buffer (KCl 500 mM, HEPES 20 mM DTT 1 mM).

His-tagged proteins were purified on nickel columns using standard protein purification protocols. The purified Cas12a proteins were incubated with guide RNA and plasmids comprising a target site for said guide RNA. Target plasmids (and control plasmids lacking the target site) were then loaded on a gel to analyze the presence of nicked, linear (cleaved double strand) or supercoiled (neither nicked nor cleaved double strand) plasmid. A reaction was set up in 1× Nuclease buffer (HEPES [20 mM], NaCl [100 mM], MgCl2 [5 mM], EDTA [0.1 mM]) containing the purified LbCas12a variant [100 mM] together with a synthetic guide RNA [200 nM] and a negatively supercoiled pUC19 plasmid substrate [150 fmol] which has in its sequence a target protospacer that perfectly matches the provided guide RNA. First, the LbCas12a variant was incubated in the 1× Nuclease buffer with the guide RNA for 20 minutes at room temperature. After assembling the RNP, the plasmid DNA substrate was added to the reaction and incubated for 1 hour at 37° C. After incubation, the reaction was stopped by adding NEB Purple loading dye, and the reaction was loaded in a 1% agarose gel.

As controls for the plasmid topology, a negative control was produced using the DNA substrate in 1× Nuclease buffer. The linear topology control was produced by digesting the DNA substrate with EcoRI-HF restriction enzyme, and the nicked topology was reproduced using Nb.BbvCI nickase restriction enzymes. All controls were generated using the same input amount of DNA substrate as in the reactions containing the LbCas12a variants.

Surprisingly pRV26004 (SEQ ID NO: 16), which showed a GFP signal comparable to dead Cas12a in the in vivo analysis (suggesting a nickase activity but no or little nuclease activity), showed nicking and cleavage of the target DNA in vitro, at least under the chosen conditions (see FIG. 6B). However, the lid deletion mutant (SEQ ID NO: 15) showed very strong nicking activity with little residual nuclease activity. To determine which strand the lid deletion mutant is cleaving, the nicked DNA fragment was extracted from the gel and analyzed by Sanger run-off sequencing (see FIG. 6C). Three replicates of reverse primer (NTS as template) sequencing of the RuvC^{L del1*} digested target showed termination of the sequencing reaction within the target site, as depicted in the right sequencing chromatogram sketch in FIG. 6C, whereas three replicates of forward primer (TS as template) sequencing showed a continuous sequencing reactions over the target area, as depicted in the left sequencing chromatogram sketch in FIG. 6C. Negative controls showed continuous sequencing reactions for both strands and positive controls with restriction enzymes cleaving either TS or NTS showed termination of the sequencing reaction for the respective strand. The obtained results clearly showed that the lid deletion mutant generates a nick in the displaced, non-target strand, indicating that it acts as a non-target strand nickase.

To improve the RuvC lid deletion mutant further, the cysteine residue at position 931 (Cys/C-931) was substituted by selected alternative residues comprising either a bulky (Trp/W), positively charged (Lys/K), or negatively charged (Glu/E) amino acid. The resulting LbCas12a variants were cloned in a pET (pML-1B, KanR. Addgene #29653) vector, including a 6×Histidine tag at the N terminus of the protein, and expressed in E. coli Rosetta DE3 competent cells as described above. For initial testing of activity, a fluorescent nickase assay was designed (see FIG. 6D). In this assay, a 331 bp PCR substrate in which the target strand (complementary to the used crRNA) is labelled with Cy5, and the non-target strand with Cy3, is incubated with the respective nickase candidates and separated via denaturing gel electrophoresis.

Nicking reactions were performed as described above for the plasmid nickase assay, except that the dual Cy3/Cy5-labeled dsDNA substrates were used. After incubation, the reactions were stopped by digesting samples with Proteinase K for 10 min. Next, TBE-urea sample buffer was added, and samples were heated at 95° C. for 5 to 10 minutes to denature the substrate strands. Samples were separated on a denaturing 10-15% TBE-urea gel at 8-15 mA and imaged for fluorescence in an Amersham Typhoon imaging system.

Fluorescently-labeled DNA substrates in 1× Nuclease buffer were used as non-digested control, while nuclease and nickase controls were generated by incubating the DNA substrate with EcoRI-HF and Nb.BbvCI restriction enzymes, respectively. All controls were generated using the same input amount of labeled DNA substrate as in the reactions containing the LbCas12a variants. As shown in FIG. 6E, reactions with the C931 E (SEQ ID NO: 56) variant showed a clear band at the expected location for cleavage of the non-target strand, indicating that it preferentially nicks the non-target strand. Interestingly, time-series analyses revealed that this mutant also has substantially reduced nuclease activity relative to the original RuvC deletion mutant (RuvC^{L-del1}, SEQ ID NO: 15), with only minor levels of target strand cleavage being detected from 150 min onwards. In contrast, the C931W variant showed stronger nicking specificity and a reduced background of double stranded breaks, but no increase in overall activity, while the C931K variant resulted in increased initial nicking activity, but comparable levels of double-stranded breaks (data not shown). Together these findings show that the C931 E variant is a superior nickase variant exhibiting the highest ratio of nickase versus double-strand break activity among all LbCas12a mutants tested.

### Example 5: Analysis in an In Vitro Transcription-Translation System

In addition to the in vivo GFP/RFP detection method, a second analysis approach was used based on an in vitro cleavage system. Genes encoding a Cas12a variant, a guide RNA and GFP are expressed together in one reaction compartment (one well of a 96-well plate) using a cell-free transcription-translation (TXTL) system (Marshall et al., Mol Cell, 2018). In this assay, the expressed guide RNA targets the GFP-encoding sequence, while GFP fluorescence is measured in each reaction compartment over time using a plate reader. Control reactions are set up with guide RNA that does not target the GFP-encoding sequence. While the GFP fluorescence increases over time in the non-targeting control reactions, Cas-mediated cleavage strongly represses GFP fluorescence.

A particular objective for using Cas12a nickases are paired nickase strategies in which at least two guide RNAs are designed to allow a concerted action of at least two Cas enzymes, which may be the same or may be different Cas enzymes, having nickase activity so that the at least two Cas enzymes having nickase activity introduce at least two individual nicks at the at least one target site and the at least two individual nicks may result in an DSB.

Therefore, the TXTL system has been modified to function as an in vitro double nicking assay. In this assay, the GFP coding sequence is targeted not by one guide RNA but instead by a pair of guide RNAs to create a DSB through the introduction of two nicks.

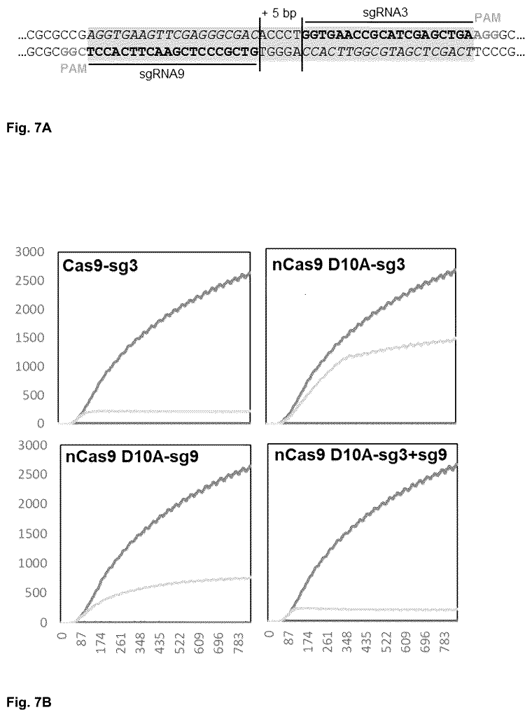

First, the system was set up and optimized using wild type Cas9 and wild type LbCas12a to achieve suitable conditions for high GFP expression and fluorescence detection in non-targeting control samples as well as efficient cleavage by the Cas enzyme in the targeting samples. Next, the double nicking assay was tested and optimized using Cas9 D10A and different pairs of guide RNAs. For illustrative purposes, FIG. 7B shows example results of the in vitro double nicking assay using Cas9 D10A and a pair of guide RNAs (see FIG. 7A). Experiments with Cas12a nickase have started and are ongoing. One aim of this assay is to further test the ability of a Cas12a nickase to introduce a DSB via paired nicks. Experiments are performed with either one Cas12a variant and two suitable, paired guide RNAs or with one Cas12a variant in combination with Cas9D10A and two guide RNAs, suitable for Cas12a targeting and Cas9 targeting, respectively. Apart from the ability to introduce paired nicks and, hence, quantify nickase activity, this in vitro assay can further be used as an additional means to analyze Cas12a variants for residual nuclease activity; thus providing a rapid and scalable tool for quantitative and time-resolved characterization of Cas12 activity.

### Example 6: Analysis of Cas12a Nickase Variants in Bacillus subtilis

The Cas12a variants will be extensively tested in Bacillus subtilis and initial work on these experiments has been conducted. The verification of different Cas12a variants in Bacillus subtilis is set out according to the following protocol:

The Cas9 gene of plasmid pCC0027 (WO2021175759) is replaced by the coding sequence of a Cas12a nickase variant gene by Gibson assembly (NEBuilder® HiFi DNA Assembly Cloning Kit, New England Biolabs) resulting in plasmid pNCP001.

The Cas12a-nickase-based gene deletion plasmid pNCP002 for deletion of the amyB gene of Bacillus subtilis is constructed as described in the following.

The fragment comprising the amyB specific FnCas12a crRNA and the 5′ and 3′ homology regions of the amyB gene (amyB-HomAB) is PCR amplified from plasmid pcrA3 (Wu Y, Liu Y, Lv X, Li J, Du G, Liu L. CAMERS-B: CRISPR/Cpf1 assisted multiple-genes editing and regulation system for Bacillus subtilis. Biotechnol Bioeng. 2020 June; 117(6):1817-1825. doi: 10.1002/bit.27322. Epub 2020 Mar 16. PMID: 32129468.) with primers with flanking Bsal restriction sites. The Cas12a-nickase-based gene deletion plasmid for the amyB gene is subsequently constructed by type-II-assembly with restriction endonuclease Bsal as described (Radeck et al., 2017) with plasmid p00027 and the PCR amplified crRNA-amyB-HomAB region. The reaction mixture is transformed into E. coli DH10B cells (Life technologies). Transformants are spread and incubated overnight at 37° C. on LB-agar plates containing 20 μg/ml Kanamycin. Plasmid DNA is isolated from individual clones and analyzed for correctness by restriction digest and sequencing. The resulting amyE gene deletion plasmid is named pNCP002.

Electrocompetent Bacillus subtilis ATCC6051a cells are prepared as described by Brigidi et al (Brigidi, P., Mateuzzi, D. (1991). Biotechnol. Techniques 5, 5) with the following modification: upon transformation of DNA, cells are recovered in 1 ml LBSPG buffer and incubated for 60 min at 37° C. (Vehmaanperä J., 1989, FEMS Microbio. Lett., 61: 165-170) following plating on selective LB-agar plates.

Electrocompetent Bacillus subtilis ATCC6051a cells are transformed with 1 μg of the amyE deletion plasmid pNCP002 isolated from E. coli DH10B cells following plating on LB-agar plates containing 20 μg/ml kanamycin and incubation overnight at 37° C.

The next day, 20 clones of each transformation reaction are subjected to colony-PCR—to analyze for successful Cas12a-nickase-based deletion of the amyE gene with oligonucleotides located 5′ and 3′ of the homology regions—and further transferred onto fresh LB-agar plates without antibiotics following incubation at 48° C. overnight for plasmid curing.

Correct clones with deleted amyE gene and cured of plasmid pNCP002 are identified and the corresponding B. subtilis ATCC6051a strain with deleted amyE gene isolated.

Likewise, a gene integration is performed into the amyE locus of B. subtilis ATCC6051a. A protein expression construct comprising the GFP-gene under control of the aprE gene promoter is placed in between the 5′ and 3′ homology regions of the amyE gene as described for the Cas9-based construct p00043 (WO2021175759) using Gibson assembly. The resulting Cas12a-nickase-based gene integration plasmid pNCP003 is transformed into electrocompetent Bacillus subtilis ATCC6051a cells and the gene integration procedure is performed as described for the gene deletion procedure.

The resulting B. subtilis ATCC6051a strain with an integrated PaprE-GFP expression cassette in the amyE locus is isolated.

### Example 7: Evaluation of DNA Nicking Activity in Plant Cells

### Cloning Methods and Plasmid Construction

Unless indicated otherwise, cloning procedures carried out for the purpose of the current invention including restriction digest, agarose gel electrophoresis, purification and ligation of nucleic acids, transformation, selection and cultivation of bacterial cells are performed as described (Sambrook J, Fritsch E F and Maniatis T (1989). Sequence analysis of recombinant DNA was performed by LGC Genomics (Berlin, Germany) using the Sanger technology (Sanger et al., 1977). Restriction endonucleases and Gibson Assembly reagents used to construct plasmids are from New England Biolabs (Ipswich, MA, USA). Oligonucleotides are synthesized by Integrated DNA Technologies (Coralville, IA, USA). Codon-optimized genes are from Genewiz (South Plainfield, NJ, USA).

Selected LbCas12a nickase candidates were optimized for expression in plant cells using GeneOptimzer, a BASF proprietary software tool. Different settings were tested with parameters set for codon usage for wheat high-expressing genes and optional removal of major cryptic splice sites. Alternatively, more stringent parameters were used for codon usage with only the most abundant wheat amino acid codons selected during optimization, followed by manual removal of major cryptic splice sites.

Codon-optimized nickase variants were tagged with a SV40 nuclear localization signal at the N-terminus (SEQ ID NO: 36) and a Xenopus-derived Nucleoplasmin C nuclear localization signal at the C-terminus (SEQ ID NO: 37) and synthesized. The synthesized genes were digested with Ncol and Nhel and cloned into a proprietary expression plasmid between the Ncol and Nhel sites. The resulting expression vectors include the maize polyubiquitin (Ubi) promoter (Seq ID NO: 38) for constitutive expression located upstream of the Cas9 gene and a fragment of the 3′ untranslated region of either the nopaline synthase gene of Agrobacterium tumefaciens (SEQ ID NO: 39) or the 35S gene of Cauliflower mosaic virus (SEQ ID NO: 40) at the 3′end.

Guide RNA expression cassettes containing a Cas12a guide RNA composed of a 21-bp direct repeat sequence (SEQ ID NO: 41), a 23-bp protospacer site, and the rice polymerase III terminator sequence (nnnnntttttttt with n being a, c, g, or t) were ordered as synthetic fragments. Expression of the guide RNAs is driven by the polymerase III-type promoter of the rice U6 snRNA gene (SEQ ID NO: 43). The synthesized cassettes were cloned into a standard E. coli vector (pUC derivative) via EcoRV blunt end ligation.

All plasmids were transformed in E. coli for propagation and isolated using a ZymoPure II Plasmid Gigaprep kit for DNA purification (Zymo Research, Irvine, CA, USA).

### Rice Protoplast Isolation and Transfection

Transformation of rice protoplast cells was performed as described by Wang et al. (2014) with minor modifications. Protoplasts were isolated from the sheaths of 3-week-old aseptically grown rice seedlings. Healthy stems and sheaths were bundled in stacks of 20 and cut into fine strips with a sharp razor blade. The strips were then infiltrated with cell wall-dissolving enzyme solution (1.5% cellulase R10 and 0.75% macerozyme R10 in 10 mM KCl and 0.6 M mannitol, pH 7.5) and incubated overnight in the dark with gentle shaking (40 rpm) at 24° C. After enzymatic digestion, the released protoplasts were collected by filtering the mixture through 40-μm nylon meshes and resuspended in W5 solution. The resuspended protoplasts were washed with W5 solution, after which the cell pellet was suspended in MMG solution at a density of 2.5 million cells/ml. For transformation, 200 μl of cells (5×10⁵ cells) were mixed with 20 μg plasmid DNA and 220 μl of freshly prepared polyethylene glycol (PEG) solution. The mixture was incubated for 15-20 min in the dark. After removing the PEG solution, the protoplasts were resuspended in 2 ml of WI solution, transferred into six-well plates, and incubated at 24° C. for at least 48h. Finally, protoplasts were collected by centrifugation at 12,000 rpm for 1 min at room temperature and the pelleted fraction was stored at minus 80° C. until further analysis.

### Oilseed Rape Protoplast Isolation and Transfection

Oilseed rape protoplasts were isolated from the leaves of 4- to 7-week-old aseptically grown plants and transfected as described for rice cells. After enzymatic digestion, the released protoplasts were collected by filtering the mixture through 40-μm nylon meshes and resuspended in W5 solution. The resuspended protoplasts were kept on ice for at least 30 min and allowed to settle by gravity, after which the cell pellet was resuspended in MMG. For transformation, 200 μl of cells (2.5×10⁵) were mixed with 20 μg plasmid DNA and 220 μl of freshly prepared polyethylene glycol (PEG) solution. The mixture was incubated for 15-20 min in the dark. After removing the PEG solution, the protoplasts were resuspended in 2 ml of W5 solution, transferred into six-well plates, and incubated at 24° C. In planta nickase activity assays

A convenient in vitro assay for nickase variants of LbCas12a is to monitor processing of negatively supercoiled dsDNA plasmid substrates isolated from E. coli. Exposing the plasmids to Cas12a-derived nuclease variants allows for discriminating variants that generate DSBs or nicks, by analysis of linear and nicked cleavage products using agarose gel electrophoresis. However, this simple assay cannot easily be performed in planta as the presence of relaxed circles among extracted DNAs is insufficient to infer whether nicking has occurred in vivo, or whether nicking occurred during extraction and/or analysis of DNA. Therefore, different assays were designed to evaluate the performance of the selected Cas12a nickase candidates in plant cells.

A first assay takes advantage of new molecular insights into the pathways and factors that regulate repair of nicks in genomic DNA. As the simplest and most frequent form of DNA damage, nicks are typically repaired either seamlessly or through high-fidelity homology-directed repair. Recent findings, however, have highlighted the potential for nicked genomic DNA to undergo mutagenic repair, including the introduction of single nucleotide variations (Zhang Y, et al. PLoS Genet. 2021 doi: 10.1371/journal.pgen.1009329). Hence, low-level frequency of base substitutions at or near the nick site may be used as a proxy for nickase activity in vivo. In this context, selected nickase variants were co-transfected along with a Cas12a guide RNA (SEQ ID NO: 44) targeting the AAT gene (LOC_Os01g55540.1) in rice protoplasts using PEG-mediated transformation as described above. All Cas12a variants were codon-optimized for monocot plants and transcribed from a maize Ubi promoter. Three days post transfection, protoplasts were harvested by centrifugation and genomic DNA was extracted using the Qiagen DNeasy Plant kit. The AAT target region was amplified by PCR using primers SEQ ID NO: 45 and SEQ ID NO: 46 and subjected to amplicon deep sequencing.

As shown in FIG. 8A, transfection of WT LbCas12a (SEQ ID NO:1) resulted in high frequencies of indels at the predicted cut site (average 22.54%), demonstrating efficient generation of double-strand breaks (DSBs). On-target indels were also frequently observed with the R1138A and K932G/N933G mutants (mutations relate to reference sequence SEQ ID NO: 1). Both variants showed indels in 2.98% and 0.62% of total sequencing reads, respectively, which corresponds to 13.21% and 2.73%, respectively, of the indel-inducing activity observed with the Cas12a nuclease control (FIG. 8A). Interestingly, the K932G/N933G/S934A/R935G quadruple mutant (SEQ ID NO 14) induced much fewer indels (average 0.18%, i.e. less than 1% relative to WT Cas12a). Also, the K932G/N933G/S934A/R935G quadruple variant supported a higher number of base substitutions at the AAT target site (up to 1.09% of total sequencing reads) compared to both R1138A and K32G/N933G variants (FIG. 8B). Comparing the number of NGS reads with indels versus those with base conversions further highlighted the differences between the various mutants (FIG. 8C). Unlike Cas12a-R1138A and Cas12a-K932G/N933G, which produced levels of indels reaching 99.44% and 49.04%, respectively, Cas12a-K932G/N933G/S934A/R935G generated predominantly base changes (86.19% of edited sequence reads). Although nicked DNA can in rare cases be processed via a DSB intermediate and result in a NHEJ event (Certo et al., 2011 doi: 10.1038/nmeth.1648), the high ratio of indels versus base changes observed for both R1138A and K932G/N933G suggests substantial nuclease activity for the latter variants.

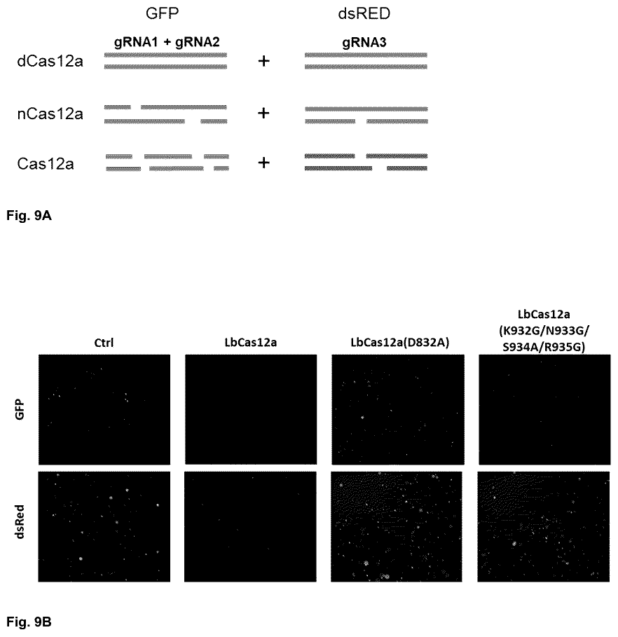

To further assess in planta nickase activity, a dual-plasmid reporter system was devised akin to the GFP/RFP system used in E. coli (example 3). In this system, a plasmid encoding an engineered GFP reporter (SEQ ID NO: 47) harboring two Cas12a targeted sites located in close proximity on opposite strands within the GFP-coding sequence and a plasmid encoding an engineered dsRed reporter (SEQ ID NO: 48) carrying a single Cas12a target site are co-transfected into rice protoplast cells along with the selected Cas12a nickase variant and three Cas12a gRNAs targeting the GFP (SEQ ID NO: 49/SEQ ID NO: 50) and dsRed (SEQ ID NO: 51) reporters, respectively (see FIG. 9A). Three days post transfection, the fluorescent signature of transfected cells can be used to discriminate nickase from catalytically active and inactive enzymes as cells transfected with dead Cas12a will show both GFP and dsRed; cells expressing WT Cas12a will yield no or minimal GFP and dsRed; and cells expressing a nickase will be positive for dsRed (due to single nicking) but low in GFP (due to double nicking).

FIG. 9B shows the results for protoplasts transfected with plasmids encoding either WT LBCas12a (SEQ ID NO:1), catalytically inactive Cas12a-D832R (mutation relates to reference sequence SEQ ID NO: 1), or the Cas12a-K932G/N933G/S934A/R935G (SEQ ID NO:14) variant. Expression of WT Cas12a resulted in a strong reduction in the number of GFP- and RFP-positive cells relative to that in cells transfected with the fluorescent reporters only. In contrast, GFP and dsRed fluorescence with the dead Cas12a variant was equivalent to that in the positive controls, while transfecting cells with the Cas12a quadruple variant resulted in a reduction of GFP signal but not dsRed.

In a third activity assay, base-editing outcomes induced by LbCas12a nickase variants were compared with those by WT LbCas12a. In the absence of a suitable variant that nicks the non-edited strand, Cas12a base editors routinely use catalytically inactive Cas12a as the Cas moiety. By analogy with previously characterized Cas9 base editors (Komor et al., 2016; Nishida et al., 2016; Gaudelli et al., 2017), it is reasonable to assume that use of Cas12a nickases will influence base editing activity. That is, variants that nick the non-edited strand (i.e., target strand) are expected to increase editing levels, while nickase variants that target the edited strand should lower editing efficiencies.

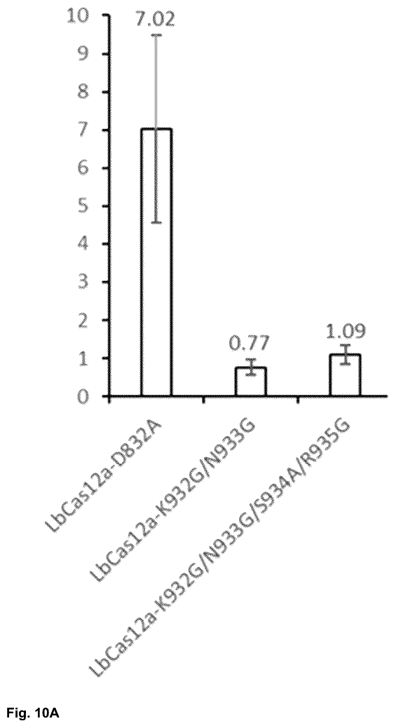

Exploiting this phenomenon, different nickase candidates were introduced into a LbCas12-BE (LbCas12 base editing) construct and editing at the AAT target site was measured after three days by amplicon deep sequencing. As shown in FIG. 10A, Cas12a-mediated base editing by K932G/N933G (mutations relates to reference sequence SEQ ID NO: 1) and K932G/N933G/S934A/R935G (SEQ ID NO: 14) was reduced by approximately 9-fold and 7-fold, respectively, compared to the corresponding D832A (mutation relates to reference sequence SEQ ID NO: 1) variant. Importantly, as shown in FIG. 10B, BE-K932G/N933G also yielded high levels of indel formation (average 10.81%), suggesting that the induction of DSBs and subsequent NHEJ repair, rather than DNA nicking, contributes to the decrease in editing. BE-K932G/N933G/S934A/R935G induced indels at much lower frequencies (<1%) than BE-K932G/N933G, showing an almost 10-fold reduction in the percentage of reads with indels. The difference in editing outcomes between the Cas12a double and quadruple variants was also evident from aligning the 20 most abundant sequencing reads (data not shown). Whereas the quadruple mutant-derived base editor edited different bases in a window of C5 to C22 (counting the end distal to the protospacer-adjacent motif as position 1), introduction of K932G/N933G almost invariably resulted in deletions with very few accompanying base edits. Together with the relatively high frequency of base changes and low-level indel formation at individual nick sites, as well as the reduction of GFP− but not dsRed-derived fluorescence in the dual-color reporter assay, these findings strongly suggest that the LbCas12a-K932G/N933G/S934A/R935G quadruple variant exhibits significant nickase activity in plant cells with no or at least very low residual nuclease activity.

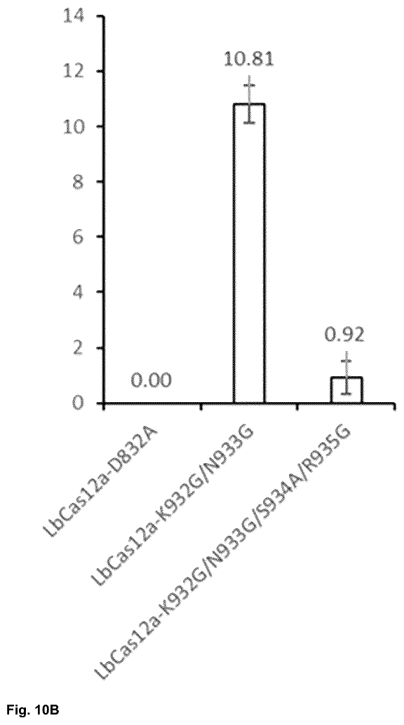

The different activity assays were also used to assess the in planta performance of the RuvC lid deletion mutant (RuvC^{L-del1}, SEQ ID NO: 15) and its C931E variant (SEQ ID NO: 56). As shown in FIG. 11, transfection of the RuvC lid deletion mutant in rice protoplasts along with a Cas12a guide RNA targeting the AAT gene resulted in strongly reduced indel formation as compared to WT LbCas12a while even lower levels of on-target indels were observed with the RuvC lid C931E mutant. Like the LbCas12a-K932G/N933G/S934A/R935G quadruple variant, both mutants also induced detectable levels of base substitutions (up to 90% of edited sequence reads) at the AAT target site, a phenomenon which might be indicative of nickase activity.

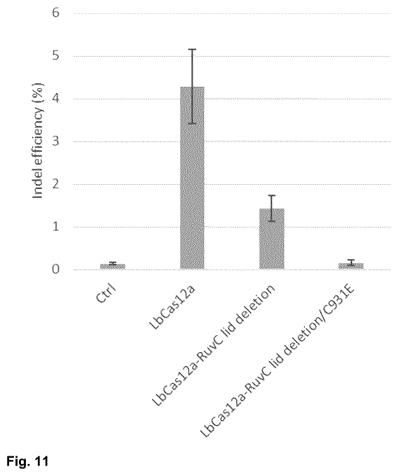

To evaluate nickase activity further, the RuvC lid deletion and C931 mutations were introduced into an LbCas12-BE construct and editing at the AAT target site was quantified after three days by amplicon deep sequencing. The results are shown in FIG. 12. Pooled over six independent experiments, the RuvC lid deletion mutations reduced Cas12a-base editing by almost 4.5-fold compared to the corresponding variant LbCas12a-D832A, while the additional C931 E mutation resulted in a 1.4-fold decrease in editing efficiency (see FIG. 12A). A similar picture emerged when targeting the FAD2 gene (LOC106452409) in oilseed rape (Brassica napus) protoplasts. In this case, transfecting Cas12a base editors harboring the RuvC lid deletion and C931E mutations together with a FAD2-targeting gRNA (SEQ ID NO: 57) lowered base editing by 1.98- and 4.43-fold respectively as compared to the Cas12a D832A BE construct (mutation relates to reference sequence SEQ ID NO: 1) (see FIG. 12B). Considering that the RuvC lid deletion mutant preferentially cuts the non-target strand (see FIG. 6E) and given the low levels of residual nuclease activity of both mutants (see FIG. 11 and FIGS. 6B and 6E), it is reasonable to assume that the observed reduction in base editing is due to nicking of the edited strand.

Finally, the in planta activity of the different variants was evaluated in a dual nickase experiment. In this approach, indel formation at a target site is evaluated using nickase candidates directed by either single guides or pairs of offset guides targeting opposite DNA strands. While single nicks are predominantly repaired via high-fidelity base excision repair, cooperative nicking of opposite DNA strands is expected to generate site-specific double-strand breaks and subsequent formation of indels. As demonstrated previously for Cas9 nickases (Ran et al., DOI: 10.1016/j.cell.2013.08.021), different factors may influence cooperative nicking leading to indel formation, including steric hindrance between two adjacent Cas12a RNPs, overhang type, and sequence context. To assess how Cas12a gRNA target sequences and offsets between the guides might affect the generation of indels, sets of gRNA pairs targeting the rice OsDEP1 gene (LOC106452409) and separated by a range of offset distances from +62 to −95 bp to create either 5′ or 3′ overhangs were designed and tested for their ability to induce on-target indels in rice protoplasts co-transfected with the RuvC lid deletion variant (RuvCL del1, SEQ ID NO: 15; gRNAs: SEQ ID NO: 57 to SEQ ID NO: 73).

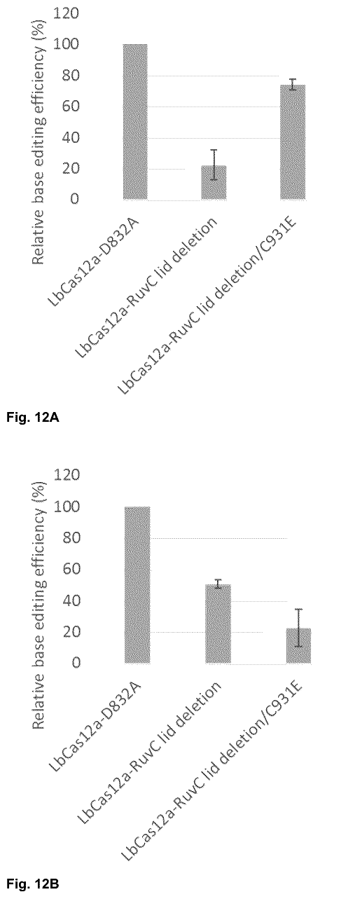

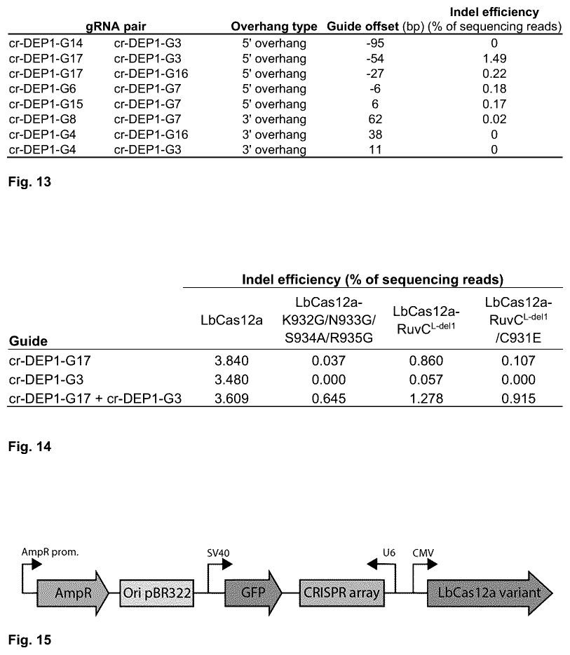

As shown in FIG. 13, only gRNA pairs creating 5′ overhangs with at least 9 bp offset between the guides were able to mediate detectable indel formation. Notably, a considerable fraction of the induced mutations showed large deletions (>50 bp) between the two nick sites, which likely results from cooperative cutting of opposite DNA strands by sequentially or simultaneously bound nickases. The highest indel frequency (up to 1.49% of sequencing reads) was observed for the gRNA3+gRNA17 pair creating a 64-bp 5′ overhang. Using the gRNA3/gRNA17 pair, we next compared the indel frequency induced by paired nickases to that induced by a single nickase or WT LbCas12a. As expected, transfection of WT LbCas12a with gRNA3 or gRNA17 alone led to significant indel formation at the respective target sites (average of 3.84% and 3.48%, respectively), whereas very few indels were detected when using single guides with either the LbCas12a-K932G/N933G/S934A/R935G quadruple variant, the RuvC lid deletion mutant or its C931E variant (see FIG. 14). Obvious differences between WT and the Cas12a nickase candidates were also evident when testing paired gRNAs. Indeed, while the indel frequency induced by WT LbCas12a and co-transfected gRNA3 and gRNA17 was comparable to those generated by WT Cas12a paired with each gRNA alone (3.86% versus 3.84% and 3.48%, respectively), co-delivering both gRNAs and the Cas12a nickase candidates had a synergistic effect and strongly potentiated indel formation compared to single nickases. This was particularly evident for the quadruple and C931 E mutants where no or very few indels were detected for the single nickase, whereas the combination of guides successfully generated on-target indels at frequencies of 0.65 and 0.91%, respectively. In a similar vein, double targeting of the RuvC lid deletion variant by the gRNA3/gRNA17 pair also induced indel formation at frequencies significantly greater than single gRNA targeting. Together these findings not only illustrate the robust performance of the different RuvC lid nickase variants in planta, but also show that these Cas12a mutant proteins can be leveraged to facilitate targeted DNA double-strand breaks using paired guide RNAs.

### Example 8: Gene Modifications in Ashbya gossypii Using Cas12a-Nickases

### Assembly of the CRISPR-Cas12a-Nickase Vector

The Cas12a-nickase system is assembled in a single vector containing all the required modules for genomic editions. The Ashbya gossypii CRISPR-Cas9 vector is used as a backbone that includes the replication origins (yeast 2 μm and bacterial ColE1) and the resistance markers (AmpR and G418R) (Jimenez A, MuAoz-Fernandez G, Ledesma-Amaro R, Buey R M, Revuelta J L. One vector CRISPR-Cas9 genome engineering of the industrial fungus Ashbya gossypii. Microb Biotechnol 2019; 12:1293-1301). The donor DNA and the modules for expression of Cas12a-nickase and crRNAs are assembled as follows: a synthetic codon-optimized ORF of the Cas12a-nickase enzyme (LbCas12a-nickase) with a SV40 nuclear localization signal is assembled with the promoter and terminator sequences of the A. gossypii TSA1 and ENO1 genes, respectively. The expression of the crRNA is driven by the promoter and terminator sequences of the A. gossypii SNR52 gene, which is transcribed by RNA Polymerase III. Synthetic donor DNA comprising the corresponding genomic edition is also assembled in the nCas12a-nickase vector. The assembly of the fragments is achieved following a Golden Gate assembly method as previously described (Ledesma-Amaro R, Jiménez A, Revuelta J L. Pathway grafting for polyunsaturated fatty acids production in A. gossypii through Golden Gate Rapid Assembly. ACS Synth Biol 2018; 7:2340-2347). A directional cloning strategy is used, by introducing Bsal sites at the ends of the fragments. The Bsal sites are flanked by sequences of 4-nucleotide (nt) sticky ends. Hence, after Bsal digestion, all the modules contain compatible 4-nt sticky ends that facilitate a single-step directional assembly of the Cas12a-nickase vector.

Using the described cloning strategy, Cas12a-nickase systems, based on different Cas12a-nickase variants, are designed to inactivate the ADE2 gene in A. gossypii. ADE2-defective mutants show a red color due to accumulation of an intermediate of the purine synthesis pathway. Thereby the ADE2 gene is a suitable reporter for gene inactivation. The same system was already used to show the applicability of the CRISPR-Cas12a system for A. gossypii (Jimenez A, Hoff B, Revuelta J L. Multiplex genome editing in Ashbya gossypii using CRISPR-Cas12a. New Biotechnol 2020; 57:29-33). In this experiment the same crRNA sequences and donor DNA sequence are chosen, the only difference is the use of a Cas12a-nickase to induce a single strand DNA break and with this the DNA repair system in Ashbya.

Transformation of A. gossypii and Cas12a-Nickase-Mediated Genome Editing

5-10 μg of the above-described plasmid encoding one of the Cas12a-nickase variants as well as the ADE2-specific crRNA and donor DNA sequences are used to transform spores of the A. gossypii wild-type strain ATCC10895 as described previously (Jimenez A, Santos M A, Pompejus M, Revuelta J L. Metabolic engineering of the purine pathway for riboflavin production in Ashbya gossypii. Appl Environ Microbiol 2005; 71:5743-5751). Heterokaryotictransformants are selected on G418-containing MA2 medium, thus confirming the uptake of the plasmid. The G418-resistant colonies are isolated and grown up again at 30° C. in G418-MA2 medium for 2 days to facilitate genomic editing events. The loss of the CRISPR-Cas12a-nickase plasmid is carried out after sporulation of the heterokaryotic clones in sporulation media lacking G418. Homokaryotic clones are isolated in MA2 media lacking G418. The desired genomic inactivation of the ADE2 gene leads to red colonies on the agar plate. Genomic DNA of the red transformants is isolated and the transformants are analyzed via PCR and sequencing to confirm desired ADE2 editing.

The sequencing results of the obtained transformants are expected to show that using the Cas12anickase instead of Cas12a nuclease leads to a higher number of clones carrying the desired short ADE2 deletion while fewer clones should carry only a random single point mutation resulting from the non-homologous end-joining repair. Thereby, nuclease and nickase activity can be discriminated by sequencing. In line with studies on Cas9 nickases, it is expected that the efficiency to obtain the specific HDR-mediated genome editing event is improved using the Cas12a-nickase.

### Example 9: In Vivo Double Nicking in Yeast Cells

The ADE2 disruption strategy (cf. example 8) is further used to test for in vivo paired nicking in fungal cell. Selected Cas12a nickase candidates will be tested in vivo for nuclease and nickase activity in yeast cells by targeting the reporter gene ADE2 with either a single guide RNA or, in parallel, with a pair of guide RNAs, similar to the in vivo GFP/RFP (example 3) or GFP/dsRed (example 7) assays. Loss of ADE2 leads to a red phenotype in yeast cells due the accumulation of a red intermediate in the adenine synthesis pathway. Yeast cells will be transformed with different Cas12a nickase candidates and either a single guide RNA or a suitable pair of guide RNAs targeting the ADE2 gene. Nuclease activity of a Cas12a protein should cause a red phenotype with both the single and the pair of guide RNAs, while nickase activity should only cause a red phenotype only when the guide RNA pair is present. A dead Cas12a variant should not cause a red phenotype in either scenario.

### Example 10: Analysis of Cas12 Nickase Variants in Mammalian Cells

Further examples to test selected nCas12a variants, or orthologs thereof, are planned in immortalized cell lines, such as HEK293, HeLA, A549, or Jurkat cells, primary mouse and human cells, embryos, egg cells, stem cells and the like.

Target cells of interest can be transfected with selected nCas12a variants or orthologs thereof as disclosed herein, properly codon-optimized and using cell-compatible NLS sequences and regulatory sequences optimized for a given target cell of interest, and the nCas12 enzymes can be provided together with either one guide RNA (a single crRNA, or a crRNA:.tracrRNA heteroduplex, or a chimeric single guide RNA), or a pair of guide RNAs suitable for a paired nickase approach. Guide RNAs or guide RNA pairs may target any chromosomal target or a target on a plasmid such as a reporter construct for an easier assessment of nickase activity and residual nuclease activity. Transfection and transformation protocols (chemical (nucleofection, lipofection etc.), viral-mediated, physical (e.g., bombardment, electroporation, microinjection for embryos, oocytes or zygotes), biological, using vectors and plasmids), buffers and equipment are known to the skilled person for a given target cell of interest.

To characterize the nicking activity of the LbCas12a-RuvC lid deletion variant in mammalian cells, three different genes are selected (EMX1, DYRK1A and GRIN2BA) that are targeted with different variants of LbCas12a (wild type, nickase and dead; corresponding gRNAs: SEQ ID NO: 74 to SEQ ID NO: 79). In principle, the production of a single nick should not induce indel formation in the target site, contrary to paired nicking, which produces a double strand break (DSB), leading to non-homologous end joining (NHEJ) and subsequent indel formation. LbCas12a nickases are not expected to produce a DSB when only one locus is targeted (one guide) but should lead to DSB generation when two adjacent loci are targeted simultaneously (two guides). In this manner, using paired nicking can provide greater on-target cleavage specificity and yield higher frequencies of accurately edited cells when compared to the standard double-stranded DNA break-dependent approach.

Cloning and replication of the expression vectors is performed in the E. coli DH10b cloning strain. The following modules are integrated In the E. coli plasmid (pBR322, selection marker AmpR under control of native bla/AmpR promoter): (i) genes encoding one of the three LbCas12a variants (wild type (LbCas12a-WT), nickase (e. g. LbCas12a-RucC lid deletion variant) and dead (LbCas12a-dead)) downstream of the CMV promoter, (ii) a synthetic CRISPR array (allowing for targeting one of the 3 target genes) downstream of the U6 promoter, and (iii) a gene encoding a GFP marker downstream of the SV40 promoter (see FIG. 15). Upon individual transfection of each of these plasmids into human cells (HEK293), the Cas12a/CRISPR genes and gfp gene are transiently expressed, and Cas12a/crRNA RNP complexes are formed. Different combinations of the LbCas12a variants and the guides are needed to evaluate paired nicking in the selected loci. To this end, sets of different plasmids are produced (3 nucleases×3 loci×2 CRISPR arrays (single guide array or double guide array)).

HEK293 cells are transfected using lipofectamine following standard procedures and subsequently incubated. Due to variable transfection efficiencies and to avoid sequencing of non-transfected cells, the resulting bacterial culture is FACS sorted to enrich for GFP-positive cells (indication that transfection was successful). After pooling the transfected population, chromosomal DNA is extracted from each population and PCR reactions are performed to generate amplicons of the three target sites followed by amplicon deep sequencing (Illumina) to calculate the frequency of indel formation in each treatment. A detailed protocol is described below.

| | Locus | | | |
|---|---|---|---|---|
| | 5′->3′ | Spacer1 | Spacer2 | |
| Target | (PAM to PAM) | (5′->3′) | (5′->3′) | Gap |
| EMX1 | TTTCACTTGGGTG | ACTTGGGTGC | GATGGCGACT | 15 |
| | CCCTAGGAAGCTG | CCTAGGAAGC | TCAGGCACAG | nt |
| | CCTCTGGCCTATC | (SEQ ID | (SEQ ID | |
| | CTGTGCCTGAAGT | NO: 115) | NO: 116) | |
| | CGCCATCCAAA | | | |
| | (SEQ ID | | | |
| | NO: 114) | | | |
| DYRKIA | TTTAAGGGGGTAG | AGGGGGTAGC | CTTACTTCCC | 12 |
| | CATTTCTCTGTAA | ATTTCTCTGT | CTCCCACACT | nt |
| | ACTCCACAGAAGT | (SEQ ID | (SEQ ID | |
| | GTGGGAGGGGAAG | NO: 118) | NO: 119) | |
| | TAAGTAAA | | | |
| | (SEQ ID | | | |
| | NO: 117) | | | |
| GRIN2BA | TTTAGCGCTGTCA | GCGCTGTCAA | AGGGTCCTCA | 11 |
| | AGAACCAGAATGT | GAACCAGAAT | CTGTTCTATT | nt |
| | CTTAACATTAATA | (SEQ ID | (SEQ ID | |
| | GAACAGTGAGGAC | NO: 121) | NO: 122) | |
| | CCTGAAA | | | |
| | (SEQ ID | | | |
| | NO: 120) | | | |

Table 1 displays an overview of the selected loci and spacers used for paired nicking in HEK293 cells.
The sequences above are provided as SEQ ID NOS: 114 to 122.

Protocol
1. Cloning
   a. Produce the different plasmids with either LbCas12a Lid2.3, LbCas12a dead or LbCas12a WT and the respective guides (single guide array or double guide array) for a total of 18 plasmids.
   b. Golden Gate cloning using Bsal restriction enzyme
2. HEK293 cells transfection
   a. Cells are transfected with the desired plasmid using lipofectamine 2000
   b. Cells are cultured in an incubator at 37° C. for 6 h. After 6 h, the Opti-MEM medium is replaced with D-MEM to optimize cell growth, and the cells are incubated at 37° C. for at least 48 h prior to sorting.
3. GFP+sorting
   a. FACS sorting to pool only GFP+cells
4. DNA extraction and isolation
5. PCR to produce sequencing amplicon
6. NGS sequencing

### Data Analysis

### Example 11: Base Editing and Prime Editing

Selected nickase variants will be tested in in base editing systems, (both single and dual base editors using different set-ups with different cytidine and/or adenosine deaminases and different linker regions) and optionally in prime editing systems (with different reverse transcriptases, different pegRNA design, with and without an additional guide RNA targeting the edited sequence, i.e. PE2 and PE3). Base editing, and optionally prime editing, will be tested in the most important target systems, including crop plants and optionally fungal systems and human cells. Exemplary first results for base editing in rice protoplasts are shown in FIGS. 10A and 10B (Example 7). While these results showed a negative effect on base editing levels of the tested Cas12a nickase variants (due to cutting of the edited strand), it should be noted that these mutants might be adopted to improve editing efficiencies in a manner analogous to the previously described Cas9 PPE3 system (Anzalone et al., 2019). In this approach, selected NTS-nickases are complexed with a nicking gRNA and the resulting RNP is co-delivered with a Cas12a base editor harboring catalytically inactive Cas12a. While the Cas12a base editor is directed to the target site by a first gRNA, the nicking gRNA will direct the NTS nickase to cut the non-edited DNA strand, which should facilitate favorable DNA repair by inducing cells to use the edited strand as a repair template. Optionally, the nicking gRNA can be designed to specifically target the edited sequence, thereby preventing nicking of the non-edited strand until after editing occurs (Anzalone et al., 2019). Since the optimal nicking position may vary depending on the genomic site, a variety of non-edited strand nick locations should be tested using gRNAs that induce nicks positioned 5′ or 3′ and at different distances away from the edit site, e.g. 10 to 120 bp.

### REFERENCES

Anzalone A V, Randolph P B, Davis J R, Sousa A A, Koblan L W, Levy J M, Chen P J, Wilson C, Newby G A, Raguram A, Liu D R. (2019) Search-and-replace genome editing without double-strand breaks or donor DNA.Nature. 2019 December; 576(7785):149-157.
Cong L, Ran F A, Cox D, Lin S, Barretto R, Habib N, Hsu P D, Wu X, Jiang W, Marraffini L A, Zhang F. (2013) Multiplex genome engineering using CRISPR/Cas systems. Science. 2013 Feb. 15; 339(6121):819-23.
Dianov G L, Hubscher U (2013) Mammalian base excision repair: the forgotten archangel. Nucleic Acids Res. 2013 Apr. 1; 41(6):3483-90
Gasiunas G, Barrangou R, Horvath P, Siksnys V. (2012) Cas9-crRNA ribonucleoprotein complex mediates specific DNA cleavage for adaptive immunity in bacteria. Proc Natl Acad Sci USA. 2012 Sep. 25; 109(39):E2579-86
Gaudelli N M, Komor A C, Rees H A, Packer M S, Badran A H, Bryson D I, Liu D R. (2017) Programmable base editing of A•T to G•C in genomic DNA without DNA cleavage. Nature. 2017; 551:464-471.
Jin S, Lin Q, Gao Q, Gao C. Optimized prime editing in monocot plants using PlantPegDesigner and engineered plant prime editors (ePPEs). Nat Protoc. 2022 Nov. 25. doi: 10.1038/s41596-022-00773-9.
Jinek M, Chylinski K, Fonfara I, Hauer M, Doudna J A, Charpentier E. (2012) A programmable dual-RNA-guided DNA endonuclease in adaptive bacterial immunity. Science. 2012 Aug. 17; 337(6096):816-21.
Karvalis et al. Nucleic Acids Res. (2020); 48(9):5016-5023. doi: 10.1093/nar/gkaa208
Kim et al. Nat Biotechnol. (2022); 40(1):94-102; doi: 10.1038/s41587-021-01009-z
Komor A C, Kim Y B, Packer M S, Zuris J A, Liu D R. (2016) Programmable editing of a target base in genomic DNA without double-stranded DNA cleavage. Nature. 2016; 533:420-424
Kosicki M, Tomberg K, Bradley A. (2018) Repair of double-strand breaks induced by CRISPR-Cas9 leads to large deletions and complex rearrangements. Nat Biotechnol. 2018; 36:765-77
Kuscu C, Arslan S, Singh R, Thorpe J, Adli M. (2014) Genome-wide analysis reveals characteristics of off-target sites bound by the Cas9 endonuclease. Nat Biotechnol. 2014 July; 32(7):677-83
Lin Q, Zong Y, Xue C, Wang S, Jin S, Zhu Z, Wang Y, Anzalone A V, Raguram A, Doman J L, Liu D R, Gao C. Prime genome editing in rice and wheat. Nat Biotechnol. 2020 May; 38(5):582-585. doi: 10.1038/s41587-020-0455-x. Epub 2020 Mar 16. PMID: 32393904.
Mali P, Yang L, Esvelt K M, Aach J, Guell M, DiCarlo J E, Norville J E, Church G M. (2013) RNA-guided human genome engineering via Cas9. Science. 2013 Feb. 15; 339(6121):823-6
Marzec M, Braszewska-Zalewska A, Hensel G. Prime Editing: A New Way for Genome Editing. Trends Cell Biol. 2020 April; 30(4):257-259. doi: 10.1016/j.tcb.2020.01.004. Epub 2020 Jan. 27. PMID: 32001098.
Nishimasu H, Nureki O, Structures and mechanisms of CRISPR RNA-guided effector nucleases, Current Opinion in Structural Biology, Volume 43, 2017, pages 68-78, ISSN 0959-440X, https://doi.org/10.1016/j.sbi.2016.11.013
Needleman S B, Wunsch C D. A general method applicable to the search for similarities in the amino acid sequence of two proteins. J Mol Biol. 1970 March; 48(3):443-453. doi: 10.1016/0022-2836(70)90057-4. PMID: 5420325.
Ran F A, Hsu P D, Lin C Y, Gootenberg J S, Konermann S, Trevino A E, Scott D A, Inoue A, Matoba S, Zhang Y, Zhang F. (2013) Double nicking by RNA-guided CRISPR Cas9 for enhanced genome editing specificity. Cell. 2013 Sep. 12; 154(6):1380-9.
Shin H Y, Wang C, Lee H K, Yoo K H, Zeng X, Kuhns T, Yang C M, Mohr T, Liu C, Hennighausen L. (2017) CRISPR/Cas9 targeting events cause complex deletions and insertions at 17 sites in the mouse genome. Nature Comm. 2017; 8:15464
McConnell Smith A, Takeuchia R, Pellenz S, Davis L, Maizels N, Monnat R J, Stoddard B L. (2009) Generation of a nicking enzyme that stimulates site-specific gene conversion from the I-Anil LAGLIDADG homing endonuclease. Proc Natl Acad Sci USA. 2009; 106(13):5099-5104.
Selkova et al. RNA Biol. (2020); 17(10):1472-1479; doi: 10.1080/15476286.2020.1777378
Shaner N C, Steinbach P A, Tsien R Y. A guide to choosing fluorescent proteins. Nat Methods. 2005 December; 2(12):905-9. doi: 10.1038/nmeth819. PMID: 16299475.
Sretenovic S, Qi Y. Plant prime editing goes prime. Nat Plants. 2022 January; 8(1):20-22. doi: 10.1038/s41477-021-01047-0.
Stella S. et al., Conformational Activation Promotes CRISPR-Cas12a Catalysis and Resetting of the Endonuclease Activity, Cell, 2018, vol. 175(7), https://doi.org/10.1016/j.cell.2018.10.045
Swarts, D. C., Van der Oost, J., Jinek, M. (2017) Structural Basis for Guide RNA Processing and Seed-Dependent DNA Targeting by CRISPR-Cas12a. Molecular Cell. 66, 221-233
Swarts D C, Jinek M. (2019). Mechanistic Insights into the cis- and trans-Acting DNase Activities of Cas12a. Molecular Cell. 2019; 73:589-600
Tan S, Evans R R, Dahmer M L, Singh B K, Shaner D L. Imidazolinone-tolerant crops: history, current status and future. Pest Manag Sci. 2005 March; 61(3):246-57. doi: 10.1002/ps.993. PMID: 15627242.
Tsai S Q, Zheng Z, Nguyen N T, Liebers M, Topkar V V, Thapar V, Wyvekens N, Khayter C, Lafrate A J, Le L P, Arayee M J, Joung J K. (2015) GUIDE-seq enables genome-wide profiling of off-target cleavage by CRISPR-Cas nucleases. Nat. Biotechnol. 2015; 33:187-197.
Weusthuis, R. A., Mars, A. E., Springer, J., Wolbert, E. J., Van der Wal, H., De Vrije, T. G., Levisson, M., Leprince, A., Houweling-Tan, G. B., Pha Moers, A., Hendriks, S. N., Mendes, O., Griekspoor, Y., Werten, M. W., Schaap, P. J., Van der Oost, J., Eggink, G. (2017) Monascus ruber as cell factory for lactic acid production at low pH. Metabolic Engineering 42, 66-73
Xu R, Li J, Liu X, Shan T, Qin R, Wei P. Development of Plant Prime-Editing Systems for Precise Genome Editing. Plant Commun. 2020 Apr. 8; 1(3):100043. doi: 10.1016/j.xplc.2020.100043. PMID: 33367239; PMCID: PMC7747961.
Yamano T, Nishimasu H, Zetsche B, Hirano H, Slaymaker I M, Li Y, Fedorova I, Nakane T, Makarova K S, Koonin E V, Ishitani R, Zhang F, Nureki O. Crystal Structure of Cpf1 in Complex with Guide RNA and Target DNA. Cell. 2016 May 5; 165(4):949-62. doi: 10.1016/j.cell.2016.04.003. Epub 2016 Apr. 21. PMID: 27114038; PMCID: PMC4899970.
Yamano T, Zetsche B, Ishitani R, Zhang F, Nishimasu H, Nureki O. Structural Basis for the Canonical and Non-canonical PAM Recognition by CRISPR-Cpf1. Mol Cell. 2017 Aug. 17; 67(4):633-645.e3. doi: 10.1016/j.molcel.2017.06.035. Epub 2017 Aug. 3. PMID: 28781234; PMCID: PMC5957536.
Zetsche B, Gootenberg J S, Abudayyeh 00, Slaymaker I M, Makarova K S, Essletzbichler P, Volz S E, Joung J, van der Oost J, Regev A, Koonin E V, Zhang F. Cpf1 is a single RNA-guided endonuclease of a class 2 CRISPR-Cas system. Cell. 2015 Oct. 22; 163(3):759-71. doi: 10.1016/j.cell.2015.09.038. Epub 2015 Sep. 25. PMID: 26422227; PMCID: PMC4638220.
Zhang L, Jia R, Palange N J, Satheka A C, Togo J, An Y, Humphrey M, Ban L, Ji Y, Jin H, Feng X, Zheng Y. (2015) Large genomic fragment deletions and insertions in mouse using CRISPR/Cas9. PLoS ONE. 2015; 10, e0120396
Zhang et al., Mechanisms for target recognition and cleavage by the Cas12i RNA-guided endonuclease, Nat Struct Mol Biol, (2020), 27(11): 1069-1076, doi: 10.1038/s41594-020-0499-0
Zhang B., et al., Mechanistic insights into the R-loop formation and cleavage in CRISPR-Cas12i1, Nature Communications, 12:3476, (2021) https://doi.org/10.1038/s41467-021-23876-5.

1. An engineered Cas12a enzyme having nickase activity (nCas12a), or a catalytically active fragment thereof, the engineered Cas12a enzyme comprising at least one mutation in its core lid domain, wherein the mutation in the core lid domain is selected from:
(i) at least three point mutations of three consecutive positions within the core lid domain; or
(ii) a deletion of at least two consecutive positions within the core lid domain; or
(iii) a combination of at least one first point mutation at least one position within the core lid domain and
(iiia) at least one deletion of at least one position within the core lid domain, and/or
(iiib) at least one, preferably at least two, at least three, or at least four further point mutation(s) at a different position in comparison to the first point mutation within the core lid domain, wherein the position(s) of the further point mutation(s) is/are not in consecutive order with the position(s) of the at least one first point mutation;
(iv) one point mutation at a position within the core lid domain;
wherein the at least one mutation in the core lid domain confers broad spectrum nickase activity, wherein the core lid domain reference sequence comprises a sequence as defined in SEQ ID NO: 13, optionally a complex additionally comprising at least one compatible guide RNA, or a sequence encoding the same, forming a complex with the cognate engineered Cas12a enzyme having nickase activity, or the catalytically active fragment thereof.

2. The engineered Cas12a enzyme or the catalytically active fragment thereof of claim 1,
wherein the engineered Cas12a enzyme is based on a wild-type Cas12a sequence according to any one of SEQ ID NOs: 1 to 12, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the corresponding wild-type sequence as reference sequence, or an ortholog or homolog of a sequence according to any one of SEQ ID NOs: 1 to 12 having at least 95%, 96%, 97%, 98% or at least 99% sequence identity to the corresponding ortholog or homolog sequence as reference sequence,
wherein the at least three point mutations in three consecutive amino acids are positioned within positions 2 to 16 with reference to SEQ ID NO: 13, and/or wherein the deletion is a deletion of at least two, at least three, at least four, at least five, at least six at least seven, at least eight, at least nine, at least ten, at least eleven, at least twelve, at least thirteen, at least fourteen, at least fifteen, at least sixteen, or at least seventeen consecutive positions within the core lid domain,
wherein the mutation is a deletion of at least four, at least five, at least six at least seven, or at least all eight positions 6 to 13 with reference to SEQ ID NO: 13, and/or wherein the mutation is at least a mutation of three point mutations of three consecutive positions within positions 6 to 13 with reference to SEQ ID NO: 13,
wherein the engineered Cas12a enzyme or the catalytically active fragment thereof has target strand (TS) nickase activity or non-target strand (NTS) nickase activity, preferably, wherein the engineered Cas12a enzyme or the catalytically active fragment thereof has non-target strand (NTS) nickase activity,
wherein the engineered Cas12a enzyme comprises or has an amino acid molecule according to SEQ ID NOs: 14 to 21 or 56, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the corresponding reference sequence, or wherein the engineered Cas12a enzyme at least comprises the core lid domain of any one of SEQ ID NOs: 14 to 21 or 56 starting at position 927, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the corresponding core lid domain, or
wherein the Cas12a enzyme having nickase activity comprises at least one further mutation, wherein the at least one further modification modifies the PAM-specificity and/or the thermotolerance of the engineered Cas12a enzyme.

3. (canceled)

4. (canceled)

5. (canceled)

6. (canceled)

7. (canceled)

8. A complex, or at least one nucleic acid molecule encoding the components of the complex, the complex comprising at least one engineered Cas12a enzyme having nickase activity or a catalytically active fragment of claim 1, and at least one compatible guide RNA, optionally comprising at least one further polypeptide, covalently and/or non-covalently attached to the at least one engineered Cas12a enzyme having nickase activity or the catalytically active fragment thereof within the complex, wherein the at least one further polypeptide is selected from an organellar localization sequence, including a nuclear localization signal (NLS), a mitochondrion localization signal, or a chloroplast localization signal, and/or wherein the at least one further polypeptide is a cell-penetrating polypeptide, preferably, in case the at least one further polypeptide is covalently attached to the at least one engineered Cas12a enzyme having nickase activity or the catalytically active fragment thereof, wherein the at least one further polypeptide is covalently attached to the N-terminus and/or the C-terminus of the at least one engineered Cas12a enzyme having nickase activity.

9. A fusion protein or at least one nucleic acid molecule encoding the same, comprising at least one engineered Cas12a enzyme having nickase activity or the catalytically active fragment thereof of claim 1, covalently and/or non-covalently attached to at least one further polypeptide domain, the at least one further polypeptide domain having an activity selected from an enzymatic activity, binding activity or targeting activity, and optionally comprising at least one guide RNA compatible with the engineered Cas12a enzyme having nickase activity, wherein the at least one compatible guide RNA covalently and/or non-covalently interacts with the at least one engineered Cas12a enzyme having nickase activity or the catalytically active fragment thereof.

10. An adenine or a cytidine base editor, or a base editor complex, or at least one nucleic acid molecule encoding the same, the base editor or base editor complex comprising at least one catalytically active portion of at least one engineered Cas12a enzyme having nickase activity of claim 1.

11. A prime editor or a prime editor complex, or at least one nucleic acid molecule encoding the same, the prime editor or prime editor complex comprising at least one catalytically active portion of at least one engineered Cas12a enzyme having nickase activity of claim 1.

12. A kit comprising
(i) an engineered Cas12a enzyme having nickase activity (nCas12a), or a catalytically active fragment thereof as defined in claim 1 or 2, or a complex as defined in claim 8, or at least one sequence encoding the same, or a of fusion protein as defined in claim 9, or at least one sequence encoding the same, or an adenine or a cytidine base editor, or a base editor complex, or at least one nucleic acid molecule encoding the same as defined in claim 10, or prime editor or a prime editor complex, or at least one nucleic acid molecule encoding the same as defined in claim 11;
(ii) at least one compatible guide RNA, or a set of compatible guide RNAs, each guide RNA being complementary to target sequences of interest; and
(iii) a set of reagents;
(iv) optionally comprising particles, vesicles, or at least one vector, including viral vectors, for assisting delivery, wherein said particles comprise a lipid, including lipid nanoparticles, a sugar, a metal or a polypeptide, or a combination thereof, or wherein said vesicles comprise exosomes or liposomes.

13. A nucleic acid molecule encoding a Cas12a enzyme having nickase activity, or a catalytically active fragment thereof,
wherein the nucleic acid molecule is codon-optimized for a fungal cell, including a yeast cell, a prokaryotic cell, including a Gram-positive, Gram-negative or Gram-variable bacterial cell, or an archaeal cell, and, optionally, comprises a nucleic acid molecule encoding at least one guide RNA,
wherein the Cas12a enzyme comprises at least one mutation in its core lid domain, and the mutation in the core lid domain is selected from:
(i) at least three point mutations of three consecutive positions within the core lid domain; or
(ii) a deletion of at least two consecutive positions within the core lid domain; or
(iii) a combination of at least one first point mutation at at least one position within the core lid domain and
(iiia) at least one deletion of at least one position within the core lid domain, and/or
(iiib) at least one, preferably at least two, at least three, or at least four further point mutation(s) at a different position in comparison to the first point mutation within the core lid domain, wherein the position(s) of the further point mutation(s) is/are not in consecutive order with the position(s) of the at least one first point mutation;
(iv) one point mutation at a position within the core lid domain;
wherein the at least one mutation in the core lid domain confers broad spectrum nickase activity, wherein the core lid domain reference sequence comprises a sequence as defined in SEQ ID NO: 13, optionally a complex additionally comprising at least one compatible guide RNA, or a sequence encoding the same, forming a complex with the cognate engineered Cas12a enzyme having nickase activity, or the catalytically active fragment thereof.

14. The nucleic acid molecule of claim 13, wherein the nucleic acid molecule is codon-optimized for a fungal cell, including a yeast cell,
wherein the nucleic acid molecule is codon-optimized for a prokaryotic cell, including a Gram-positive, Gram-negative or Gram-variable bacterial cell, or an archaeal cell,
wherein the nucleic acid molecule is codon-optimized for Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corvnebacterium acetophilum, Corvnebacterium glutamicum, Corvnebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, including Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp,
wherein the nucleic acid molecule is codon-optimized for Saccharomyces spec, including Saccharomyces cerevisiae, Hansenula spec, including Hansenula polymorpha, Schizosaccharomyces spec, including Schizosaccharomyces pombe, Kluyveromyces spec, including Kluyveromyces lactis and Kluvveromyces marxianus, Yarrowia spec, including Yarrowia lipolytica, Pichia spec, including Pichia methanolica, Pichia stipites and Pichia pastoris, Zygosaccharomyces spec, including Zygosaccharomyces rouxii and Zygosaccharomyces bailii, Candida spec, including Candida boidinii, Candida utilis, Candida freyschussii, Candida glabrata and Candida sonorensis, Schwanniomyces spec, including Schwanniomyces occidentalis, Arxula spec, including Arxula adeninivorans, Ogataea spec including Ogataea minuta, Aspergillus spec, including Aspergillus niger or Myceliophthora thermophila, or
wherein the nucleic acid molecule comprises or consists of a sequence according to SEQ ID NOs: 80 to 87, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99%.

15. (canceled)

16. (canceled)

17. (canceled)

18. (canceled)

19. An expression construct or vector comprising at least one nucleic acid molecule of claim 13.

20. A kit comprising
(i) an expression construct or vector as defined in claim 19;
(ii) at least one compatible guide RNA, or a set of compatible guide RNAs, each guide RNA being complementary to target sequences of interest; and
(iii) a set of reagents;
(iv) optionally comprising particles, vesicles, or at least one vector, including viral vectors, for assisting delivery, wherein said particles comprise a lipid, including lipid nanoparticles, a sugar, a metal or a polypeptide, or a combination thereof, or wherein said vesicles comprise exosomes or liposomes.

21. A fungal cell, including a yeast cell, or a prokaryotic cell, including a Gram-positive, Gram-negative or Gram-variable bacterial cell, preferably a Gram-negative bacterial cell, or an archaeal cell comprising at least one nucleic acid molecule of claim 13; and/or at least one expression construct or vector of claim 19; and/or an engineered Cas12a enzyme having nickase activity, or a catalytically active fragment thereof, the engineered Cas12a enzyme comprising at least one mutation in its core lid domain, wherein the mutation in the core lid domain is selected from:
(i) at least three point mutations of three consecutive positions within the core lid domain; or
(ii) a deletion of at least two consecutive positions within the core lid domain; or
(iii) a combination of at least one first point mutation at at least one position within the core lid domain and
(iiia) at least one deletion of at least one position within the core lid domain, and/or
(iiib) at least one, preferably at least two, at least three, or at least four further point mutation(s) at a different position in comparison to the first point mutation within the core lid domain, wherein the position(s) of the further point mutation(s) is/are not in consecutive order with the position(s) of the at least one first point mutation;
(iv) one point mutation at a position within the core lid domain;
wherein the at least one mutation in the core lid domain confers broad spectrum nickase activity, wherein the core lid domain reference sequence comprises a sequence as defined in SEQ ID NO: 13, optionally a complex additionally comprising at least one compatible guide RNA, or a sequence encoding the same, forming a complex with the cognate engineered Cas12a enzyme having nickase activity, or the catalytically active fragment thereof.

22. The cell of claim 21, wherein the cell is a fungal cell, including a yeast cell, preferably wherein the fungal cell, including the yeast cell,
wherein the cell is selected from a cell originating from to Saccharomyces spec, including Saccharomyces cerevisiae, Hansenula spec, including Hansenula polymorpha, Schizosaccharomyces spec, including Schizosaccharomyces pombe, Kluyveromyces spec, including Kluyveromyces lactis and Kluvveromyces marxianus, Yarrowia spec, including Yarrowia lipolytica, Pichia spec, including Pichia methanolica, Pichia stipites and Pichia pastoris, Zygosaccharomyces spec, including Zygosaccharomyces rouxii and Zygosaccharomyces bailii, Candida spec, including Candida boidinii, Candida utilis, Candida freyschussii, Candida glabrata and Candida sonorensis, Schwanniomyces spec, including Schwanniomyces occidentalis, Arxula spec, including Arxula adeninivorans, Ogataea spec including Ogataea minuta, Aspergillus spec. including Aspergillus niger or Myceliophthora thermophila,
wherein the cell is a prokaryotic cell, including a Gram-positive, Gram-negative or Gram-variable bacterial cell, or
wherein the cell is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, including Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp.

23. (canceled)

24. (canceled)

25. (canceled)

26. A method for modifying the genomic locus of interest of at least one fungal cell, including a yeast cell, or a prokaryotic cell, including a Gram-positive, Gram negative or Gram-variable bacterial cell, preferably a Gram-negative bacterial cell, or archaeal cell, or at least one construct at or near at least one target site, the method comprising:
(a) providing at least one cell or construct comprising the genomic locus to be modified;
(b) providing and/or introducing
(i) at least one engineered Cas12a enzyme having nickase activity (nCas12a), or a catalytically active fragment thereof, or at least one nucleic acid molecule encoding the same, as defined in claim 1 or 2; or
(ii) at least one complex or at least one nucleic acid molecule encoding the same as defined in claim 8; or at least one fusion protein or at least one nucleic acid molecule encoding the same as defined in claim 9; or
(iii) at least one adenine or a cytidine base editor, or at least one base editor complex, or at least one nucleic acid molecule encoding the same as defined in claim 10; or
(iv) at least one prime editor or at least one prime editor complex, or at least one nucleic acid molecule encoding the same as defined in claim 11; or
(v) at least one expression construct or vector as defined in claim 19;
to/into the at least one cell or construct;
(c) providing and/or introducing at least one compatible guide RNA or a sequence encoding the same, as defined in claim 1;
(d) allowing complex formation of the at least one engineered Cas12a enzyme having nickase activity, or the catalytically active fragment thereof of (a) and the at least compatible guide RNA as defined in claim (b) and thus allowing the insertion of at least one nick at the genomic locus of interest of the at least one cell or construct at or near at least one target site;
(e) optionally: providing at least one donor repair template, or at least one the nucleic acid molecule encoding the same; and
(f) obtaining at least one edited cell or construct comprising a modification of a genomic locus of interest at or near a target site;
optionally, where the method comprises the following step:
(g) regenerating at least one population of edited cells, tissues, organs, materials or whole organisms from the at least one edited cell or construct.

27. The method of claim 26, wherein the method is performed in vitro or in vivo,
wherein the cell or construct originates from a fungal cell, including a yeast cell,
wherein the fungal cell, including the yeast cell, is selected from a cell originating from Saccharomyces spec, including Saccharomyces cerevisiae, Hansenula spec, including Hansenula polymorpha, Schizosaccharomyces spec, including Schizosaccharomyces pombe, Kluyveromyces spec, including Kluyveromyces lactis and Kluyveromyces marxianus, Yarrowia spec, including Yarrowia lipolytica, Pichia spec, including Pichia methanolica, Pichia stipites and Pichia pastoris, Zygosaccharomyces spec, including Zygosaccharomyces rouxii and Zygosaccharomyces bailii, Candida spec, including Candida boidinii, Candida utilis, Candida freyschussii, Candida glabrata and Candida sonorensis, Schwanniomyces spec, including Schwanniomyces occidentalis, Arxula spec, including Arxula adeninivorans, Ogataea spec including Ogataea minuta, Aspergillus spec. including Aspergillus niger or Myceliophthora thermophile,
wherein the cell or construct originates from a prokaryotic cell, including a Gram-positive, Gram-negative or Gram-variable bacterial cell, preferably a Gram-negative bacterial cells, or an archaeal cell,
wherein the wherein the prokaryotic cell is selected from a cell originating from Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, Brevibacterium divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Klebsiella spec, including Klebsiella pneumonia, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas jluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces avermitilis, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Escherichia freundii, Microbacterium ammoniaphilum, Serratia marcescens, Salmonella typhimurium, Salmonella schottmulleri, Xanthomonas citri, Synechocystis sp., Synechococcus elongatus, Thermosynechococcus elongatus, Microcystis aeruginosa, Nostoc sp., N. commune, N. sphaericum, Nostoc punctiforme, Spirulina platensis, Lyngbya majuscula, L. lagerheimii, Phormidium tenue, Anabaena sp., or Leptolyngbya sp,
wherein the modification is at least one insertion, at least one deletion, or at least one point mutation,
wherein, during step (a) to (c), at least one additional effector, or a nucleic acid molecule encoding the same, is provided, the additional effector promoting DNA repair and cell regeneration before, during or upon insertion of at least one nick at the genomic locus of interest at or near at least one target site,
wherein the method is a concerted double-nicking method, wherein at least two Cas enzymes having nickase activity (nCas), or catalytically active fragments thereof, or at least one nucleic acid molecule encoding the same, are provided in step (a); and wherein in step (c) at least two compatible guide RNAs are provided, wherein the at least two compatible guide RNAs are designed to allow a concerted action of the at least two Cas enzymes having nickase activity so that the at least two Cas enzymes having nickase activity introduce two individual nicks at the at least one target site,
wherein the two Cas enzymes having nickase activity, or the catalytically active fragments thereof, can be the same or different, wherein at least one of the at least two Cas enzymes having nickase activity, or the catalytically active fragment thereof, is an engineered Cas12a enzyme having nickase activity (nCas12a), or a catalytically active fragment thereof, or the sequence encoding the same, as defined in claims 1 to 2, wherein the nCas12a can be the same nCas12a, or a different nCas12a,
wherein two individual nicks are introduced into opposite strands within the genomic locus of interest of the at least one cell or construct at or near the at least one target site, wherein the offset is positive, negative, or zero, preferably wherein the offset is between around −100 bp and +100 bp, or
wherein the two Cas enzymes having nickase activity and/or the at least two compatible guide RNAs are individually provided in the form of at least one expression construct or vector, or in the form of at least one complex, or in the form of at least one nucleic acid molecule encoding the same, or in the form of at least one of fusion proteins or at least one nucleic acid molecule encoding the same.

28. (canceled)

29. (canceled)

30. (canceled)

31. (canceled)

32. (canceled)

33. (canceled)

34. (canceled)

35. (canceled)

36. (canceled)

37. (canceled)

38. An edited cell, tissue, organ, material or whole organism obtained by or obtainable by a method according to claim 26 to 27.

39. A plant cell comprising an engineered Cas12a enzyme having nickase activity (nCas12a), or a catalytically active fragment thereof, the engineered Cas12a enzyme comprising at least one mutation in its core lid domain, wherein the mutation in the core lid domain is selected from:
(i) at least three point mutations of three consecutive positions within the core lid domain; or
(ii) a deletion of at least two consecutive positions within the core lid domain; or
(iii) a combination of at least one first point mutation at at least one position within the core lid domain and
(iiia) at least one deletion of at least one position within the core lid domain, and/or
(iiib) at least one, preferably at least two, at least three, or at least four further point mutation(s) at a different position in comparison to the first point mutation within the core lid domain, wherein the position(s) of the further point mutation(s) is/are not in consecutive order with the position(s) of the at least one first point mutation;
(iv) one point mutation at a position within the core lid domain;
wherein the at least one mutation in the core lid domain confers broad spectrum nickase activity, wherein the core lid domain reference sequence comprises a sequence as defined in SEQ ID NO: 13, optionally a complex additionally comprising at least one compatible guide RNA, or a sequence encoding the same, forming a complex with the cognate engineered Cas12a enzyme having nickase activity, or the catalytically active fragment thereof.

40. The plant cell of claim 39, wherein the engineered Cas12a enzyme is based on a wild-type Cas12a sequence according to any one of SEQ ID NOs: 1 to 12, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the corresponding wild-type sequence as reference sequence, or an ortholog or homolog of a sequence according to any one of SEQ ID NOs: 1 to 12 having at least 95%, 96%, 97%, 98% or at least 99% sequence identity to the corresponding ortholog or homolog sequence as reference sequence,
wherein the at least three point mutations in three consecutive amino acids are positioned within positions 2 to 16 with reference to SEQ ID NO: 13, and/or wherein the deletion is a deletion of at least two, at least three, at least four, at least five, at least six at least seven, at least eight, at least nine, at least ten, at least eleven, at least twelve, at least thirteen, at least fourteen, at least fifteen, at least sixteen, or at least seventeen consecutive positions within the core lid domain,
wherein the mutation is a deletion of at least four, at least five, at least six at least seven, or at least all eight positions 6 to 13 with reference to SEQ ID NO: 13, and/or wherein the mutation is at least a mutation of three point mutations of three consecutive positions within positions 6 to 13 with reference to SEQ ID NO: 13,
wherein the engineered Cas12a enzyme or the catalytically active fragment thereof has target strand (TS) nickase activity or non-target strand (NTS) nickase activity, preferably, wherein the engineered Cas12a enzyme or the catalytically active fragment thereof has non-target strand (NTS) nickase activity,
wherein the engineered Cas12a enzyme comprises or has an amino acid sequence according to SEQ ID NOs: 14 to 21 or 56 or 100 to 106, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the corresponding reference sequence, or wherein the engineered Cas12a enzyme at least comprises the core lid domain of any one of SEO ID NOs: 14 to 21 or 56 or 100 to 106 starting at position 927, or a sequence having at least 75%, 76%, 77%, 78%, 79%, 80%, 81%, 82%, 83%, 84%, 85%, 86%, 87%, 88%, 89%, 900, 910, 92%, 93%, 94%, 95%, 96%, 97%, 98%, or at least 99% sequence identity to the corresponding core lid domain, or
wherein the Cas12a enzyme having nickase activity comprises at least one further mutation, wherein the at least one further modification modifies the PAM-specificity and/or the thermotolerance of the engineered Cas12a enzyme.

41. (canceled)

42. (canceled)

43. (canceled)

44. (canceled)

45. (canceled)

46. A nucleic acid molecule encoding the Cas12a enzyme or the catalytically active fragment thereof of claim 39, wherein the nucleic acid molecule is codon-optimized for a plant cell and, optionally, comprises a nucleic acid molecule encoding at least one guide RNA,
wherein the nucleic acid molecule is codon-optimized for a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp, or
wherein the nucleic acid molecule comprises or consists of a sequence according to SEQ ID NOs: 88 to 93, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99%.

47. (canceled)

48. (canceled)

49. An expression construct or vector comprising at least one nucleic acid molecule of claim 46.

50. A plant cell comprising at least one nucleic acid molecule of claim 46; and/or at least one expression construct or vector of claim 49.

51. The plant cell of claim 39 or claim 50, wherein the cell is selected from a cell originating from a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp.

52. A complex, or at least one nucleic acid molecule encoding the components of the complex, the complex comprising at least one engineered Cas12a enzyme having nickase activity or a catalytically active fragment of claim 39, and at least one compatible guide RNA, optionally comprising at least one further polypeptide, covalently and/or non-covalently attached to the at least one engineered Cas12a enzyme having nickase activity or the catalytically active fragment thereof within the complex, wherein the at least one further polypeptide is selected from an organellar localization sequence, including a nuclear localization signal (NLS), a mitochondrion localization signal, or a chloroplast localization signal, and/or wherein the at least one further polypeptide is a cell-penetrating polypeptide, preferably, in case the at least one further polypeptide is covalently attached to the at least one engineered Cas12a enzyme having nickase activity or the catalytically active fragment thereof, wherein the at least one further polypeptide is covalently attached to the N-terminus and/or the C-terminus of the at least one engineered Cas12a enzyme having nickase activity.

53. A fusion protein or at least one nucleic acid molecule encoding the same, comprising at least one engineered Cas12a enzyme having nickase activity or the catalytically active fragment thereof of claim 39, covalently and/or non-covalently attached to at least one further polypeptide domain, the at least one further polypeptide domain having an activity selected from an enzymatic activity, binding activity or targeting activity, and optionally comprising at least one guide RNA compatible with the engineered Cas12a enzyme having nickase activity, wherein the at least one compatible guide RNA covalently and/or non-covalently interacts with the at least one engineered Cas12a enzyme having nickase activity or the catalytically active fragment thereof.

54. An adenine or a cytidine base editor, or a base editor complex, or at least one nucleic acid molecule encoding the same, the base editor or base editor complex comprising at least one catalytically active portion of at least one engineered Cas12a enzyme having nickase activity of claim 39.

55. A prime editor or a prime editor complex, or at least one nucleic acid molecule encoding the same, the prime editor or prime editor complex comprising at least one catalytically active portion of at least one engineered Cas12a enzyme having nickase activity of claim 39.

56. A kit comprising
(i) an engineered Cas12a enzyme having nickase activity (nCas12a), or a catalytically active fragment thereof as defined in claim 39 or 40, or an expression construct or vector as defined in claim 49, or a complex as defined in claim 52, or at least one sequence encoding the same, or a of fusion protein as defined in claim 53, or at least one sequence encoding the same, or an adenine or a cytidine base editor, or a base editor complex, or at least one nucleic acid molecule encoding the same as defined in claim 54, or prime editor or a prime editor complex, or at least one nucleic acid molecule encoding the same as defined in claim 55;
(ii) at least one compatible guide RNA, or a set of compatible guide RNAs, each guide RNA being complementary to target sequences of interest; and
(iii) a set of reagents;
(iv) optionally comprising particles, vesicles, or at least one vector, including viral and/or Agrobacterium vector, for assisting delivery, wherein said particles comprise a lipid, including lipid nanoparticles, a sugar, a metal or a polypeptide, or a combination thereof, or wherein said vesicles comprise exosomes or liposomes.

57. A method for modifying the genomic locus of interest of at least one plant cell at or near at least one target site, the method comprising:
(a) providing at least one plant cell comprising the genomic locus to be modified;
(b) introducing
(i) at least one engineered Cas12a enzyme having nickase activity (nCas12a), or a catalytically active fragment thereof, or at least one nucleic acid molecule encoding the same, as defined in claim 39 or 40; or
(ii) at least one expression construct or vector as defined in claim 49; or
(iii) at least one complex or at least one nucleic acid molecule encoding the same as defined in claim 52; or
at least one fusion protein or at least one nucleic acid molecule encoding the same as defined in claim 53; or
(iv) at least one adenine or a cytidine base editor, or at least one base editor complex, or at least one nucleic acid molecule encoding the same as defined in claim 54; or
(v) at least one prime editor or at least one prime editor complex, or at least one nucleic acid molecule encoding the same as defined in claim 55;
into the at least one plant cell;
(c) introducing at least one compatible guide RNA or a sequence encoding the same, as defined in claim 39;
(d) allowing complex formation of the at least one engineered Cas12a enzyme having nickase activity, or the catalytically active fragment thereof of (a) and the at least compatible guide RNA as defined in claim (b) and thus allowing the insertion of at least one nick at the genomic locus of interest of the at least one cell or construct at or near at least one target site;
(e) optionally: providing at least one donor repair template, or at least one the nucleic acid molecule encoding the same; and
(f) obtaining at least one edited plant cell comprising a modification of a genomic locus of interest at or near a target site;
optionally, where the method comprises the following step:
(g) regenerating at least one population of edited plant cells, tissues, organs, materials or whole organisms from the at least one edited cell or construct.

58. The method of claim 57, wherein the plant cell is selected from a cell originating from a plant which belongs to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, or Ziziphus spp, preferably wherein the plant cell is selected from a cell originating from Glycine max, a Zea mays, a Brassica napus, a Gossypium spp. an Oryza sativa or Triticum aestivum.
wherein the modification is at least one insertion, at least one deletion, or at least one point mutation,
wherein, during step (a) to (c), at least one additional effector, or a nucleic acid molecule encoding the same, is provided, the additional effector promoting DNA repair and cell regeneration before, during or upon insertion of at least one nick at the genomic locus of interest at or near at least one target site,
wherein the method is a concerted double-nicking method, wherein at least two Cas enzymes having nickase activity (nCas), or catalytically active fragments thereof, or at least one nucleic acid molecule encoding the same, are provided in step (a); and wherein in step (c) at least two compatible guide RNAs are provided, wherein the at least two compatible guide RNAs are designed to allow a concerted action of the at least two Cas enzymes having nickase activity so that the at least two Cas enzymes having nickase activity introduce two individual nicks at the at least one target site,
wherein the two Cas enzymes having nickase activity, or the catalytically active fragments thereof, can be the same or different, wherein at least one of the at least two Cas enzymes having nickase activity, or the catalytically active fragment thereof, is an engineered Cas12a enzyme having nickase activity (nCas12a), or a catalytically active fragment thereof, or the sequence encoding the same, as defined in claim 39 or 40, wherein the nCas12a can be the same nCas12a, or a different nCas12a,
wherein two individual nicks are introduced into opposite strands within the genomic locus of interest of the at least one cell or construct at or near the at least one target site, wherein the offset is positive, negative, or zero, preferably wherein the offset is between around −100 bp and +100 bp, or
wherein the two Cas enzymes having nickase activity and/or the at least two compatible guide RNAs are individually provided in the form of at least one expression construct or vector, or in the form of at least one complex, or in the form of at least one nucleic acid molecule encoding the same, or in the form of at least one of fusion proteins or at least one nucleic acid molecule encoding the same.

59. (canceled)

60. (canceled)

61. (canceled)

62. (canceled)

63. (canceled)

64. (canceled)

65. An edited plant cell, tissue, organ, material or whole organism obtained by or obtainable by a method according to claim 57 or 58.

66. An animal cell, including a human cell, comprising an engineered Cas12a enzyme having nickase activity (nCas12a), or a catalytically active fragment thereof, the engineered Cas12a enzyme comprising at least one mutation in its core lid domain, wherein the mutation in the core lid domain is selected from:
(i) at least three point mutations of three consecutive positions within the core lid domain; or
(ii) a deletion of at least two consecutive positions within the core lid domain; or
(iii) a combination of at least one first point mutation at at least one position within the core lid domain and
(iiia) at least one deletion of at least one position within the core lid domain, and/or
(iiib) at least one, preferably at least two, at least three, or at least four further point mutation(s) at a different position in comparison to the first point mutation within the core lid domain, wherein the position(s) of the further point mutation(s) is/are not in consecutive order with the position(s) of the at least one first point mutation;
(iv) one point mutation at a position within the core lid domain;
wherein the at least one mutation in the core lid domain confers broad spectrum nickase activity, wherein the core lid domain reference sequence comprises a sequence as defined in SEQ ID NO: 13, optionally a complex additionally comprising at least one compatible guide RNA, or a sequence encoding the same, forming a complex with the cognate engineered Cas12a enzyme having nickase activity, or the catalytically active fragment thereof.

67. The animal cell of claim 66, wherein the engineered Cas12a enzyme is based on a wild-type Cas12a sequence according to any one of SEQ ID NOs: 1 to 12, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the corresponding wild-type sequence as reference sequence, or an ortholog or homolog of a sequence according to any one of SEQ ID NOs: 1 to 12 having at least 95%, 96%, 97%, 98% or at least 99% sequence identity to the corresponding ortholog or homolog sequence as reference sequence,
wherein the at least three point mutations in three consecutive amino acids are positioned within positions 2 to 16 with reference to SEQ ID NO: 13, and/or wherein the deletion is a deletion of at least two, at least three, at least four, at least five, at least six at least seven, at least eight, at least nine, at least ten, at least eleven, at least twelve, at least thirteen, at least fourteen, at least fifteen, at least sixteen, or at least seventeen consecutive positions within the core lid domain,
wherein the mutation is a deletion of at least four, at least five, at least six at least seven, or at least all eight positions 6 to 13 with reference to SEQ ID NO: 13, and/or wherein the mutation is at least a mutation of three point mutations of three consecutive positions within positions 6 to 13 with reference to SEQ ID NO: 13,
wherein the engineered Cas12a enzyme or the catalytically active fragment thereof has target strand (TS) nickase activity or non-target strand (NTS) nickase activity, preferably, wherein the engineered Cas12a enzyme or the catalytically active fragment thereof has non-target strand (NTS) nickase activity,
wherein the engineered Cas12a enzyme comprises or has an amino acid molecule according to SEQ ID NOs: 14 to 21 or 56 or 100 to 106, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99% sequence identity to the corresponding reference sequence, or wherein the engineered Cas12a enzyme at least comprises the core lid domain of any one of SEQ ID NOs: 14 to 21 or 56 or 100 to 106 starting at position 927, or a sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to the corresponding core lid domain, or
wherein the Cas12a enzyme having nickase activity comprises at least one further mutation, wherein the at least one further modification modifies the PAM-specificity and/or the thermotolerance of the engineered Cas12a enzyme.

68. (canceled)

69. (canceled)

70. (canceled)

71. (canceled)

72. (canceled)

73. A nucleic acid molecule encoding the Cas12a enzyme or the catalytically active fragment thereof of claim 66, wherein the nucleic acid molecule is codon-optimized for an animal cell, including a human cell, and, optionally, comprises a nucleic acid molecule encoding at least one guide RNA,
wherein the nucleic acid molecule is codon optimized for an insect, poultry, fish, crustacea, or mammalian cell, preferably for a mammalian cell: optionally being selected from a cell originating from a non-human primate, bovine, porcine, rodent, including mouse or rat, or human cell, or
wherein the nucleic acid molecule comprises or consists of a sequence according to SEQ ID NOs: 94 to 99, or a sequence having at least 75%, 76%, 77%, 78%, 79%, 80%, 81%, 82%, 83%, 840%, 850%, 860%, 870%, 880%, 890%, 900%, 910%, 920%, 93%, 94%, 95%, 960%, 97%, 98% or at least 99%.

74. (canceled)

75. (canceled)

76. An expression construct or vector comprising at least one nucleic acid molecule of claim 73.

77. An animal cell, including a human cell, comprising at least one nucleic acid molecule of claim 73; and/or at least one expression construct or vector of claim 76.

78. The animal cell of claim 66 or 77, wherein the cell is an insect, poultry, fish, crustacea, or a mammalian cell, preferably wherein the cell is a mammalian cell; optionally being selected from a cell originating from a non-human primate, bovine, porcine, rodent, including mouse or rat, or human cell.

79. A complex, or at least one nucleic acid molecule encoding the components of the complex, the complex comprising at least one engineered Cas12a enzyme having nickase activity or a catalytically active fragment of claim 66, and at least one compatible guide RNA, optionally comprising at least one further polypeptide, covalently and/or non-covalently attached to the at least one engineered Cas12a enzyme having nickase activity or the catalytically active fragment thereof within the complex, wherein the at least one further polypeptide is selected from an organellar localization sequence, including a nuclear localization signal (NLS), a mitochondrion localization signal, or a chloroplast localization signal, and/or wherein the at least one further polypeptide is a cell-penetrating polypeptide, preferably, in case the at least one further polypeptide is covalently attached to the at least one engineered Cas12a enzyme having nickase activity or the catalytically active fragment thereof, wherein the at least one further polypeptide is covalently attached to the N-terminus and/or the C-terminus of the at least one engineered Cas12a enzyme having nickase activity.

80. A fusion protein or at least one nucleic acid molecule encoding the same, comprising at least one engineered Cas12a enzyme having nickase activity or the catalytically active fragment thereof of claim 66, covalently and/or non-covalently attached to at least one further polypeptide domain, the at least one further polypeptide domain having an activity selected from an enzymatic activity, binding activity or targeting activity, and optionally comprising at least one guide RNA compatible with the engineered Cas12a enzyme having nickase activity, wherein the at least one compatible guide RNA covalently and/or non-covalently interacts with the at least one engineered Cas12a enzyme having nickase activity or the catalytically active fragment thereof.

81. An adenine or a cytidine base editor, or a base editor complex, or at least one nucleic acid molecule encoding the same, the base editor or base editor complex comprising at least one catalytically active portion of at least one engineered Cas12a enzyme having nickase activity of claim 66.

82. A prime editor or a prime editor complex, or at least one nucleic acid molecule encoding the same, the prime editor or prime editor complex comprising at least one catalytically active portion of at least one engineered Cas12a enzyme having nickase activity of claim 66.

83. A kit comprising
(i) an engineered Cas12a enzyme having nickase activity (nCas12a), or a catalytically active fragment thereof as defined in claim 66 or 67, or an expression construct or vector as defined in claim 76, or a complex as defined in claim 79, or at least one sequence encoding the same, or a of fusion protein as defined in claim 80, or at least one sequence encoding the same, or an adenine or a cytidine base editor, or a base editor complex, or at least one nucleic acid molecule encoding the same as defined in claim 81, or prime editor or a prime editor complex, or at least one nucleic acid molecule encoding the same as defined in claim 82;
(ii) at least one compatible guide RNA, or a set of compatible guide RNAs, each guide RNA being complementary to target sequences of interest; and
(iii) a set of reagents;
(iv) optionally comprising particles, vesicles, or at least one viral vector for assisting delivery, wherein said particles comprise a lipid, including lipid nanoparticles, a sugar, a metal or a polypeptide, or a combination thereof, or wherein said vesicles comprise exosomes or liposomes.

84. A method for modifying the genomic locus of interest of at least one animal cell, including human cell, at or near at least one target site, the method comprising:
(a) providing at least one animal cell comprising the genomic locus to be modified;
(b) introducing
(i) at least one engineered Cas12a enzyme having nickase activity (nCas12a), or a catalytically active fragment thereof, or at least one nucleic acid molecule encoding the same, as defined in claim 66 or 67; or
(ii) at least one expression construct or vector as defined in claim 76; or
(iii) at least one complex or at least one nucleic acid molecule encoding the same as defined in claim 79; or
at least one fusion protein or at least one nucleic acid molecule encoding the same as defined in claim 80; or
(iv) at least one adenine or a cytidine base editor, or at least one base editor complex, or at least one nucleic acid molecule encoding the same as defined in claim 81; or
(v) at least one prime editor or at least one prime editor complex, or at least one nucleic acid molecule encoding the same as defined in claim 82;
into the at least one animal cell;
(c) introducing at least one compatible guide RNA or a sequence encoding the same, as defined in claim 66;
(d) allowing complex formation of the at least one engineered Cas12a enzyme having nickase activity, or the catalytically active fragment thereof of (a) and the at least compatible guide RNA as defined in claim (b) and thus allowing the insertion of at least one nick at the genomic locus of interest of the at least one cell or construct at or near at least one target site;
(e) optionally: providing at least one donor repair template, or at least one the nucleic acid molecule encoding the same; and
(f) obtaining at least one edited animal cell, including human cell, comprising a modification of a genomic locus of interest at or near a target site;
wherein the method excludes processes for modifying the germ line genetic identity of human beings, uses of human embryos for industrial or commercial purposes and processes for modifying the genetic identity of animals which are likely to cause them suffering without any substantial medical benefit to man or animal, and also animals resulting from such processes,
optionally, where the method comprises the following step:
(g) regenerating at least one population of edited cells, tissues, organs, materials or whole organisms from the at least one edited animal cell, including human cell.

85. The method of claim 84, wherein the animal cell is an insect, poultry, fish, crustacea, or a mammalian cell, preferably wherein the cell originates a mammalian cell, optionally being selected from a cell originating from a non-human primate, bovine, porcine, rodent, including mouse or rat, or human cell,
wherein the modification is at least one insertion, at least one deletion, or at least one point mutation, or
wherein, during step (a) to (c), at least one additional effector, or a nucleic acid molecule encoding the same, is provided, the additional effector promoting DNA repair and cell regeneration before, during or upon insertion of at least one nick at the genomic locus of interest at or near at least one target site.

86. (canceled)

87. (canceled)

88. The method of claim 84, wherein the method is a concerted double-nicking method, wherein at least two Cas enzymes having nickase activity (nCas), or catalytically active fragments thereof, or at least one nucleic acid molecule encoding the same, are provided in step (a); and wherein in step (c) at least two compatible guide RNAs are provided, wherein the at least two compatible guide RNAs are designed to allow a concerted action of the at least two Cas enzymes having nickase activity so that the at least two Cas enzymes having nickase activity introduce two individual nicks at the at least one target site,
wherein the two Cas enzymes having nickase activity, or the catalytically active fragments thereof, can be the same or different, wherein at least one of the at least two Cas enzymes having nickase activity, or the catalytically active fragment thereof, is an engineered Cas12a enzyme having nickase activity (nCas12a), or a catalytically active fragment thereof, or the sequence encoding the same, as defined in claim 66 or 67, wherein the nCas12a can be the same nCas12a, or a different nCas12a,
wherein two individual nicks are introduced into opposite strands within the genomic locus of interest of the at least one cell or construct at or near the at least one target site, wherein the offset is positive, negative, or zero, preferably wherein the offset is between around −100 bp and +100 bp, or
wherein the two Cas enzymes having nickase activity and/or the at least two compatible guide RNAs are individually provided in the form of at least one expression construct or vector, or in the form of at least one complex, or in the form of at least one nucleic acid molecule encoding the same, or in the form of at least one of fusion proteins or at least one nucleic acid molecule encoding the same.

89. (canceled)

90. (canceled)

91. (canceled)

92. An edited animal cell, tissue, organ, material or whole organism obtained by or obtainable by a method according to any one of claims 84, 85, or 88.

93. A method of treating or preventing a disease, the method comprising using
(i) at least one engineered Cas12a enzyme having nickase activity (nCas12a), or a catalytically active fragment thereof, or at least one nucleic acid molecule encoding the same, as defined in claim 66 or 67,
(ii) at least one expression construct or vector as defined in claim 76; or
(iii) at least one complex or at least one nucleic acid molecule encoding the same as defined in claim 79, or a fusion protein or at least one nucleic acid molecule encoding the same as defined in claim 80; or
(iv) at least one adenine or a cytidine base editor, or at least one base editor complex, or at least one nucleic acid molecule encoding the same as defined in claim 81; or
(v) at least one prime editor or at least one prime editor complex, or at least one nucleic acid molecule encoding the same as defined in claim 82; or
(vi) a kit as defined in claim 83; or
(vii) a cell as defined in claim 77 or 78; or
(viii) an edited cell, tissue, organ, material or whole organism as defined in claim 92;
for introducing at least one modification in a genomic locus of interest of at least one cell of a subject in need thereof at or near at least one disease-state related target site.

94. The method of claim 93, wherein the method comprises an ex vivo modification of the genomic locus, wherein at least one cell of a subject is provided to perform an ex vivo modification of the genomic locus to obtain at least one edited cell.

95. A method for cell therapy, comprising administering to a patient in need thereof, said edited at least one cell of claim 92, wherein presence of said edited cell remedies a disease in said patient.


SEQUENCE LISTING (145 sequences)

SEQ ID NO: 1 - protein, 1228 aa, unidentified
  source 1..1228: note = Lachnospiracea bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
  source 1..1307: note = BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1300 aa, Francisella tularensis
  source 1..1300: note = subsp. novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVISTAVLEYITQQVAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 4 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTLEEKSIKETLSLLFDDLKAQKLDLSKIYFKNDKSLTDLSQQVFDDYSVIGTAVLEYITQQVAPKNLDNPSKKEQELIAKKTEKAKYLSLETIKLALEEFNKHRDIDKQCRFEEILTNFAAIPMIFDEIAQNKDNLAQISLKYQNQGKKDLLQASAEDDVKAIKDLLDQTNNLLHRLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGNPQKGYQKFEFNIEDCRKFIDFYKESISKHPEWKDFGFRFSDTQRYNSIDEFYREVENQGYKLTFENISQDYINSLVNEGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINSLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVIFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 5 - protein, 1262 aa, Porphyromonas crevioricanis
MPWIDLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKVLPEYKEGEPYFEKMDYKFLPDPNKMLPKVFLSKKGIEIYEPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHAQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 6 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQAEKRKAIYKKFADDDRFKNMFSAKLISDILPEFVIHNNNYSASEKEEKTQVIKLFSRFATSFKDYFKNRANCFSADDISSSSCHRIVNDNAEIFFSNALVYRRIVKNLSNDDINKISGDIKDSLKEMSLEEIYSYEKYGEFITQEGISFYNDICGKVNLFMNLYCQKNKENKNLYKLRKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCPDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPWVFLSSKKGMETYKPSAYILEGYKQNKHLKSSKDFDITFCHDLIDYFKNCIAIHPEWKNFGFEFSDTSTYEDISGFYREVELQGYKIDWTYISEKDIELLQEKGQLYLFQIYNKDFSKKSTGNDNLHTMYLKNLFSEENLKDIVLKLNGKAEIFFRKSSIKNPIIHKKGSILVNRTYEAEEKEQFGNIRIVRKNIPENIYQELYKYYNDKSDKELSDEAAKLKDIVGHHEATKDIVKDYRYTYDKYFLHMPITINFKANKTSFINDRILQYIATEKELHVIGIDRGERNLIYVSVIDTCGNIVEQKSFNIVNGYDYQIKLKQQEGARQTARKEWKEIGKIKEIKEGYLSLVIHEISKMVIKYNAIIAMEDLSYGFKKGRFKVERQVYQKFETMLINKLNYLVFKDISITENGGLLKGYQLTYIPEKLKNVGHQCGCIFYVPAAYTSKIDPTTGFVNIFKFKDLTVDAKREFIKKFDSIRYDSDKNLFCFTFDYNNFITQNTVMSKSNWSVYTYGVRIKRRFVNGRFSNESDTIDITKDMEKTLEMTDINWRDGHDLRQDIIDYEIVQHIFEIFKLTVQMRNSLSELEDRNYDRLISPVLNENNIFYDSAKAGDALPKDADANGAYCIALKGLYEIKQITENWKEDGKFSRDKLKISNKDWFDFIQNKRYL

SEQ ID NO: 7 - protein, 1227 aa, Methanomethylophilus alvus
  source 1..1227: note = Candidatus Methanomethylophilus alvus
MDAKEFTGQYPLSKTLRFELRPIGRTWDNLEASGYLAEDRHRAECYPRAKELLDDNHRAFLNRVLPQIDMDWHPIAEAFCKVHKNPGNKELAQDYNLQLSKRRKEISAYLQDADGYKGLFAKPALDEAMKIAKENGNESDIEVLEAFNGFSVYFTGYHESRENIYSDEDMVSVAYRITEDNFPRFVSNALIFDKLNESHPDIISEVSGNLGVDDIGKYFDVSNYNNFLSQAGIDDYNHIIGGHTTEDGLIQAFNVVLNLRHQKDPGFEKIQFKQLYKQILSVRTSKSYIPKQFDNSKEMVDCICDYVSKIEKSETVERALKLVRNISSFDLRGIFVNKKNLRILSNKLIGDWDAIETALMHSSSSENDKKSVYDSAEAFTLDDIFSSVKKFSDASAEDIGNRAEDICRVISETAPFINDLRAVDLDSLNDDGYEAAVSKIRESLEPYMDLFHELEIFSVGDEFPKCAAFYSELEEVSEQLIEIIPLFNKARSFCTRKRYSTDKIKVNLKFPTLADGWDLNKERDNKAAILRKDGKYYLAILDMKKDLSSIRTSDEDESSFEKMEYKLLPSPVKMLPKIFVKSKAAKEKYGLTDRMLECYDKGMHKSGSAFDLGFCHELIDYYKRCIAEYPGWDVFDFKFRETSDYGSMKEFNEDVAGAGYYMSLRKIPCSEVYRLLDEKSIYLFQIYNKDYSENAHGNKNMHTMYWEGLFSPQNLESPVFKLSGGAELFFRKSSIPNDAKTVHPKGSVLVPRNDVNGRRIPDSIYRELTRYFNRGDCRISDEAKSYLDKVKTKKADHDIVKDRRFTVDKMMFHVPIAMNFKAISKPNLNKKVIDGIIDDQDLKIIGIDRGERNLIYVTMVDRKGNILYQDSLNILNGYDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTTLASVGKQCGVIFYIPAAFTSKIDPTTGFADLFALSNVKNVASMREFFSKMKSVIYDKAEGKFAFTFDYLDYNVKSECGRTLWTVYTVGERFTYSRVNREYVRKVPTDIIYDALQKAGISVEGDLRDRIAESDGDTLKSIFYAFKYALDMRVENREEDYIQSPVKNASGEFFCSKNAGKSLPQDSDANGAYNIALKGILQLRMLSEQYDPNAESIRLPLITNKAWLTFMQSGMKTWKN

SEQ ID NO: 8 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 9 - protein, 1206 aa, Butyrivibrio sp.
  source 1..1206: note = NC3005
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 10 - protein, 1298 aa, Thiomicrospira sp.
  source 1..1298: note = XS5
MTKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 11 - protein, 1251 aa, Moraxella bovoculi
MLFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1222 aa, unidentified
  source 1..1222: note = Lachnospiracea bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLLKRPAATKKAGQAKKKK

SEQ ID NO: 13 - protein, 17 aa, synthetic construct
  REGION 1..17: note = Core lid domain consensus sequence or motif
  VARIANT 2..3: note = X can be any naturally occurring amino acid
  VARIANT 7..8: note = X can be any naturally occurring amino acid
  VARIANT 10..13: note = X can be any naturally occurring amino acid
  VARIANT 15..17: note = X can be any naturally occurring amino acid
LXXGFKXXRXXXXEXXX

SEQ ID NO: 14 - protein, 1228 aa, synthetic construct
  REGION 1..1228: note = Quadruple lid mutant
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFGGAGVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 15 - protein, 1220 aa, synthetic construct
  REGION 1..1220: note = Lid deletion mutant RuvC Lid deletion 1
  VARIANT 1220: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 16 - protein, 1228 aa, synthetic construct
  REGION 1..1228: note = Lid mutant pRV026004.25836.C4
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGKFSFLVSSSKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 17 - protein, 1228 aa, synthetic construct
  REGION 1..1228: note = Lid mutant pRV26184.26182 rational
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNWSCNGGLQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 18 - protein, 1228 aa, synthetic construct
  REGION 1..1228: note = Lid mutant pRV26182.25836
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGLLFWSCNGGLQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 19 - protein, 1228 aa, synthetic construct
  REGION 1..1228: note = Lid mutant pRV26010.25836. rational
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNKWRYPEAQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 20 - protein, 1228 aa, synthetic construct
  REGION 1..1228: note = Lid mutant pRV026006.25836.C6
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGIIMLKHASGCQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 21 - protein, 1228 aa, synthetic construct
  REGION 1..1228: note = Lid mutant pRV026008.25836.C8
  VARIANT 1228: note = X can be any naturally occurring amino acid
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGYWLHRQFTGRQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKX

SEQ ID NO: 22 - protein, 1227 aa, synthetic construct
  REGION 1..1227: note = pRV26180 RuvC-lid variant
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNFLVSSSKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 23 - protein, 1227 aa, synthetic construct
  REGION 1..1227: note = pRV26002 RuvC-lid mutant
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGSINKWRYPEAQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 24 - protein, 1141 aa, synthetic construct
  REGION 1..1141: note = pRV Lid1.2 Lid-deletion mutant
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 25 - protein, 1219 aa, synthetic construct
  REGION 1..1219: note = pRV Lid2.3 mutant
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYCSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 26 - protein, 1227 aa, synthetic construct
  REGION 1..1227: note = pRV Lid2.4 mutant
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGPLVRAEEAAMQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 27 - protein, 1228 aa, synthetic construct
  REGION 1..1228: note = LbCas12a-RR
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 28 - protein, 1228 aa, synthetic construct
  REGION 1..1228: note = LbCas12a-RVR
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 29 - protein, 1228 aa, synthetic construct
  REGION 1..1228: note = enhanced LbCas12a
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDRETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 30 - protein, 1228 aa, synthetic construct
  REGION 1..1228: note = LbCas12a ultra
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYLLHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 31 - protein, 1228 aa, synthetic construct
  REGION 1..1228: note = LbCas12a-RR ultra
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYLLHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 32 - protein, 1228 aa, synthetic construct
  REGION 1..1228: note = LbCas12a-RVR ultra
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYLLHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 33 - protein, 1228 aa, synthetic construct
  REGION 1..1228: note = LbCas12a D156R
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFRNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 34 - DNA, 57 bp, synthetic construct
  misc_feature 1..57: note = Top Strand Fig. 7A
  source 1..57: mol_type = other DNA
cgcgccgaggtgaagttcgagggcgacaccctggtgaaccgcatcgagctgaagggc

SEQ ID NO: 35 - DNA, 57 bp, synthetic construct
  misc_feature 1..57: note = Bottom Strand Fig. 7A
  source 1..57: mol_type = other DNA
gcgcggctccacttcaagctcccgctgtgggaccacttggcgtagctcgacttcccg

SEQ ID NO: 36 - DNA, 21 bp, synthetic construct
  misc_feature 1..21: note = SV40 Nuclear Localization Signal
  source 1..21: mol_type = other DNA
cccaagaagaagaggaaggtg

SEQ ID NO: 37 - DNA, 48 bp, synthetic construct
  misc_feature 1..48: note = Nuc Xenopus NLS sequence
  source 1..48: mol_type = other DNA
aagaggccggcggccaccaagaaggccggccaggccaagaagaagaag

SEQ ID NO: 38 - DNA, 1997 bp, synthetic construct
  misc_feature 1..1997: note = Promoter Zea mays Ubi
  source 1..1997: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 39 - DNA, 259 bp, synthetic construct
  misc_feature 1..259: note = Nos terminator Agrobacterium
  source 1..259: mol_type = other DNA
gcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttact

SEQ ID NO: 40 - DNA, 225 bp, synthetic construct
  misc_feature 1..225: note = 35S terminator
  source 1..225: mol_type = other DNA
tggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 41 - DNA, 21 bp, synthetic construct
  misc_feature 1..21: note = crRNA direct repeat LbCas12a
  source 1..21: mol_type = other DNA
taatttctactaagtgtagat

SEQ ID NO: 42 - skipped

SEQ ID NO: 43 - DNA, 516 bp, synthetic construct
  misc_feature 1..516: note = U6 promoter Oryza sativa
  source 1..516: mol_type = other DNA
tttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagctt

SEQ ID NO: 44 - DNA, 24 bp, synthetic construct
  misc_feature 1..24: note = cr-AAT-G2
  source 1..24: mol_type = other DNA
ggttcgctcgacctgatcggtgct

SEQ ID NO: 45 - DNA, 18 bp, synthetic construct
  misc_feature 1..18: note = sAAT_EZ_Fwd2 primer
  source 1..18: mol_type = other DNA
atcctcggggtaagatcc

SEQ ID NO: 46 - DNA, 20 bp, synthetic construct
  misc_feature 1..20: note = OsAAT_EZ_Rev1 primer
  source 1..20: mol_type = other DNA
gaccacattcagcaccaggg

SEQ ID NO: 47 - DNA, 720 bp, synthetic construct
  misc_feature 1..720: note = mGFP
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtttccaccctgacctacggcgtgcagtgcttcagccgctaccccgaccacatgaaacagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 48 - DNA, 678 bp, synthetic construct
  misc_feature 1..678: note = mdsRed
  source 1..678: mol_type = other DNA
atgcggtcttccaagaatgttatcaaggagttcatgaggtttaaggttcgcatggaaggaacggtcaatgggcacgagtttgaaatagaaggcgaaggagaggggaggccatacgaaggccacaataccgtaaagcttaaggtaaccaaggggggacctttgccatttgcttgggatattttgtcaccacaatttcagtatggaagcaaggtatatgtcaagcaccctgccgacataccagactataaaaagctgtcatttcctgaaggatttaaatgggaaagggtcatgaactttgaagacggtggcgtcgttactgtaacccaggattccagtttgcaggatggctgtttcatctacaaggtcaagttcattggcgtgaactttccttccgatggacctgttatgcaaaagaagacaatgggctgggaagccagcactgagcgtttgtatcctcgtgatggcgtgttgaaaggagagattcataaggctctgaagctgaaagacggtggtcattacctagttgaattcaaaagtatttacatggcaaagaagcctgtgcagctaccagggtactactatgttgactccaaactggatataacaagccacaacgaagactatacaatcgttgagcagtatgaaagaaccgagggacgccaccatctgttcctttaa

SEQ ID NO: 49 - DNA, 23 bp, synthetic construct
  misc_feature 1..23: note = cr-GFP-G6
  source 1..23: mol_type = other DNA
atgtggtcggggtagcggctgaa

SEQ ID NO: 50 - DNA, 23 bp, synthetic construct
  misc_feature 1..23: note = cr-GFP-G7
  source 1..23: mol_type = other DNA
caccctgacctacggcgtgcagt

SEQ ID NO: 51 - DNA, 23 bp, synthetic construct
  misc_feature 1..23: note = cr-dsRed-G1
  source 1..23: mol_type = other DNA
aggttcgcatggaaggaacggtc

SEQ ID NO: 52 - DNA, 4434 bp, synthetic construct
  misc_feature 1..4434: note = GFP plasmid
  source 1..4434: mol_type = other DNA
gagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtctttaacgatgggaaaccttaccctcggaccaaggtacccattaacctataaaaataggcgtcgaggccctttcgtcttccttagtcgagtgcaaaacctttcgcggtatggcatgatagcgcccggaagagagtcaattcaggggatccggggatcctctagatttaagaaggagatatacatatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcgcgtatggtcttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatgggcatggatgaactatacaaataaatgtccagacctgcaggcatgcaagctctagaggcatcaaataaaacgaaaggctcagtcgaaagactgggcctttcgttttatctgttgtttgtcggtgaacgctctcctgagtaggacaaatccgccgccctagacctaggcaaaaaacccctcaagacccgtttagaggccccaaggggttatgctagttattgctcagcggaatctacacttagtagaaattatttaatctttgaaacagggtaaggtttcccatcgtatctacacttagtagaaattatttaatctttgaaacgctagcattatacctaggactgagctagctgtcaaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcgccgtcgcaagctccggcaaatcgctgaatattccttttgtctccgaccatcaggcacctgagtcgctgtctttttcgtcacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagc

SEQ ID NO: 53 - DNA, 4518 bp, synthetic construct
  misc_feature 1..4518: note = RFP plasmid
  source 1..4518: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacggggcctgccaccatacccacgccgaaacaagcggatcctttaacgatgggaaaccttaccctcggaccaatacaactgttggtccgattccccaggttcagtggagtaaagacgtcgctcatgagcccgaagtggcgcgacccgaaaagtgccacttgcaaaaaaaccccgccgaagcggggaggtataggcgcctcgcatcaatgataacacatcattatactagtttaagcaccggtggagtgacgaccttcagcacgttcgtactgttcaacgatggtgtaatcttcgttgtgggaggtgatgtccagtttgatgtcggttttgtaagcacccggcagctgaaccggttttttagccatgtaggtggttttaacttcagcgtcgtagtgaccaccgtctttcagtttcagacgcattttgatttcacctttcagagcaccatcttccgggtacatacgttcggtggaagcttcccaacccatggtttttttctgcataaccggaccgtcggacgggaagttggtaccacgcagtttaactttgtagatgaactcaccgtcttgcagggaggagtcctgggtaacggtaacaacaccaccatcttcgaagttcataacacgttcccatttgaaaccttccgggaaggacagtttcaggtagtccgggatgtcagccgggtgtttaacgtaagctttggaaccgtactggaactgcggggacaggatgtcccaagcgaacggcagcgggccacctttggtaactttcagtttagcggtctgtgtaccttcgtacggacgaccttcaccttcaccttcgatttcgaactcgtgaccgttaacggaaccttccatacgaactttgaaacgcatgaactctttgataacatcttcggaggaagccatatgtatatctccttcttatagttaaacaaaattatttctagagggaaaccgttgtggtctccccacacattgtctcatgagcggatacatatttgaacttgaaaaaaccccgccgaagcggggtaccgaattacaacttatatcgtatgggagcccgatcttccccatcggtgatgtcggcgatataggcgccagcaaccgcacctgtggcgccggtgatgccggccacgatgcgtccggcgtagaggatctgctcatgtttgacagcttatcatcgatgcatttgacagctagctcagtcctaggtataatgctagcgtttcaaagattaaataatttctactaagtgtagatctccactgaacctggggaatgtttcaaagattaaataatttctactaagtgtagattttttttgccggcttatcggtcagtttcacctgatttacgtaaaaacccgcttcggcgggtttttgcttttggaggggcagaaagatgaatgactgtccacgacgctatacccaaaagaaaaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttc

SEQ ID NO: 54 - DNA, 7742 bp, synthetic construct
  misc_feature 1..7742: note = Cas wild-type plasmid
  source 1..7742: mol_type = other DNA
ttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttggggcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcatctgacagctagctcagtcctaggtataatgctagcgagctcaggaggacatcatgagcaaactggaaaaatttacgaattgttatagcctgtccaagaccctgcgtttcaaagccatccccgttggcaaaacccaggagaatattgataataaacgtctgctggttgaggatgaaaaaagagcagaagactataagggagtcaaaaaactgctggatcggtactacctgagctttataaatgacgtgctgcatagcattaaactgaaaaatctgaataactatattagtctgttccgcaagaaaacccgaacagagaaagaaaataaagagctggaaaacctggagatcaatctgcgtaaagagatcgcaaaagcttttaaaggaaatgaaggttataaaagcctgttcaaaaaagacattattgaaaccatcctgccggaatttctggatgataaagacgagatagcgctcgtgaacagcttcaacgggttcacgaccgccttcacgggctttttcgataacagggaaaatatgttttcagaggaagccaaaagcacctcgatagcgttccgttgcattaatgaaaatttgacaagatatatcagcaacatggatattttcgagaaagttgatgcgatctttgacaaacatgaagtgcaggagattaaggaaaaaattctgaacagcgattatgatgttgaggattttttcgagggggaattttttaactttgtactgacacaggaaggtatagatgtgtataatgctattatcggcgggttcgttaccgaatccggcgagaaaattaagggtctgaatgagtacatcaatctgtataaccaaaagaccaaacagaaactgccaaaattcaaaccgctgtacaagcaagtcctgagcgatcgggaaagcttgagcttttacggtgaaggttataccagcgacgaggaggtactggaggtctttcgcaataccctgaacaagaacagcgaaattttcagctccattaaaaagctggagaaactgtttaagaattttgacgagtacagcagcgcaggtatttttgtgaagaacggacctgccataagcaccattagcaaggatatttttggagagtggaatgttatccgtgataaatggaacgcggaatatgatgacatacacctgaaaaagaaggctgtggtaactgagaaatatgaagacgatcgccgcaaaagctttaaaaaaatcggcagctttagcctggagcagctgcaggaatatgcggacgccgacctgagcgtggtcgagaaactgaaggaaattattatccaaaaagtggatgagatttacaaggtatatggtagcagcgaaaaactgtttgatgcggacttcgttctggaaaaaagcctgaaaaaaaatgatgctgttgttgcgatcatgaaagacctgctcgatagcgttaagagctttgaaaattacattaaagcattctttggcgagggcaaagaaacaaacagagacgaaagcttttatggcgacttcgtcctggcttatgacatcctgttgaaggtagatcatatatatgatgcaattcgtaattacgtaacccaaaagccgtacagcaaagataagttcaaactgtatttccagaacccgcagtttatgggtggctgggacaaagacaaggagacagactatcgcgccactattctgcgttacggcagcaagtactatctcgccatcatggacaaaaaatatgcaaagtgtctgcagaaaatcgataaagacgacgtgaacggaaattacgaaaagattaattataagctgctgccagggcccaacaagatgttaccgaaagtatttttttccaaaaaatggatggcatactataacccgagcgaggatatacagaagatttacaaaaatgggaccttcaaaaagggggatatgttcaatctgaatgactgccacaaactgatcgatttttttaaagatagcatcagccgttatcctaaatggtcaaacgcgtatgattttaatttctccgaaacggagaaatataaagacattgctggtttctatcgcgaagtcgaagaacagggttataaagttagctttgaatcggccagcaagaaagaggttgataaactggtggaggagggtaagctgtatatgtttcagatttataacaaagactttagcgacaaaagccacggtactcctaatctgcatacgatgtactttaaactgctgtttgatgagaataaccacggccaaatccgtctctccggtggagcagaactttttatgcggcgtgcgagcctaaaaaaggaagaactggtggtgcatcccgccaacagcccgattgctaacaaaaatccagataatcctaagaagaccaccacactgtcgtacgatgtctataaggataaacgtttctcggaagaccagtatgaattgcatataccgatagcaattaataaatgcccaaaaaacattttcaaaatcaacactgaagttcgtgtgctgctgaaacatgatgataatccgtatgtgatcggaattgaccgtggggagagaaatctgctgtatattgtagtcgttgatggcaagggcaacatcgttgagcagtatagcctgaatgaaataattaataattttaacggtatacgtattaaaaccgactatcatagcctgctggataaaaaggagaaagagcgttttgaggcacgccaaaattggacgagcatcgaaaacatcaaggaactgaaggcaggatatatcagccaagtagtccataaaatctgtgaactggtggagaagtacgacgctgtcattgccctggaagacctcaatagcggctttaaaaacagccgggtgaaggtggagaaacaggtataccaaaagtttgaaaagatgctcattgataagctgaactatatggttgataaaaagagcaacccgtgcgccactggcggtgcactgaaagggtaccaaattaccaataaatttgaaagctttaaaagcatgagcacgcagaatgggtttattttttatataccagcatggctgacgagcaagattgaccccagcactggttttgtcaatctgctgaaaaccaaatacacaagcattgcggatagcaaaaaatttatttcgagcttcgaccgtattatgtatgttccggaggaagatctgtttgaatttgccctggattataaaaacttcagccgcaccgatgcagattatatcaaaaaatggaagctgtacagttatggtaatcgtatacgtatcttccgtaatccgaagaaaaacaatgtgttcgattgggaagaggtctgtctgaccagcgcgtataaagaactgttcaacaagtacggaataaattatcagcaaggtgacattcgcgcactgctgtgtgaacagtcagataaagcattttatagcagctttatggcgctgatgagcctgatgctccagatgcgcaacagcataaccggtcgcacagatgttgactttctgatcagccctgtgaagaatagcgacggcatcttctacgattccaggaactatgaagcacaggaaaacgctattctgcctaaaaatgccgatgccaacggcgcctataatattgcacggaaggttctgtgggcgattggacagttcaagaaagcggaagatgagaagctggataaggtaaaaattgctattagcaataaggaatggctggagtacgcacagacatcggttaaataaaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttat

SEQ ID NO: 55 - DNA, 7742 bp, synthetic construct; identical to SEQ ID NO: 54
  misc_feature 1..7742: note = Cas dead plasmid
  source 1..7742: mol_type = other DNA

SEQ ID NO: 56 - protein, 1220 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYESSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 57 - RNA, 23 nt, synthetic construct
  source 1..23: mol_type = other RNA; note = protospacer of LbCas12a gRNA targeting the Brassica napus FAD2 gene (LOC106452409)
tcctacctcatctgggacatcat

SEQ ID NO: 58 - skipped

SEQ ID NO: 59 - RNA, 23 nt, synthetic construct
  source 1..23: mol_type = other RNA; note = cr-DEP1-G3 of Fig. 13 and Fig. 14, protospacer of LbCas12a gRNA targeting the rice OsDEP1 gene (LOC_Os09g26999)
atacttgacataagctcttcaaa

SEQ ID NO: 60 - RNA, 23 nt, synthetic construct
  source 1..23: mol_type = other RNA; note = cr-DEP1-G4 of Fig. 13, protospacer of LbCas12a gRNA targeting the rice OsDEP1 gene (LOC_Os09g26999)
agtttttagaaatttgaagagct

SEQ ID NO: 61 - RNA, 23 nt, synthetic construct
  source 1..23: mol_type = other RNA; note = protospacer of LbCas12a gRNA targeting the rice OsDEP1 gene (LOC_Os09g26999)
ttttccattgtggtgcatggtca

SEQ ID NO: 62 - RNA, 23 nt, synthetic construct
  source 1..23: mol_type = other RNA; note = cr-DEP1-G6 of Fig. 13, protospacer of LbCas12a gRNA targeting the rice OsDEP1 gene (LOC_Os09g26999)
gtttttattttccattgtggtgc

SEQ ID NO: 63 - RNA, 23 nt, synthetic construct
  source 1..23: mol_type = other RNA; note = cr-DEP1-G7, protospacer of LbCas12a gRNA targeting the rice OsDEP1 gene (LOC_Os09g26999)
taattcagtactgcaggctgagt

SEQ ID NO: 64 - RNA, 23 nt, synthetic construct
  source 1..23: mol_type = other RNA; note = cr-DEP1-G8 of Fig. 13, protospacer of LbCas12a gRNA targeting the rice OsDEP1 gene (LOC_Os09g26999)
tcatttctctcatgttaagtgca

SEQ ID NO: 65 - skipped

SEQ ID NO: 66 - RNA, 23 nt, synthetic construct
  source 1..23: mol_type = other RNA; note = protospacer of LbCas12a gRNA targeting the rice OsDEP1 gene (LOC_Os09g26999)
tacctgttgaagtaaaatagact

SEQ ID NO: 67-69 - skipped (3 entries)

SEQ ID NO: 70 - RNA, 23 nt, synthetic construct
  source 1..23: mol_type = other RNA; note = cr-DEP1-G14 of Fig. 13, protospacer of LbCas12a gRNA targeting the rice OsDEP1 gene (LOC_Os09g26999)
taggatgagcttcacttccttga

SEQ ID NO: 71 - RNA, 23 nt, synthetic construct
  source 1..23: mol_type = other RNA; note = cr-DEP1-G15 of Fig. 13, protospacer of LbCas12a gRNA targeting the rice OsDEP1 gene (LOC_Os09g26999)
cattgtggtgcatggtcactcag

SEQ ID NO: 72 - RNA, 23 nt, synthetic construct
  source 1..23: mol_type = other RNA; note = cr-DEP1-G16 of Fig. 13, protospacer of LbCas12a gRNA targeting the rice OsDEP1 gene (LOC_Os09g26999)
taaaaacttaaaaacaaaatatc

SEQ ID NO: 73 - RNA, 23 nt, synthetic construct
  source 1..23: mol_type = other RNA; note = cr-DEP1-G17 of Fig. 13 and Fig. 14, protospacer of LbCas12a gRNA targeting the rice OsDEP1 gene (LOC_Os09g26999)
tcgttctggatgcattaaagagt

SEQ ID NO: 74 - RNA, 20 nt, synthetic construct
  source 1..20: mol_type = other RNA; note = protospacer of LbCas12a gRNA targeting the human DYRK1A gene
acttgggtgccctaggaagc

SEQ ID NO: 75 - RNA, 20 nt, synthetic construct
  source 1..20: mol_type = other RNA; note = protospacer of LbCas12a gRNA targeting the human DYRK1A gene
ctaccgctgaagtccgtgtc

SEQ ID NO: 76 - RNA, 20 nt, synthetic construct
  source 1..20: mol_type = other RNA; note = protospacer of LbCas12a gRNA targeting the human DYRK1A gene
agggggtagcatttctctgt

SEQ ID NO: 77 - RNA, 20 nt, synthetic construct
  source 1..20: mol_type = other RNA; note = protospacer of LbCas12a gRNA targeting the human DYRK1A gene
gaatgaaggggagggtgtga

SEQ ID NO: 78 - RNA, 20 nt, synthetic construct
  source 1..20: mol_type = other RNA; note = protospacer of LbCas12a gRNA targeting the human DYRK1A gene
gcgctgtcaagaaccagaat

SEQ ID NO: 79 - RNA, 20 nt, synthetic construct
  source 1..20: mol_type = other RNA; note = protospacer of LbCas12a gRNA targeting the human DYRK1A gene
tcccaggagtgacaagataa

SEQ ID NO: 80 - DNA, 3663 bp, synthetic construct
  source 1..3663: mol_type = other DNA; note = LbCas12a RuvC L-del1, codon optimized for Bacillus subtilis
atgtctaaattggagaaatttacaaactgctactctctgtcgaagactctacggtttaaagctatcccggtaggcaagacccaagaaaatattgataataaacgtttactggtcgaagatgaaaaacgagctgaagactataaaggcgtgaaaaagcttctggatcggtactatctgagctttatcaacgacgtgcttcacagcattaaattgaaaaatctcaacaattacatcagtttgtttcgaaaaaagacacgtacagagaaggaaaataaagagctggaaaatcttgaaataaacttacgaaaagaaatcgctaaggcatttaaaggaaacgagggatataaaagcctatttaaaaaggacatcattgaaactattctcccggaattcttagatgacaaggacgaaatcgctctggtgaactcattcaatggatttaccacagcttttacgggatttttcgataaccgagaaaatatgttctctgaagaggccaagtctacctccattgcatttaggtgtattaacgaaaacttgacgcggtatatctccaacatggatatctttgagaaggtggacgcgatatttgataagcacgaagtccaggaaattaaagaaaaaattctcaattcagattacgatgttgaggattttttcgaaggagaatttttcaactttgtgcttacccaagaaggtatagatgtttataatgcaattataggcggatttgtaactgaatcaggcgagaaaattaaaggattgaatgaatacataaatctgtataatcagaaaacaaaacaaaagctgcctaagtttaagccgctgtataagcaggtcttatctgacagggaaagtttatctttctacggcgagggctatacaagcgatgaagaggtcctggaagtctttagaaacactttgaacaaaaattcagaaattttttctagcattaaaaagttagaaaaactttttaaaaattttgatgagtacagttcggccggcattttcgtgaaaaatggacctgctatcagcacgatcagcaaagatatttttggagagtggaatgttatcagagataagtggaatgccgagtatgatgacatccatctgaagaaaaaggctgtcgttacggagaagtacgaagacgatcgcagaaagtcctttaagaaaattggttccttcagtttagagcaacttcaagaatatgccgacgctgatcttagcgttgtggaaaaacttaaggaaattatcattcagaaggtagatgaaatttataaggtatacggctcaagcgagaaattattcgatgccgattttgtactcgagaaatctttgaaaaagaacgatgcggtcgtggcgataatgaaagatctcttagacagcgttaaaagcttcgaaaactatattaaggcttttttcggcgagggcaaagaaactaaccgtgacgaatcattctatggtgactttgtccttgcctatgacattctgcttaaagtggatcatatttacgacgccatacggaactatgtaacccagaagccctatagcaaagataaatttaaactgtacttccaaaacccgcagtttatgggtggctgggacaaagacaaagaaacagattatagagcaacgattctacgctatggctcgaaatattacttggcaatcatggataaaaagtacgcgaaatgcttgcagaaaattgacaaagatgacgttaacggcaattatgagaagattaactacaagttgctgccaggcccgaataagatgcttcctaaggtcttcttttccaaaaagtggatggcgtattacaatccttcagaagacatccaaaagatctacaaaaatggtacttttaaaaagggtgatatgtttaatttaaacgattgtcataaactgatagattttttcaaagactccatttcaagatatccaaaatggagcaatgcctatgacttcaattttagcgaaacggagaagtataaagatatagcgggtttctatcgtgaagtagaagagcagggctataaagtgagctttgaatccgccagcaaaaaggaggttgacaaactcgtggaagagggcaaactttacatgttccagatttataataaagatttctcggataaatcacatggaacacctaatcttcatacaatgtatttcaagcttctctttgatgagaataaccatggccaaatcagactttcaggcggagcggaattgtttatgcgcagggcgtcactcaaaaaggaagagctcgttgtgcacccggcgaattccccgattgcgaacaaaaacccggacaacccaaagaaaacaacgacgttatcgtacgatgtttataaagataaacgcttttctgaagatcagtatgaattgcatatcccaatcgcaattaataaatgccctaagaatatttttaaaattaacacagaagtcagagtgttgctcaaacatgatgacaatccatacgtgatcggcatcgatcgcggagagagaaatctcttatacattgtggtagtcgacggaaaaggtaacattgtcgaacaatattccttgaacgaaataatcaataacttcaatggcatccggattaaaacagattatcacagtcttctggataagaaagaaaaagaacgctttgaagcaagacagaattggacgtctattgaaaatatcaaagaattgaaggcgggctatattagccaagtggttcataaaatttgtgaacttgtcgaaaaatatgacgctgtaattgccttagaggacctgaattcatattgttcaagcgtctaccaaaagtttgaaaaaatgttaatcgacaaactaaactacatggtcgataaaaagagcaatccgtgcgctacgggaggcgcgctaaagggctatcaaattaccaacaaatttgagtcttttaagtccatgtccacgcaaaatggattcatcttttatattccggcctggctgacatctaagattgatccgtcgaccggcttcgttaacttactgaaaacaaagtatacatctatcgcagacagtaaaaagtttatttcttcgtttgataggatcatgtatgtacctgaggaagacctgttcgaattcgcattagattataaaaatttctcgcgcacagatgcagattacatcaagaaatggaaactttacagctatggtaacagaattcgtatcttccgcaatcccaaaaagaacaatgtctttgactgggaggaagtgtgcctgacgagtgcctataaggaattgtttaataagtatggaatcaattaccagcaaggtgatatccgggcacttctgtgtgaacagtcagataaagcgttctactcatcttttatggcacttatgtctttaatgttacaaatgagaaactcaattacgggacggacagatgtagattttcttatatctccggttaaaaatagcgatggcattttctacgattcacgtaactatgaagctcaagagaatgcaatactgcctaaaaacgctgacgcgaatggagcttataacatcgcacgtaaagttctgtgggctatcggccagtttaaaaaggcggaagatgaaaaattagacaaagttaaaattgcaatttcaaataaagagtggcttgaatacgcacagactagtgttaaacactaa

SEQ ID NO: 81 - DNA, 3663 bp, synthetic construct
  source 1..3663: mol_type = other DNA; note = LbCas12a RuvC L-del1, codon optimized for Rhodococcus spp.
atgagcaagctcgaaaagttcaccaactgctattccctgagcaagacgctgcgcttcaaggccatccccgtgggtaagacgcaggagaacatcgataataagcgcctgctcgtggaagatgagaaacgtgccgaggactacaagggcgtgaagaaactgctcgaccgctactatctgtccttcatcaacgacgtgctccactccatcaagctcaagaacctcaacaattacatctcgctgttccgcaaaaagacccgtaccgagaaggagaacaaggagctcgagaacctcgagatcaacctgcggaaggagatcgcgaaggcattcaagggcaacgaggggtacaagtccctgttcaagaaagacatcattgagaccatcctccccgagttcctcgacgataaggacgagatcgccctggtgaactcgttcaacggattcacgaccgccttcaccggcttctttgacaaccgtgagaacatgttctcggaggaagccaagtcgacgtcaatcgctttccgctgcatcaacgagaatctgacccgctacatcagcaacatggacatctttgagaaggtcgacgcgatcttcgacaagcacgaggtgcaggagatcaaagagaagatcctcaactcggactacgacgtcgaggacttctttgaaggcgaattctttaacttcgtgctcacccaggagggcatcgacgtctacaacgccatcattggtggcttcgtgaccgagagcggcgaaaagatcaagggcctgaacgaatacatcaacttgtacaaccagaagacgaagcagaagctcccgaagttcaagccgctgtacaagcaggtgctctcggaccgggagtcgctcagcttctacggtgagggctacacgtcggacgaagaggtgctggaggtcttccgtaacaccctcaacaagaactcggagatcttctcgagcattaagaaactggagaagctcttcaagaacttcgacgagtattcgagtgcgggcatcttcgtcaagaacggaccggccatctccaccatctctaaggacatcttcggtgagtggaatgtgatccgggacaagtggaacgcggaatacgacgatatccacctcaagaaaaaagcggtcgtgaccgagaagtacgaagacgatcgccgaaagtccttcaagaaaatcggttcgttcagcctcgagcagctccaggaatacgcggacgcggatctcagcgtggtcgaaaagctgaaggagatcattattcaaaaggtcgacgagatctacaaggtctacggcagctcggagaagctgttcgacgcagacttcgtcctcgagaagtccctgaagaaaaacgacgccgtcgtggccatcatgaaggacctcctggactcggtcaagagcttcgagaattacatcaaggcgttctttggtgagggtaaggaaaccaaccgcgacgagtcattctacggtgacttcgtcctggcgtacgatatcctgctcaaagtggatcacatctatgacgccatccggaactacgtcacccagaagccgtattccaaagataagttcaagctctacttccagaacccgcagttcatgggcggttgggacaaggacaaggaaacggactaccgcgcgaccatcctgcggtatgggtcgaagtactatctcgccatcatggacaagaaatacgcgaagtgcctgcagaagatcgataaggacgatgtcaacggcaactacgaaaagatcaattacaagctcctgcccggaccgaacaagatgctgcctaaggtcttcttttcgaagaaatggatggcgtactataacccaagcgaagacatccagaagatctataagaacgggacgttcaagaaaggtgacatgttcaacctgaacgattgccacaaactcatcgacttctttaaggacagcatctcgcgctacccgaagtggtcgaacgcgtatgacttcaacttctccgaaactgagaagtacaaggacatcgccgggttctaccgggaagtggaggaacagggctacaaggtttccttcgaatccgcctccaagaaagaggtcgataagctcgtcgaggaaggcaagttgtacatgttccagatctacaacaaggacttctccgacaagtcccacggcacgccgaatctgcatacgatgtattttaagctgctcttcgacgaaaacaatcatggccagatccgcctgagcggcggagccgagctcttcatgcgtcgcgcctccctgaagaaagaggaactcgtggtccacccggccaactccccgatcgccaacaagaaccccgacaatcccaagaaaaccacgacgctcagctacgacgtttataaggacaagcgcttctcggaggaccagtacgagctgcacataccgatcgccattaacaaatgccccaagaatatcttcaagatcaacaccgaggtgcgggtgctcctgaagcacgacgataacccgtatgtcatcggcatcgaccgcggtgagcgcaaccttctgtacatcgtcgtagtcgacggcaagggaaacatcgtggaacagtacagcctcaacgaaatcattaacaatttcaacgggatccgcatcaaaaccgactaccactccctgctcgacaaaaaggaaaaggaacgcttcgaagcccgccagaactggacctcgatcgagaacatcaaggagctgaaggcggggtacatctcccaggtcgtgcacaagatctgcgagctcgtggagaagtacgatgccgtcatcgcgctggaggacttgaacagctactgttccagtgtgtaccagaagttcgagaagatgctcattgacaagctcaactacatggtcgacaagaaatccaacccctgcgcgacgggcggagcgctcaagggctaccaaatcaccaacaagttcgaatcgttcaagtccatgtcgacccagaacggattcatcttctacatcccggcctggctcacgtcgaaaatcgacccctcgaccggattcgtcaatctgctcaagaccaagtacacctcgatcgccgactccaagaaattcatctcgtccttcgatcggatcatgtacgtccccgaagaggacctcttcgagttcgctttggactacaagaacttctcgcgaaccgatgccgactacatcaagaaatggaagctctactcgtacggtaaccgcatcaggatcttccggaacccgaagaaaaacaatgtcttcgactgggaggaagtctgcctgacgagtgcgtacaaagagctcttcaacaagtacggcatcaactaccagcaaggcgacatccgggccctcctgtgcgagcagagcgacaaggcattctactcctcgttcatggcgctgatgtcgttgatgttgcagatgcgcaactcgatcacaggtcgcaccgatgtcgacttcctgatctcgccggtcaagaactcggacgggatcttctacgactcgcgaaactacgaagcgcaggagaacgcgatcctgcccaagaacgcggacgcaaacggcgcctacaatatcgcccggaaggtcctctgggctatcgggcaattcaagaaagccgaggacgagaagctggataaggtcaaaatcgcgatctccaacaaggagtggctggagtacgcacagacatcggtcaaacactga

SEQ ID NO: 82 - DNA, 3663 bp, synthetic construct
  source 1..3663: mol_type = other DNA; note = LbCas12a RuvC L-del1, codon optimized for Yarrowia lipolytica
atgtcgaagttggagaagtttacaaactgttactctctttcgaagacccttagatttaaagctatcccggtgggcaagacacaggaaaatatcgacaacaagcgactgctcgttgaggacgagaagcgggcagaggactacaagggagtcaaaaagctccttgatcgctactatctgtcttttatcaatgacgtgctgcactctattaaacttaagaacctgaacaattatatcagtttgttccgaaagaaaacgcggactgagaaggagaacaaggaattggagaacttggagatcaacttaagaaaggagatcgctaaggctttcaaggggaacgagggttacaagtcgctctttaagaaagacatcattgagacaatcctgcccgaattcctggatgacaaggacgagatcgccctagtcaactccttcaatggcttcaccacggcgttcactggcttctttgacaaccgagaaaacatgttcagtgaagaggctaaatctacctccattgcttttcggtgcatcaacgaaaacctcactcgctatataagcaacatggacatttttgagaaggtcgacgccatcttcgataagcacgaggtccaggagatcaaggagaaaatccttaactcggattatgatgttgaagacttctttgagggcgaatttttcaacttcgtgctgacccaggagggaatcgacgtgtacaatgcgattatcggcggattcgtaaccgagtcaggtgagaaaatcaaaggcctgaatgagtacattaacctctacaaccagaaaacgaagcagaagcttcccaagttcaagcccctatacaagcaagtgctgagcgacagagagagcctgagcttttacggagaaggctacacctccgacgaggaagttctggaagtgttcagaaatactctgaacaagaactcggagatcttctcaagtatcaagaaactcgagaagctgtttaagaactttgatgaatactcctcggccggaatctttgttaagaacggtcctgctatctccaccatctcaaaggacattttcggagaatggaatgttattcgtgacaaatggaacgccgagtacgacgatattcacctcaagaaaaaggccgtggttaccgagaaatatgaggatgaccggcgaaagagcttcaagaaaattggaagtttttcgctcgagcagctacaagagtacgccgatgccgacctctccgttgtagagaagctgaaggagattatcattcagaaggtggacgagatctacaaggtgtacggctcctctgagaagctgttcgatgccgactttgtccttgagaagtccctgaaaaagaacgatgcagtcgtagccatcatgaaggacctgttggactctgtgaagtcgtttgaaaattacattaaggctttctttggagaaggaaaggaaactaaccgggacgagtccttctacggcgatttcgtcctggcatacgacatcttgcttaaggttgatcacatttatgacgccattcgtaactacgtgacccaaaagccctactccaaggacaagttcaagctgtatttccagaacccgcagttcatgggcggttgggataaggacaaagagactgactaccgagccacaattttgcgctacgggtcgaagtattacctggctatcatggacaagaaatacgcgaagtgtctccagaagatagacaaggacgatgtgaacggcaactacgagaaaatcaactacaagctgctccctggtcctaacaagatgctccctaaggtgttcttttcgaagaaatggatggcctattacaacccttcggaggacattcagaagatctacaagaatggaacattcaagaaaggcgacatgttcaacctcaacgattgtcataaattgatcgatttctttaaggatagcatctctcgataccccaaatggtccaatgcctacgatttcaacttttccgagaccgagaagtacaaggatattgccggtttctaccgagaggttgaggaacagggatacaaagtgtcctttgagtctgcttcgaagaaagaggttgataaactcgtcgaagagggcaagctctacatgttccagatctacaacaaagacttttccgacaaatcacacggaacccctaacctacatacgatgtattttaagttgctgtttgatgagaacaatcacggtcagattcgtctgtcgggcggtgctgagctgtttatgcgaagagcttctctcaagaaagaggaactcgttgtgcacccagcaaactctcccattgcgaataaaaatcccgacaaccctaagaaaaccactacattgtcatacgatgtctataaggataagcggttctctgaagatcagtatgagctgcacattccaattgcaatcaacaagtgtccaaagaacatcttcaagattaacacggaggtcagggtgctcctgaagcatgacgataacccctacgtcattggtattgacagaggcgagcgaaaccttctctatattgtcgtggtagacggaaagggaaacatagtcgaacagtactccctaaacgaaatcattaacaatttcaacggtattcgaattaagacggactatcatagccttctggacaaaaaggagaaggagcgatttgaggcccgacagaactggacctctattgaaaacataaaggagctgaaggccgggtacatttcccaggtcgttcataagatctgcgagctggttgaaaagtacgacgctgtcatcgcgctggaggaccttaacagctactgctcgtccgtgtaccagaaatttgaaaagatgttgattgacaaactgaactatatggtcgacaagaaaagtaacccctgcgccaccggaggtgctctgaaggggtaccagattaccaacaagtttgagtcattcaagagcatgagtacccaaaatggctttattttttacatccccgcatggctgacctctaaaatcgatccatctactggatttgtcaacctcctgaaaactaagtacacttctattgccgattctaaaaagtttatctctagcttcgatagaatcatgtacgtgcccgaggaagatctgttcgagtttgctctggactacaagaacttctcaagaaccgatgctgactacattaagaaatggaagctctactcgtatggtaatcgtatccgaatcttccgaaaccctaagaaaaacaatgtgttcgactgggaggaagtttgcctaacttctgcctataaagaacttttcaacaagtacggaatcaactaccagcaaggtgatattcgagccctcctgtgtgagcagtccgacaaggccttttactcgtcttttatggcactgatgtccctcatgcttcaaatgcgaaactccatcactggccgaacagatgtcgacttcctgatttctcctgtgaagaactctgatggtatcttttacgacagtcgcaattacgaagctcaggagaatgccattctgcccaaaaatgccgacgctaatggagcttacaacattgctcgtaaggtgctctgggcgattggccaattcaagaaagccgaagacgaaaagttggacaaggtcaagatcgccatttctaataaggagtggcttgaatatgcacagacttccgtgaagcattaa

SEQ ID NO: 83 - DNA, 3663 bp, synthetic construct
  source 1..3663: mol_type = other DNA; note = LbCas12a RuvC L-del1, codon optimized for Escherichia coli K12
atgagcaaattggaaaagttcactaattgttactcgctgagtaaaaccctgcgctttaaagccatccctgtcggaaaaacccaggaaaacatcgataacaagcgcctgctcgtcgaggatgagaaaagagcagaagattacaagggcgtgaaaaagctgttagaccgttactatttgagctttattaacgatgtgttacatagcattaagctgaaaaatttgaataactatatcagcctgttccggaaaaagacccgcacagaaaaagagaataaagagctggaaaacttggaaattaacctgcgcaaagaaatcgccaaagcatttaaagggaacgaaggatataaatccctgttcaaaaaggatattatcgaaactatcctgccggaattcctggatgacaaagacgaaatcgccctggtcaattccttcaacggctttactaccgcgtttacaggcttctttgataaccgtgaaaacatgttcagtgaagaggccaaatccacgagtatcgcgttccgttgcatcaacgaaaatctgacccgctatattagcaacatggatatcttcgaaaaagtcgatgccattttcgataaacacgaagtacaagagattaaagagaaaattctgaactccgattacgacgtcgaggacttctttgaaggggagtttttcaattttgtactgacccaagagggcattgacgtgtataatgcaattatcggtggctttgtaaccgagtcgggcgaaaaaatcaaagggctgaatgaatatattaatctgtataaccaaaaaaccaaacagaagcttccaaaattcaaaccactttataaacaggtcctgtctgatcgggaatctctgtctttttacggcgaaggttataccagcgacgaggaagtgctcgaggtctttcgtaataccctgaacaaaaattctgagatttttagcagtataaaaaagctggaaaaactgttcaaaaactttgatgaatactcatctgcgggcattttcgtgaagaatggtccggctatttctacgatttcgaaagatatcttcggtgagtggaacgtgattcgcgataaatggaacgctgaatatgatgacattcacctgaaaaagaaggcggttgtgacggaaaaatacgaagatgaccgtcgcaaatcatttaaaaagatcggcagttttagcttagaacagctgcaggaatacgctgatgccgatctgtcggtcgtggaaaaattgaaagaaataatcattcagaaagttgatgaaatttacaaagtttacggtagttcggaaaaactgtttgacgcagatttcgtcctggagaagtcgctgaaaaagaacgacgccgtggtagcaataatgaaagacctattggactcggttaaaagctttgaaaattatattaaagcctttttcggcgagggaaaggagaccaatcgtgatgagtctttctatggggattttgttttagcgtacgatatcctgttaaaggttgatcatatttacgatgcgattcgtaattatgttacccagaaaccgtatagcaaggataaatttaaactatacttccagaacccgcaatttatgggtggctgggataaagacaaggagaccgactatcgtgcgaccattctgcggtatggctctaagtactatcttgcaatcatggacaaaaagtatgcgaaatgtctgcagaagattgataaagacgatgtgaacggcaattacgaaaaaattaactataagttgctgcccgggccgaataaaatgctcccgaaagtttttttctcgaagaaatggatggcatattacaacccgagcgaagatatccagaaaatctataagaatggtactttcaaaaagggagatatgtttaacttaaacgattgccacaaattaatcgactttttcaaagacagcatttcccggtatccaaaatggtccaacgcatacgatttcaatttctcggaaacggaaaaatataaagacattgcgggattttatcgtgaagttgaggaacaaggctacaaagtgtcattcgaatcggcgtccaaaaaggaggttgataagctggttgaggaaggcaaactttacatgtttcagatctacaacaaggatttttccgacaaaagccatggtaccccgaacttacacacgatgtacttcaagctgctctttgatgaaaacaatcacgggcagattaggttgagcgggggcgcggagctcttcatgcgccgtgctagcctgaaaaaggaagagctggtcgtgcatccagcaaattcaccgatagctaacaaaaatccggataatcctaaaaagacgactaccctgagctatgacgtttacaaagataaacgcttttcagaagaccagtatgaactccatattcccatcgccattaataaatgcccgaaaaatatttttaaaatcaacacagaagtgcgtgtgctgcttaagcatgatgacaacccgtatgttatcggtattgaccgtggtgaacgaaacctgttatatatcgtagtggtagacggcaaaggaaacatcgtggaacagtatagtcttaacgaaattatcaataactttaatggcatccgcatcaagacggactaccattctttgctggataaaaaggagaaagagagattcgaagcacgtcaaaattggacaagcattgaaaatatcaaagaactcaaagccggttatatttcccaagtcgtgcataaaatctgcgaactggtagagaaatatgatgccgtgatcgcattggaagatttgaattcctattgttcaagcgtgtaccagaaattcgaaaaaatgttaatcgacaaactcaactatatggtggataaaaagagtaacccatgtgccactggtggcgcgttaaagggctaccaaatcacgaataaattcgaatcctttaagagtatgtctacacaaaacggctttattttctatatcccggcgtggttaacgagcaagattgatcctagcactggcttcgttaatttactgaaaacaaagtatacctcaatcgccgattcgaaaaagtttatttcgagctttgaccgcattatgtacgttccggaagaggatctgtttgaatttgctttagactataaaaacttttctcgcacggatgccgactatatcaaaaagtggaaactgtattcatacggtaaccgcatccgcatttttcgtaaccctaaaaagaacaatgtgtttgattgggaagaggtgtgccttacctctgcgtataaggaattatttaacaaatatggtattaattaccagcaaggcgacatacgcgcgctgctctgcgaacagagtgataaagctttctattcaagctttatggcccttatgagccttatgctccagatgcgtaactccattaccggccgtacggatgtagactttctgattagccccgtcaaaaacagtgacggtatcttttatgattcacgaaattacgaggcacaggaaaacgcgatcctgccgaaaaacgcggatgcgaatggcgcctacaacattgcgcgtaaggttttgtgggcgattggccaattcaaaaaggcggaagatgagaaattggataaagtcaaaatagctatcagtaataaagaatggcttgagtatgctcagacgagcgtaaaacactaa

SEQ ID NO: 84 - DNA, 3663 bp, synthetic construct
  source 1..3663: mol_type = other DNA; note = LbCas12a RuvC L-del1, codon optimized for Saccharomyces cerevisiae
atgtctaagctggagaagtttaccaactgttactcgttgagcaagactctgcgtttcaaagctatccccgtgggtaagacgcaagagaatatcgataacaagagactcctggtcgaggacgagaaacgagctgaggactataaaggtgttaaaaagctgctcgaccggtattacctgtccttcattaatgacgttctgcacagcatcaagctcaaaaatctgaacaattacatctccctcttcagaaagaaaacccgaaccgaaaaggagaataaagagttagaaaacctggagatcaaccttcggaaggaaatcgccaaggcattcaagggaaacgagggctacaagagcctgttcaagaaagacatcattgaaactatcttgcccgaattcctggacgataaggacgagattgctctggtcaactccttcaacggcttcaccactgctttcacgggcttctttgacaaccgcgagaacatgtttagcgaggaagcgaagagcacttccatcgcattccgttgtattaacgagaatttgacgcgctacatttccaacatggacattttcgagaaggtagacgcgattttcgacaagcatgaggtccaggaaatcaaggaaaagatcctcaacagtgactatgatgtcgaggatttctttgaaggtgagtttttcaatttcgttctgacccaggaaggtattgacgtttacaacgccatcattggcggtttcgtcactgagtccggagaaaagattaagggtctcaacgagtacatcaacctatacaatcaaaagaccaaacaaaagttgccgaagtttaagcccctctacaagcaggtcctgagtgaccgcgagtcgttgtcgttttacggcgaggggtacaccagcgacgaggaagtgctcgaggttttccggaacacgctcaacaagaacagcgagatcttctccagcatcaagaaattggagaagcttttcaagaactttgacgagtacagttcggctgggatattcgtcaagaacggccccgccatttccaccatctcgaaagatatctttggcgagtggaacgttatcagggacaagtggaacgctgagtacgacgatattcacctgaagaaaaaagccgtggtcacagaaaagtacgaagacgatcgccgtaagagtttcaagaaaatcggttcgttctccctggagcagctccaggagtacgccgacgccgatctatcggttgtcgaaaagctgaaggagatcattattcagaaggtcgacgagatctacaaggtctatggttcctctgaaaagcttttcgatgccgattttgtcctagagaagtctctgaagaaaaatgacgcggtcgttgcgattatgaaggacctcctggactcagtcaagagcttcgagaattacatcaaggctttctttggagaaggtaaagagaccaatagagatgagagcttctatggcgactttgtcctcgcctacgatattctcctgaaggtggaccacatctacgacgccatccgcaactacgtgacccaaaagccatactccaaggataagttcaagctgtatttccagaacccccagtttatgggcgggtgggacaaggacaaggagactgactatcgggctacgattcttcgttatggatctaagtactatttagctatcatggataagaaatatgcaaagtgtttgcagaaaattgacaaagatgacgtgaacggcaactacgagaagatcaactataagcttttgccgggacctaataagatgttgcccaaagtcttcttttccaagaaatggatggcctattacaatcctagcgaggacattcaaaaaatctataaaaacggaacttttaagaaaggagatatgttcaacctaaacgattgccataaacttatcgattttttcaaggatagtattagtcgctacccgaagtggtcgaatgcctacgatttcaacttctcagaaacggaaaagtacaaggacatcgcgggcttctaccgtgaggtggaggaacagggctacaaggttagcttcgagtcagcgtccaagaaagaggtggataagttggtcgaggaaggcaagctgtacatgttccagatctataacaaggacttttctgataagtctcatggcacgcccaatctgcacaccatgtacttcaagcttttgttcgacgaaaacaatcacggccagattcggctctcaggcggagccgagttgttcatgcgccgtgcaagcttgaagaaagaggaactggtcgtgcacccagcgaactcgcccatcgctaacaagaaccctgataaccccaagaaaaccacaaccctgagctatgatgtgtataaagataagcggttctccgaggatcagtacgaactccatattccgatcgctatcaataagtgccccaagaatatcttcaagatcaacaccgaagttcgtgtgctgctcaagcacgatgacaacccctacgtgattggtatcgatcgtggtgaacgcaatcttctgtacattgtcgtggtagatggtaagggaaacatcgtggagcaatattccctcaacgagatcattaacaattttaacggtatcaggataaaaaccgactaccattccctcctggataaaaaggaaaaggagcgtttcgaggcgcgccaaaactggaccagcatcgagaacatcaaggagctgaaggccggttacatttctcaggtggtacacaaaatctgcgagctcgtcgagaaatacgatgctgttattgcccttgaggatctcaatagctactgctcctctgtctaccagaagttcgagaagatgctcatcgacaagctgaattacatggtcgacaagaaatcgaacccttgcgccacaggtggcgccttgaagggttatcagatcactaacaagtttgaatccttcaagagcatgtcaacccaaaacggcttcattttctacatcccggcctggcttaccagcaaaatcgaccctagtacaggtttcgtcaacttgctcaaaactaagtacacgtccatcgcggatagtaagaaattcatatcctctttcgatcggatcatgtacgttcctgaggaagatctgttcgagttcgccctcgattacaagaacttttctcgcaccgacgccgactacatcaagaaatggaagctctattcttacgggaaccgcatccgaatcttccgaaacccaaagaaaaacaatgtcttcgattgggaagaggtgtgcctgacctcggcttacaaggaacttttcaacaaatacgggattaattaccagcaaggagacattcgcgccctgctttgcgagcagtcggataaggccttctattcctcttttatggccctcatgtccctcatgcttcagatgcgcaactccatcaccggtcgtaccgacgttgacttcctcatttctcctgttaagaactccgatggcatattctacgactctcgtaactacgaggcacaggagaatgcgatcctgccgaagaacgcagacgccaacggtgcctacaacatcgcacgtaaggtgctttgggctatcggccagtttaagaaagctgaggatgaaaagttggataaagtcaagatcgctatctccaataaggagtggctagagtacgctcagacttcggtgaaacactaa

SEQ ID NO: 85 - DNA, 3663 bp, synthetic construct
  source 1..3663: mol_type = other DNA; note = LbCas12a RuvC L-del1, codon optimized for Rhodobacter sphaeroides
atgtcaaagctagaaaaatttacaaattgttattctttatcgaaaacactcaggtttaaagctatacccgtaggaaaaacccaggaaaacatagataacaagcgtctgcttgtggaagatgaaaaaagggccgaggattacaaaggagtcaaaaagctccttgaccgatattacctttcctttataaatgatgtcctacactcaattaagttaaagaacttgaacaattatatatcattgtttagaaaaaagacacgtaccgagaaagagaacaaggagttggagaacttggaaataaatttgcgaaaagaaatcgcaaaggcatttaaaggcaacgagggatacaagtcattattcaaaaaggatataattgaaactatactacctgaattcctagatgacaaggacgagatagcactagtgaactcctttaatggatttacaactgcttttactggttttttcgacaatcgtgaaaacatgttttcagaagaggctaaatccacaagcatcgctttcagatgcatcaacgagaacttgacgagatacatctctaacatggatatctttgaaaaggtcgatgcaatttttgataagcacgaagttcaagagataaaagaaaagatattgaattctgattatgatgtggaggattttttcgaaggagagtttttcaacttcgttcttacacaagagggtatagatgtttataatgcaataattggtggatttgtgaccgaaagcggcgagaaaattaagggactgaatgaatacatcaatttgtataaccaaaaaactaaacagaaattacctaaatttaaacctctttataagcaagtcttaagtgatcgcgagtctttatccttttacggggaaggctatacttcggatgaagaggtgctagaggttttcagaaacacccttaataagaactccgaaattttttcgtccataaagaaactggaaaagctgttcaaaaattttgacgaatatagcagtgcgggaatatttgtcaaaaacggaccagcaataagcactattagtaaagatatattcggtgagtggaacgtaattagagataaatggaatgccgaatatgatgacattcatcttaaaaagaaggctgtagttaccgaaaaatatgaggatgaccggaggaaaagttttaagaaaattgggtctttttctttagaacagctgcaagaatatgccgacgccgatctttctgttgtggaaaaacttaaggagataattatacagaaagttgatgagatttataaagtatacggatcctctgaaaaattatttgacgcagatttcgtcttggaaaagagtctaaaaaagaatgatgcagttgtggcgattatgaaggatcttttagatagtgtcaaatctttcgagaactatatcaaggccttctttggtgaggggaaagagacgaaccgcgatgaaagtttctacggtgattttgtcttagcttacgacattctattgaaggtggaccacatctatgatgccatcagaaattatgttactcaaaaaccatatagcaaggataaatttaaattgtattttcaaaatccgcaatttatgggtgggtgggataaagacaaggagacagattatcgagcgacaatcctgagatacgggtctaagtactatctcgctattatggacaaaaagtacgccaagtgcctgcaaaagatcgataaagacgatgttaacggaaattatgaaaaaatcaattataagcttctgccaggaccgaataaaatgctccctaaggtattcttttctaaaaagtggatggcatactataatccatcagaggacattcaaaaaatatataagaatggaaccttcaaaaagggtgacatgtttaaccttaatgattgtcataaactcattgactttttcaaagattctatttccaggtaccctaagtggagcaatgcgtacgattttaatttctcggagacggaaaagtacaaagatatcgcaggcttttatagggaagttgaagagcaaggatataaggtttcattcgaatctgcatctaaaaaggaggttgataagttggtagaagagggcaaattatatatgttccaaatttacaataaagatttttcagacaaaagccatggaacaccgaatctccatactatgtatttcaaactcttgttcgatgaaaataaccacgggcaaattcggttgtcggggggcgctgaactttttatgagaagggccagtttgaaaaaggaggaattggttgtacatcccgctaactcaccgatagcaaataaaaatccagataatccaaaaaagaccactacactatcatacgacgtctataaagacaagcgattctctgaagatcaatatgaattacacattcccatcgcgatcaataagtgtccgaaaaacatttttaagataaatactgaagtaagagtattgctaaaacatgacgataatccctatgttattggtatcgaccggggcgaaagaaacttactctatatagtcgtggttgacgggaaaggaaatattgttgaacagtattccttaaatgaaattataaacaattttaacggtattcgcattaaaacagattatcactcacttttggacaagaaagaaaaggaacgttttgaagcaaggcagaattggacctctatcgagaatattaaagagctaaaggctgggtatattagtcaagttgtgcataaaatttgcgaattagtagaaaaatacgatgcagttattgcactcgaggatctcaacagctattgctcatcggtttaccaaaagttcgagaaaatgcttatcgataaactgaattatatggtagataaaaagagcaatccttgtgccacaggtggcgccctcaaaggttaccaaattactaataaatttgaatcgtttaagtcaatgtcaacacaaaatggattcatattctatatcccagcgtggttaacaagcaagattgaccccagtacgggatttgtaaatttgctgaaaaccaagtatacgtctatcgctgattcgaagaaattcatcagctcctttgataggattatgtatgtacctgaggaagatttatttgagtttgcattagattataaaaacttttcgcgaacggacgcagattatataaaaaagtggaagctttatagttacgggaacagaataagaatttttcggaatcctaaaaagaacaatgtgttcgattgggaggaagtgtgtctgacatctgcttataaagaattatttaataaatacggtataaattatcaacagggcgatatccgggcgttgctctgtgaacagtcagacaaagctttctacagttcatttatggctcttatgtccttgatgctccagatgagaaactcaattactggtaggaccgatgtcgacttccttatttctccagtaaaaaattctgatggcatcttttatgattcacgaaattacgaagcgcaggaaaacgctattttgccaaaaaatgctgatgctaacggagcttataatatagcccgtaaagttttgtgggcaatcgggcagtttaaaaaggcggaggatgagaaactcgataaggtaaaaatagcaatatccaataaagagtggttggagtacgcccaaacgagtgtgaagcattga

SEQ ID NO: 86 - DNA, 3663 bp, synthetic construct
  source 1..3663: mol_type = other DNA; note = LbCas12a RuvC L-del1, codon optimized for Corynebacterium glutamicum
atgtccaagctggagaagttcaccaactgttactccctgtccaagactctccgcttcaaggctatcccagtcggtaagacccaggaaaacatcgataacaagcgcctgttggtagaggacgagaagcgtgcagaggactacaagggcgttaagaaacttctggaccgctactatttgagcttcatcaacgacgtgttgcactccattaagctgaagaacctgaacaattacatctccctcttccgtaagaaaacccgtaccgaaaaggagaacaaggaacttgagaacctggaaatcaaccttcgcaaggagatcgctaaggctttcaaaggcaacgagggctacaagagcctgttcaaaaaggacatcattgagaccatcctgccggagttcctggacgataaggacgagatcgctctcgtaaactccttcaacggcttcaccactgcttttaccggcttctttgacaatcgcgaaaacatgttctccgaggaagctaagagcactagcatcgcattccgctgcatcaacgaaaacctcacccgctatatctctaacatggacattttcgagaaggtcgacgcaatcttcgacaagcacgaggtccaggaaatcaaggagaagattctgaacagcgactacgacgttgaggacttctttgaaggcgagtttttcaacttcgtcttgacccaggagggcatcgacgtttacaacgctatcattggaggcttcgttaccgagtccggcgagaagatcaagggtcttaacgagtacatcaacctttacaaccagaagaccaagcagaagctgcctaagttcaagccactgtacaagcaggtgctcagcgaccgcgagtctctctccttctacggtgagggctacacctccgacgaggaagttctcgaagttttccgcaacaccctcaacaagaactccgaaatcttttcctctatcaagaaactggaaaagctgttcaagaatttcgacgagtactcctctgccggcatcttcgttaaaaacggtccagcaatctccaccatctccaaggatatcttcggcgagtggaacgttatccgtgacaagtggaacgctgaatacgatgacattcacctaaagaaaaaagccgttgtcaccgagaagtacgaggacgatcgccgtaagtccttcaagaaaatcggctccttctccctggagcagctgcaggaatacgctgatgcagacctctccgtcgttgagaagctgaaggagattatcattcagaaggtcgatgagatctacaaggtttacggctcctctgagaagctctttgacgcagatttcgtccttgagaagtccctgaagaaaaacgatgcggttgtcgctatcatgaaggacctcctggattccgttaagtccttcgagaactacatcaaggctttctttggcgagggtaaggagaccaaccgcgacgagtccttctacggtgacttcgttctcgcatacgacatcctgcttaaggttgaccacatttacgatgcaatccgcaactacgtgactcagaagccttactccaaggacaagttcaaactgtacttccagaacccacagttcatgggcggttgggataaggataaggagaccgactaccgcgcgactatcctccgttacggatctaagtactatctggcaatcatggacaagaaatacgcaaagtgccttcagaagatcgacaaggacgatgttaatggaaactacgagaagatcaattacaagcttctccctggccctaacaagatgctgccaaaggtttttttctctaagaaatggatggcatactataacccttctgaagatatccagaagatctacaagaatggcaccttcaagaaaggtgacatgttcaacctgaacgactgccacaaactgatcgacttctttaaggattccatctcccgctacccaaagtggtccaacgcttacgactttaacttctccgagaccgaaaagtacaaggacattgccggtttctaccgtgaggttgaagagcagggctacaaggtctccttcgaatccgcatcgaagaaagaggttgacaagctcgtcgaggaaggtaagctgtacatgttccaaatttacaacaaggatttctccgataagtcccacggcaccccaaaccttcacaccatgtacttcaaacttctatttgacgaaaacaatcacggccagatccgcctgtccgggggtgctgagttgttcatgcgtcgcgcttccctcaagaaagaggaactggtcgttcatccagctaactccccaatcgcaaataagaacccagataaccctaagaaaaccactaccctgtcctacgacgtctacaaggacaagcgtttctccgaggaccagtacgaactgcacatcccaatcgcaatcaacaagtgcccaaagaatattttcaaaatcaacaccgaggtccgtgtactgctcaagcacgacgataacccttacgttatcggaatcgaccgtggtgaacgcaacttgctttacatcgttgtagttgatggtaagggcaacatcgtcgagcagtactctctgaacgagatcattaacaatttcaacggcatccgcattaagactgattaccactctctgctcgacaagaaagagaaggagcgcttcgaggcacgtcagaactggaccagtatcgaaaacatcaaggagcttaaggctggttacatctcccaggtggttcacaagatctgtgaattggttgagaagtacgacgccgttattgctctggaagatctcaattcatactgctcctctgtctaccagaagttcgaaaagatgctcattgataagcttaactacatggtcgacaagaaatccaacccctgcgcaaccggcggtgctctcaagggctaccagatcactaacaagttcgaatccttcaagtccatgtccacccagaacggcttcatcttctacatcccggcttggctcacctctaagatcgatccttcaaccggtttcgtgaacctcctgaagaccaagtacacttcgatcgctgattctaagaaattcatctctagcttcgaccgcattatgtacgtcccagaagaggatctgttcgagttcgctcttgactacaagaacttttctcgtaccgacgcggactacatcaagaaatggaagctttactcctatggtaaccgtatccgtatcttccgtaacccaaagaaaaacaatgttttcgactgggaggaagtgtgcctgacctctgcttacaaggagctgttcaacaaatacggtattaactaccagcaaggcgatatccgagctctcttgtgcgaacagtccgataaagcattctactcctctttcatggcccttatgtccctgatgcttcaaatgcgcaactctatcaccggccgcaccgacgtcgattttctcatttctcctgtcaagaactccgacggcatcttctatgattctcgcaactacgaggcacaggaaaacgcgatccttcctaagaatgctgacgctaacggtgcttacaacatcgcacgtaaggtcctgtgggccatcggccagttcaagaaagcagaagatgagaagctggacaaggtgaagatcgctatctccaacaaggagtggctagagtacgcacagacctccgttaagcactaa

SEQ ID NO: 87 - DNA, 3663 bp, synthetic construct
  source 1..3663: mol_type = other DNA; note = RuvC L-del1, codon optimized for Pseudozyma tsukubaensis
atgagtaagctcgagaagtttacaaattgctatagtttgagtaaaacgttgaggttcaaggccatccccgtcggaaagacacaagagaacattgacaacaagcgtctcttggttgaagacgagaagcgcgccgaagactacaagggggttaagaaactcttggaccgatattaccttagttttatcaacgacgtcctccattctatcaaactcaagaacttgaataactatatctcgctatttcgtaagaaaacacgtacggagaaagaaaataaggagcttgagaacctcgagatcaacctacggaaggagattgccaaggccttcaagggcaacgaaggctacaagtctctcttcaagaaagacatcattgaaacgatcctcccagagtttctcgatgacaaggacgaaatcgctcttgtcaactctttcaatggctttacgaccgctttcaccggcttctttgataaccgagaaaacatgttttcggaagaggccaaatcaacttcgattgcttttcgctgcatcaacgagaatctcaccaggtacatcagcaacatggatattttcgagaaggtcgatgcgatctttgacaagcacgaagtccaagaaatcaaggaaaagatcctgaactcggactatgatgttgaggacttctttgaaggtgagttctttaacttcgtcctgactcaggagggaatcgatgtatacaacgctatcattggtggcttcgtcacggagtcaggcgagaagatcaaggggctgaacgagtacatcaatctctataaccagaagactaaacaaaagctcccaaagtttaagcccctctacaagcaggtcttgtctgatcgtgaaagcctgtctttctatggggaaggttacacctcagacgaagaggtgcttgaggtgttccgtaacaccttgaacaaaaactccgaaatcttctccagcattaagaaactcgagaaactctttaaaaacttcgatgaatatagttccgctggtattttcgtcaagaacggaccagccatctcgactatctccaaagacatcttcggtgagtggaatgtaatcagagacaaatggaatgcagaatacgatgacattcaccttaaaaagaaggctgttgtcacagaaaaatacgaagacgatcgacgtaagtctttcaagaaaattggatctttcagcttggagcaattgcaggagtacgcagacgcagacctaagcgtcgtggagaagctgaaggagatcattattcagaaggttgacgagatttacaaggtttacggctcctcggaaaaactctttgacgcggatttcgtcttggagaagtctctcaagaaaaacgacgctgtggtcgcaatcatgaaggatctcctggatagcgtaaagtcgttcgagaattacattaaagcttttttcggtgaaggtaaggaaaccaacagggatgaaagcttttatggcgatttcgttctggcgtacgatatcctcttgaaggtggaccatatctacgacgcaatccgcaactacgtcactcagaagccctactccaaggataagttcaaactttacttccaaaatcctcagttcatgggcggttgggacaaggataaggaaaccgattaccgagctactatcctccgctatggctccaaatactatttggcaatcatggataaaaagtacgcaaagtgcctccagaagatcgacaaagatgacgttaatgggaactatgagaagattaactataaactactgcctggcccgaacaaaatgttgccgaaagtgtttttcagcaagaaatggatggcatactataacccgagcgaggacatccagaaaatctataaaaacggaacattcaagaaaggagatatgtttaaccttaacgactgccataagttgattgattttttcaaggactcaatctctcgctaccctaagtggtcgaacgcttacgatttcaacttttcagagaccgagaagtataaagacatcgccggattctacagagaagtggaggaacagggttacaaggtcagcttcgaatcagcgtcgaaaaaggaggtcgacaagctcgtggaggaaggcaagttgtacatgttccaaatctacaataaggatttctcagacaaatctcacggcactccaaacctgcatacaatgtactttaaactcttgttcgacgaaaacaatcacggccaaatacgcctatccggtggcgcagagctcttcatgcgacgcgcctcccttaaaaaggaagagcttgttgtgcatcctgcgaattcacctattgctaacaagaacccggataatcccaagaaaaccacaaccttgtcctacgacgtctacaaggacaaacgcttcagtgaggatcagtacgaacttcacatccctatcgccatcaacaagtgtcctaagaatattttcaagattaataccgaagtccgagtgctccttaaacacgacgataatccctacgtaatcggcatcgacaggggcgaacggaacctgttgtacattgttgtcgtagatggcaagggaaacatcgtcgagcagtactcgctcaatgagatcattaacaatttcaacggaatccgcattaagaccgattaccactcgctgcttgataaaaaggaaaaggagcgtttcgaagccaggcaaaattggacatcaatcgaaaacatcaaggaactcaaggctggttatatttctcaagtggtccacaagatttgcgaattggtggagaagtatgacgcagtcatagctttggaggatttaaattcctattgtagctcggtctaccaaaagttcgaaaaaatgcttattgacaagcttaactatatggtagataaaaagtctaatccatgcgcgacgggtggcgcccttaagggttaccagatcacgaacaagttcgagtccttcaagtcgatgtccacgcagaacggcttcattttctacataccagcatggctgacgtcgaaaattgatccttcgacaggtttcgtcaacctccttaagacgaaatatactagcatcgccgattcgaaaaagtttatcagctcgttcgatcgcatcatgtacgtcccggaggaagacctcttcgagttcgcactcgattacaagaacttttccagaaccgacgctgactacatcaaaaagtggaagctttacagctacggtaacaggattcgcatcttccgtaaccccaagaaaaacaatgtcttcgactgggaggaagtgtgcctcaccagcgcatataaggagctcttcaataagtacggaatcaactaccaacagggcgacatcagagcgctgctctgtgaacagtcggacaaagccttttactcctcgttcatggctttgatgagtcttatgctccagatgagaaattcaataaccggccgtaccgacgttgactttttgatctcgcctgtaaagaattcggatggaatcttctacgacagccgaaactacgaggcgcaggagaacgcgatcctccccaagaacgcagacgccaacggggcgtacaacatcgcccgcaaggtcttatgggctatcggacagttcaagaaagctgaagatgaaaagctggataaggtcaaaatcgccatttcgaacaaggagtggctggaatacgcccaaacttcggtcaaacattga

SEQ ID NO: 88 - DNA, 3663 bp, synthetic construct
  source 1..3663: mol_type = other DNA; note = RuvC L-del1, codon optimized for Glycine max
atgtcaaaattggaaaaattcacaaattgttattctttgagcaaaacattgagattcaaagcaattcctgttggaaaaactcaagaaaacattgacaacaaaagattgttggtggaagatgaaaaaagagcagaagattacaaaggagtgaagaaattgttggacagatattatttgagcttcatcaatgatgttcttcattcaatcaaattgaagaatttgaacaattacatttctctcttcagaaagaaaacaagaacagaaaaagaaaacaaagaattggaaaatttggaaatcaatttgagaaaagaaattgcaaaagcattcaaaggaaatgaaggttacaaatcattgttcaagaaagacatcattgaaacaattcttccagaatttcttgatgacaaagatgaaattgctttggtgaacagcttcaatggattcacaactgctttcactggattctttgacaacagagaaaacatgttttcagaagaagcaaaatcaacaagcattgctttcagatgcatcaatgaaaatttgactcgttacatttcaaacatggatatttttgaaaaagttgatgcaatttttgacaaacatgaagttcaagaaatcaaagaaaagattctcaattcagattatgatgttgaagatttctttgaaggagaatttttcaattttgttttgacacaagaaggaattgatgtttacaatgcaatcattggaggatttgtgacagaaagtggagaaaagatcaaaggattgaatgaatacatcaatttgtacaaccaaaaaacaaaacaaaaattgccaaaattcaaacctctttacaaacaagttctttcagacagagaaagtttgagcttctatggagaaggatatacttctgatgaagaagttttggaagttttcagaaacactttgaacaaaaattcagaaattttttcttcaatcaagaaattggagaagctcttcaagaattttgatgaatattcttctgctggaatttttgtgaagaatggaccagcaatttcaacaatttcaaaagatatttttggagaatggaatgtgatcagagacaaatggaatgctgaatatgatgatattcatttgaagaagaaagctgttgtgacagaaaaatatgaagatgacagaagaaaaagtttcaagaagattggaagtttttctttggaacaacttcaagaatatgctgatgctgatctttcagttgttgaaaaattgaaagaaatcatcattcaaaaagttgatgaaatttacaaagtttatggaagttcagaaaaattgtttgatgctgattttgttttggagaaaagtttgaagaagaatgatgctgttgttgcaatcatgaaagatttgttggattcagtgaagagttttgaaaattacatcaaagcattctttggagaaggaaaagaaacaaacagagatgaaagtttttatggagattttgttcttgcttatgatattttgttgaaggttgatcacatttatgatgcaatcagaaattatgtcactcaaaaaccatattcaaaagacaaattcaaactttattttcaaaatcctcaattcatgggaggatgggacaaagacaaagaaacagattacagagcaacaattttgagatatggatcaaaatattatttggcaatcatggacaagaaatatgcaaaatgcttgcagaagattgacaaagatgatgtgaatggaaattatgaaaagatcaattacaaattgcttcctggaccaaacaagatgcttccaaaagttttcttctcaaagaaatggatggcatattacaatccttcagaagatattcaaaagatttacaagaatggaactttcaagaaaggagatatgttcaatttgaatgattgccacaaattgattgatttcttcaaagattcaatttcaagatacccaaaatggagcaatgcttatgatttcaatttttcagaaacagaaaaatacaaagatattgctggattttacagagaagttgaagaacaaggatacaaagtgagttttgaaagtgcttcaaagaaagaagttgacaaattggtggaagaaggaaaactttatatgtttcaaatttacaacaaagatttttcagacaaatctcatggaactccaaatcttcacacaatgtatttcaaattgttgtttgatgaaaacaatcatggacaaatcagattgagtggaggagcagaattgttcatgagaagagcttcattgaagaaagaagaattggtggttcatccagcaaattctccaattgcaaacaaaaatccagataatccaaagaaaacaacaactctttcttatgatgtttacaaagacaaaagattttcagaagatcaatatgaacttcacattccaattgcaatcaacaaatgtccaaagaacattttcaaaatcaacacagaagtgagagttttgttgaagcatgatgacaatccatatgtgattggaattgacagaggagaaagaaatcttctttacattgttgttgttgatggaaaaggaaacattgttgaacaatattcattgaatgaaatcatcaacaatttcaatggaatcagaatcaaaacagattatcattctttgttggacaagaaagaaaaagaaagatttgaagcaagacaaaattggacttcaattgaaaacatcaaagaattgaaagctggatatatttctcaagttgttcacaaaatttgtgaattggtggaaaaatatgatgctgtgattgctttggaagatttgaactcttattgttcttctgtttatcaaaaatttgagaagatgttgattgacaaattgaattacatggtggacaagaaatcaaatccttgtgcaactggtggagctttgaaaggttatcaaatcacaaacaaatttgaaagtttcaaatcaatgtcaactcaaaatggattcatcttctacattcctgcttggttgacaagcaaaattgatccttcaactggatttgtgaatttgttgaaaacaaaatacacttcaattgctgattcaaagaaattcatttcttcttttgacagaataatgtatgttccagaagaagatttgtttgaatttgctttggattacaagaatttttcaagaactgatgctgattacatcaagaaatggaaactttattcttatggaaacagaatcagaattttcagaaatccaaagaagaacaatgtttttgattgggaagaagtttgtttgacttcagcttacaaagaattgttcaacaaatatggaatcaattatcaacaaggagacatcagagctcttctttgtgaacaaagtgacaaagcattttattcttctttcatggctttgatgagtttgatgcttcaaatgagaaattcaatcactggaagaactgatgttgatttcttgatttctccagtgaaaaattctgatggaattttctatgattcaagaaattatgaagctcaagaaaatgcaattcttccaaagaatgctgatgcaaatggagcttacaacattgcaagaaaagttctttgggcaattggacaattcaagaaagcagaagatgaaaaattggacaaagtgaagattgcaatttcaaacaaagaatggttggaatatgctcaaacttctgtgaagcattaa

SEQ ID NO: 89 - DNA, 3663 bp, synthetic construct
  source 1..3663: mol_type = other DNA; note = LbCas12a RuvC L-del1, codon optimized for Zea mays
atgagcaagctggagaagttcaccaactgctacagcctctccaagacgctgcgcttcaaggccatccccgtcggcaagacgcaggagaacatcgacaacaagcggctgctggtggaggatgaaaagagggcggaggactacaagggcgtcaagaagctgctggaccgctactacctcagcttcatcaacgacgtgctgcactcaatcaagctgaagaacctcaacaactacatcagcctcttccgcaagaagacgcggacggagaaggagaacaaggagctggagaacctggagatcaacctccgcaaggagatcgccaaggccttcaagggcaatgagggctacaagagcctcttcaagaaggacatcatcgagacgatcctaccggagttcctggacgacaaggatgagatcgcgctggtgaacagcttcaacggcttcaccaccgccttcaccggcttcttcgacaaccgggagaacatgttctcagaggaggccaagagcacctccatcgccttccgctgcatcaacgagaacctgacgcgctacatcagcaacatggacatcttcgagaaggtggacgccatcttcgacaagcatgaggtccaggagatcaaggagaagatcctcaactcagactacgacgtggaggacttcttcgagggcgagttcttcaacttcgtgctgacgcaggagggcattgatgtctacaacgccatcatcggcggcttcgtcaccgagtcaggggagaagatcaagggcctcaacgagtacatcaacctctacaaccagaagaccaagcagaagctgcccaagttcaagccgctctacaagcaggtgctctccgaccgggagagcctgagcttctacggcgagggctacacctcagatgaggaggtgctggaggtgttccgcaacaccctcaacaagaacagcgagatcttcagcagcatcaagaagctggagaagctcttcaagaacttcgacgagtacagctccgccggcatcttcgtgaagaacgggccggccatctccaccatcagcaaggacatcttcggcgagtggaacgtcatccgcgacaagtggaacgccgagtacgatgacatccacctaaagaagaaggcggtggtgacggagaaatatgaagatgaccgccgcaagagcttcaagaagatcggcagcttctcgctggagcagctccaggagtacgccgacgccgacctctccgtggtggagaagctgaaggagatcatcatccagaaggtggacgagatctacaaggtgtacggcagctcagagaagctcttcgacgctgacttcgtgctggagaagagcctcaagaagaacgacgccgtggtggccatcatgaaggacctgctggactcagtgaagagcttcgagaactacatcaaggccttcttcggcgagggcaaggagacgaaccgcgacgagagcttctacggcgacttcgtgctggcctacgacatcctgctgaaggtggaccacatctatgacgccatccgcaactacgtcacccagaagccatactcaaaggacaagttcaagctctacttccagaacccgcagttcatgggcggctgggacaaggacaaggagacggactaccgcgccaccatcctgaggtatggctcaaagtactacctcgccatcatggacaagaagtacgccaagtgcctgcagaagatcgacaaggatgatgtgaacggcaactacgagaagatcaactacaagctgctgccggggccgaacaagatgctgcccaaggtgttcttcagcaagaagtggatggcctactacaacccctcagaggacatccagaagatctacaagaacggcaccttcaagaagggcgacatgttcaacctcaatgactgccacaagctgatcgacttcttcaaggacagcatcagccgctaccccaagtggagcaacgcctatgacttcaacttctcagagacggagaagtacaaggacatcgccggcttctaccgcgaggtggaggagcagggctacaaggtgagcttcgagagcgccagcaagaaggaggtggacaagctggtggaggagggcaagctctacatgttccagatctacaacaaggacttctcagacaagagccacggcacgcccaacctccacaccatgtacttcaagctgctgtttgacgagaacaatcacggccagatccgcctctcaggcggcgccgagctcttcatgaggcgcgcgtcgctgaagaaggaggagctggtggtgcaccccgccaacagccccatcgccaacaagaacccggacaaccccaagaagacgacgacgctgagctacgacgtctacaaggacaagcgcttctcagaggaccagtatgagctgcacatccccatcgccatcaacaagtgccccaagaacatcttcaagatcaacaccgaggtgcgcgtgctgctgaagcatgacgacaacccctatgtcatcggcatcgaccgcggcgagcgcaacctgctatacatcgtggtggtggacggcaagggcaacatcgtggagcagtacagcctgaacgagatcatcaacaacttcaacggcatccgcatcaagacggactaccactcactgctggacaagaaggagaaggagcgctttgaggcgcggcagaactggacctccatcgagaacatcaaggagctgaaggccggctacatcagccaggtggtgcacaagatctgcgagctggtggagaagtatgacgccgtcatcgcgctggaggacctcaacagctactgctcaagcgtctaccagaagttcgagaagatgctcatcgacaagctcaactacatggtggacaagaagagcaacccctgcgccaccggcggcgcgctgaagggctaccagatcaccaacaagtttgagagcttcaagagcatgtcaacccagaacggcttcatcttctacatccccgcctggctcaccagcaagattgatccctccaccggcttcgtcaacctgctgaagaccaagtacacctccatcgccgacagcaagaagttcatcagcagcttcgaccgcatcatgtacgtgccggaggaggacctcttcgagttcgcgctggactacaagaacttctcaaggaccgacgctgactacatcaagaagtggaagctctacagctacggcaaccggataaggatcttccgcaaccccaagaagaacaacgtcttcgactgggaggaggtctgcctcacctccgcctacaaggagctcttcaacaagtacggcatcaactaccagcagggcgacatccgcgcgctgctatgtgagcagagcgacaaggccttctacagcagcttcatggcgctgatgagcctgatgctgcagatgcgcaacagcatcaccggccgcaccgacgtggacttcctcatctcgccggtgaagaacagcgacggcatcttctacgactcaaggaactacgaggcgcaggagaacgccatcctccccaagaacgctgacgccaacggcgcctacaacatcgcgcgcaaggtgctctgggccatcggccagttcaagaaggcggaggacgagaagctggacaaggtgaagatcgccatcagcaacaaggagtggctggagtacgcgcagacctccgtcaagcattaa

SEQ ID NO: 90 - DNA, 3663 bp, synthetic construct
  source 1..3663: mol_type = other DNA; note = LbCas12a RuvC L-del1, codon optimized for Brassica napus
atgtcaaagctggagaagttcacaaactgctactctctctcaaagactttgagattcaaagctattcctgttggaaagactcaagagaacattgacaacaagagacttcttgttgaagatgagaagagagctgaagattacaaaggagtgaagaagctcttggacagatattatctctctttcatcaatgatgttcttcattcaatcaagctgaagaatctcaacaactacatctctctcttcagaaagaagacaagaacagagaaggagaacaaagagctggagaacttggagatcaacttgagaaaggagattgcaaaggctttcaaaggaaatgaaggttacaaatcactcttcaagaaagacatcattgaaaccattcttccagagtttcttgatgacaaagatgagattgctttggtgaacagcttcaatggtttcacaacagctttcactggtttctttgacaacagagagaacatgttctcagaagaagcaaaatcaacaagcattgctttcagatgcatcaatgagaacttgactcgttacatctcaaacatggacatctttgagaaggttgatgcaatctttgacaaacatgaagttcaagagatcaaagagaagattctcaactcagattatgatgttgaagatttctttgaaggagagttcttcaactttgttctcactcaagaaggaattgatgtttacaatgcaatcattggaggatttgtgacagagagtggagagaagatcaaaggtttgaatgagtacatcaatctctacaatcagaagacaaagcagaagctgccaaagttcaaacctctctacaaacaagttctctcagacagagagagcttgagcttctatggagaaggatacacttcagatgaagaagttcttgaagtcttcagaaacactctcaacaagaactcagagatcttctcttcaatcaagaagctggagaagctcttcaagaactttgatgaatattcttcagctggaatctttgtgaagaatggaccagcaatctcaacaatctccaaagacatctttggagaatggaatgtgatcagagacaaatggaatgctgagtatgatgatattcatctcaagaagaaagctgtggtgacagagaaatatgaagatgacagaagaaagagcttcaagaagattggaagtttctcattggagcagcttcaagaatatgctgatgctgatctctctgttgtggagaagctgaaggagatcatcattcagaaggttgatgagatctacaaggtttatggaagctcagagaagctctttgatgctgattttgttttggagaagagcttgaagaagaatgatgctgttgttgcaatcatgaaagatctcttggattcagtgaagagctttgagaactacatcaaggctttctttggagaaggaaaagaaacaaacagagatgagagcttctatggagattttgttcttgcttatgatattcttctcaaggttgatcacatctatgatgcaatcagaaactatgtgactcagaagccttactcaaaagacaagttcaagctctactttcaaaatcctcagttcatgggaggatgggacaaagacaaagaaacagattacagagcaacaatcttgagatatggatcaaagtactatcttgcaatcatggacaagaagtatgcaaaatgtcttcagaagattgacaaagatgatgtgaatggaaactatgagaagatcaactacaagctgcttcctggaccaaacaagatgcttccaaaggtcttcttctcaaagaagtggatggcttactacaatccttcagaagacattcagaagatctacaagaatggaactttcaagaaaggagacatgttcaatctcaatgattgtcacaagctgattgatttcttcaaagattcaatctcaagatacccaaaatggagcaatgcttatgatttcaacttctcagaaacagagaagtacaaagacattgctggtttctacagagaagttgaagaacaaggatacaaggtgagctttgaatcagcttcaaagaaagaagttgacaagctggtggaagaaggaaagctctacatgtttcagatctacaacaaagatttctcagacaagagtcatggaactccaaatcttcacacaatgtacttcaagctcttgtttgatgagaacaatcatggacagatcagactctcaggaggagctgagctcttcatgagaagagcttctctcaagaaagaagagcttgttgttcatccagcaaactctccaattgcaaacaagaatccagacaatccaaagaagacaacaactctctcttatgatgtttacaaagacaagagattctcagaagatcaatatgagcttcacattcccattgcaatcaacaaatgtccaaagaacatcttcaagatcaacacagaagtgagagttcttctaaaacatgatgacaatccttatgtgattgggattgacagaggagaaagaaatcttctctacattgttgttgttgatggaaaaggaaacattgttgaacaatattctctcaatgagatcatcaacaacttcaatgggataagaatcaagacagattatcattcacttcttgacaagaaggagaaagaaagatttgaagcaagacaaaactggacaagcattgagaacatcaaagagctcaaagctggttacatctctcaagttgttcacaagatttgtgagctggtggagaagtatgatgctgtgattgctttggaagatctcaacagctattgttcttcagtttatcagaagtttgagaagatgttgattgacaagctcaactacatggtggacaagaagagcaatccttgtgcaactggaggagctctcaaaggttatcagatcacaaacaagtttgagagcttcaagagcatgtcaactcaaaatggtttcatcttctacattccagcttggttgacttcaaagattgatccttcaactggttttgtgaatctcttgaagacaaagtacacaagcattgctgattcaaagaagttcatctcttcttttgacagaatcatgtatgttccagaagaagatctctttgagtttgctcttgattacaagaacttctcaagaacagatgctgattacatcaagaagtggaagctctactcttatggaaacaggataagaatcttcagaaatccaaagaagaacaatgtctttgattgggaagaagtttgcttgacttcagcttacaaagagctcttcaacaagtatggaatcaactatcaacaaggagacatcagagctcttctttgtgaacaaagtgacaaggctttctactcttctttcatggctttgatgagcttgatgcttcagatgagaaactccatcactggaagaacagatgttgatttcttgatctctccggtgaagaactctgatggaatcttctatgattcaagaaactatgaagctcaagagaatgctattcttccaaagaatgctgatgcaaatggagcttacaacattgcaagaaaggttctttgggccattggacagttcaagaaagctgaagatgagaagctggacaaggtgaagattgcaatctcaaacaaagaatggttggagtatgctcaaacttctgtgaagcattaa

SEQ ID NO: 91 - DNA, 3663 bp, synthetic construct
  source 1..3663: mol_type = other DNA; note = LbCas12a RuvC L-del1, codon optimized for Gossypium spp.
atgtcaaaattggaaaaatttacaaattgttattctctttcaaaaactttgagattcaaagcaattcctgttggaaaaactcaagaaaacattgacaacaaaagattgcttgttgaagatgaaaaaagagctgaagattacaaaggagtgaagaaattgttggacagatattatctttctttcatcaatgatgttcttcattcaatcaaattgaagaatttgaacaattacatttctttgttcagaaagaaaacaagaacagaaaaagaaaacaaagaattggaaaatttggaaatcaatttgagaaaagaaattgcaaaagcattcaaaggaaatgaaggttacaaatcattgttcaagaaagatattattgaaacaattcttccagaatttcttgatgacaaagatgaaattgctttggtgaactctttcaatggatttacaactgctttcactggtttctttgacaacagagaaaacatgttttcagaagaagcaaaatcaacaagcattgctttcagatgcatcaatgaaaatttgactcgttacatttcaaacatggatatttttgaaaaagttgatgcaatttttgacaaacatgaagttcaagaaatcaaagaaaagattctcaattcagattatgatgttgaagatttctttgaaggagaatttttcaattttgttttgactcaagaaggaattgatgtttacaatgcaatcattggaggatttgttacagaaagtggagaaaaaatcaaaggattgaatgaatatatcaatctttacaatcaaaaaacaaaacaaaaacttccaaaattcaaacctctttacaaacaagttctttcagacagagaaagtttgagcttttatggagaaggatatacttctgatgaagaagttttggaagttttcagaaacactttgaacaaaaattcagaaattttttcttcaatcaagaaattggaaaaacttttcaaaaattttgatgaatattcttctgctggaatttttgtgaagaatggaccagcaatttcaacaatttcaaaagatatttttggagaatggaatgttatcagagacaaatggaatgctgaatatgatgatattcatttgaagaagaaagctgttgttacagaaaaatatgaagatgacagaagaaaaagtttcaagaagattggaagtttttctttggaacaacttcaagaatatgctgatgctgatctttcagttgttgaaaaattgaaagaaatcatcattcaaaaagttgatgaaatttacaaagtttatggaagttcagaaaaattgtttgatgctgattttgttttggaaaaaagtttgaagaaaaatgatgctgttgttgcaatcatgaaagatttgttggattcagtgaaaagttttgaaaattacatcaaagcattttttggagaaggaaaagaaacaaacagagatgaaagtttttatggagattttgttcttgcttatgatattttgttgaaagttgatcatatttatgatgcaatcagaaattatgttactcaaaaaccatattcaaaagacaaattcaaactttattttcaaaatcctcaattcatgggaggatgggacaaagacaaagaaacagattacagagcaacaattttgagatatggttcaaaatattatttggcaatcatggacaagaaatatgcaaaatgtttgcaaaagattgacaaagatgatgtgaatggaaattatgaaaaaatcaattacaaattgcttcctggaccaaacaagatgcttccaaaagttttcttttcaaagaaatggatggcttattacaatccttcagaagatattcaaaagatttacaaaaatggaactttcaagaaaggagatatgttcaatttgaatgattgccacaaattgattgatttcttcaaagattcaatttcaaggtatccaaaatggagcaatgcttatgatttcaatttttcagaaacagaaaaatacaaagatattgctggattttacagagaagttgaagaacaaggatacaaggtgagctttgaaagtgcttcaaagaaagaagttgacaaattggtggaagaaggaaaactttacatgtttcaaatttacaacaaagatttttcagacaaatctcatggaactccaaatcttcatacaatgtatttcaaattgctttttgatgaaaacaatcatggacaaatcagattatctggtggagctgaacttttcatgagaagagcttctttgaagaaagaagaattggttgttcatccagcaaattctccaattgcaaacaaaaatccagataatccaaagaaaacaacaactctttcttatgatgtttacaaagacaaaagattttcagaagatcaatatgaacttcatattccaattgcaatcaacaaatgtccaaaaaacattttcaaaatcaacactgaagtgagagttttgttgaagcatgatgacaatccttatgttattggaattgacagaggagaaagaaatcttctttacattgttgttgttgatggaaaaggaaacattgttgaacaatattcattgaatgaaatcattaacaatttcaatggaatcagaatcaaaacagattatcattctttgttggacaagaaagaaaaagaaagatttgaagcaagacaaaattggacttcaattgaaaacatcaaagaattgaaagctggatatatttctcaagttgttcacaagatttgtgaattggtggaaaaatatgatgctgttattgctttggaagatttgaactcttattgttcttctgtttatcaaaaatttgaaaagatgttgattgacaaattgaattacatggtggacaagaaatcaaatccttgtgcaactggtggagctttgaaaggttatcaaatcacaaacaaatttgaaagtttcaaatcaatgtcaactcaaaatggtttcattttttacattcctgcttggttgacttcaaagattgatccttcaactggatttgtgaatttgttgaaaacaaaatatacttcaattgctgattcaaagaaattcatttcttcttttgacagaatcatgtatgttccagaagaagatttgtttgaatttgctttggattacaaaaatttttcaagaactgatgctgattacatcaagaaatggaaactttattcttatggaaacagaataagaattttcagaaatccaaagaaaaacaatgtttttgattgggaagaagtttgtttgacttcagcttacaaagaacttttcaacaaatatggaatcaattatcaacaaggagacatcagagctttgctttgtgaacaaagtgacaaagcattttattcttctttcatggctttgatgagcttgatgcttcaaatgagaaattcaatcactggaagaactgatgttgattttttgatttctccagtgaaaaattctgatggaattttttatgattcaagaaattatgaagctcaagaaaatgcaattcttccaaaaaatgctgatgcaaatggagcttacaacattgcaagaaaagttctttgggcaattggacaattcaagaaagctgaagatgaaaaattggacaaggtgaagattgcaatttcaaacaaagaatggttggaatatgctcaaacttcagtgaagcattaa

SEQ ID NO: 92 - DNA, 3663 bp, synthetic construct
  source 1..3663: mol_type = other DNA; note = LbCas12a RuvC L-del1, codon optimized for Oryza sativa
atgagcaagctggagaagttcaccaactgctacagcttgagcaagacattgaggttcaaggccattcctgttggaaaaacacaagagaacattgacaacaagaggctgctggtggaagatgagaagagagcagaagattacaaaggagtgaagaagctgctggacagatattatctctcattcatcaatgatgtgctgcacagcatcaagctgaagaacctcaacaactacatcagcttgttcaggaagaagacaagaacagagaaggagaacaaggagctggagaacttggagatcaacttgaggaaggagattgccaaggcattcaagggaaatgaaggctacaaatcattgttcaagaaggacatcattgaaacaattcttccagagttcttggatgacaaggatgagattgctctggtgaacagcttcaatggcttcaccacagcattcaccggcttcttcgacaacagagagaacatgttctcagaagaagcaaagagcacaagcattgcattcagatgcatcaatgagaacttgacacgctacatcagcaacatggacatctttgagaaggtggatgccatcttcgacaagcatgaagttcaagagatcaaggagaagatcctcaattcagattatgatgtggaggacttctttgaaggagagttcttcaactttgtgctgacacaagaaggaattgatgtgtacaatgccatcattggaggatttgtcacagaaagtggagagaagatcaagggcctcaatgagtacatcaacctctacaaccagaagaccaagcagaagctgccaaagttcaagccattgtacaagcaggtgctctcagacagagagagcttgagcttctatggagaaggatacacatcagatgaagaagtgctggaggtgttcaggaacaccctcaacaagaactcagagatcttcagcagcatcaagaagctggagaagctcttcaagaactttgatgaatattcttctgctggcatctttgtgaagaatgggccggccatcagcaccatcagcaaggacatctttggagaatggaatgtcatcagagacaaatggaatgcagaatatgatgacatccatctaaagaagaaggctgtggtgacagagaaatatgaagatgacagaaggaagagcttcaagaagatcggcagcttcagcttggagcagctgcaagaatatgctgatgctgatctctctgtggtggagaagctcaaggagatcatcatccagaaggtggatgagatctacaaggtgtatggaagctcagagaagctctttgatgctgattttgtgctggagaagagcttgaagaagaatgatgctgtggtggccatcatgaaggatctgctggattcagtgaagagctttgagaactacatcaaggcattctttggagaaggaaaagaaacaaacagagatgagagcttctatggagattttgtgctggcatatgacatcttgctgaaggtggatcacatctatgatgccatcaggaattatgtcacacagaagccatattcaaaggacaagttcaagctctacttccagaatccacaattcatgggaggatgggacaaggacaaggaaacagactacagagcaaccatcttgagatatggatcaaagtactacttggccatcatggacaagaaatatgcaaaatgcttgcagaagattgacaaggatgatgtcaatggaaattatgagaagataaactacaagctgctgccaggaccaaacaagatgctgccaaaggtgttcttcagcaagaaatggatggcatactacaacccatcagaagacatccagaagatctacaagaatggcaccttcaagaaaggagacatgttcaacctcaatgattgccacaagctgattgatttcttcaaggacagcatcagcagatacccaaaatggagcaatgcatatgatttcaacttctcagaaacagagaagtacaaggacattgctggcttctacagagaggtggaggagcaaggatacaaggtgagctttgaatcagcaagcaagaaggaggtggacaagctggtggaagaaggaaagctctacatgttccagatctacaacaaggacttctcagacaaatctcatggcacaccaaacctccacaccatgtacttcaagctgctgtttgatgagaacaatcatggccagatcaggctctctggaggagcagagctcttcatgagaagagcaagcctcaagaaggaggagctggtggtgcatccagcaaattcaccaattgccaacaagaatccagacaaccccaagaagacaacaacattgagctatgatgtgtacaaggacaagaggttctcagaagatcaatatgagctgcacatccccatcgccatcaacaaatgcccaaagaacatcttcaagatcaacacagaggtgagggtgctgctgaagcatgatgacaacccatatgtcatcggcattgacagaggagaaagaaatctgctctacattgtggtggtggatggcaagggcaacattgtggagcaatattcattgaatgagatcatcaacaacttcaatggcatcaggatcaagacagattatcattcattgctggacaagaaggagaaggagagatttgaagcaaggcagaactggacaagcattgagaacatcaaggagctgaaggctggatacatcagccaagtggtgcacaagatctgcgagctggtggagaaatatgatgctgtcattgctctggaagatctcaacagctactgctcatcagtgtaccagaagtttgagaagatgctgatcgacaagctcaactacatggtggacaagaagagcaacccatgtgcaacaggaggagctctcaagggctaccagatcaccaacaagtttgagagcttcaagagcatgagcacacaaaatggcttcatcttctacattccagcatggctgacaagcaagattgatccatcaacaggatttgtcaacctgctgaagaccaagtacacaagcattgctgattccaagaagttcatcagcagcttcgacaggatcatgtatgttccagaagaagatctctttgaatttgctctggactacaagaacttctcaagaacagatgctgattacatcaagaaatggaagctctacagctatggcaacaggataaggatcttcaggaatccaaagaagaacaatgtgtttgattgggaggaggtgtgcttgacatcagcatacaaggagctcttcaacaaatatggcatcaactaccaacaaggagacatcagagctcttctttgtgaacaatcagacaaggcattctacagcagcttcatggcattgatgagcttgatgctgcagatgaggaacagcatcacaggaagaacagatgtggacttcctcatctcgccggtgaagaacagtgatggcatcttctatgattcaagaaattatgaagctcaagaaaatgccatcctccccaagaatgctgatgcaaatggagcatacaacattgcaaggaaggtgctgtgggccattggacaattcaagaaggcagaagatgagaagctggacaaggtgaagattgccatcagcaacaaggaatggctggaatatgctcaaacatcagtgaagcactaa

SEQ ID NO: 93 - DNA, 3663 bp, synthetic construct
  source 1..3663: mol_type = other DNA; note = LbCas12a RuvC L-del1, codon optimized for Triticum aestivum
atgtccaagctggagaagttcaccaactgctacagcctctccaagaccctccgcttcaaggccatccccgtcggcaagacccaggagaacatcgacaacaagcgcctactggtggaggatgagaagcgcgccgaggactacaagggcgtcaagaagctcctcgaccggtactacctgagcttcatcaacgacgtcctccactccatcaagctcaagaacctcaacaactacatctcactcttccgcaagaagacccggactgagaaggagaacaaggagctggagaacctggagatcaacctgaggaaggagatcgccaaggccttcaagggcaacgagggctacaagagcctcttcaagaaggacatcatcgagacgatcctccccgagttcctcgacgacaaggacgagatcgccctggtgaactcattcaacggcttcaccaccgccttcaccggcttcttcgacaaccgtgagaacatgttctccgaggaggccaagagcacctccatcgccttccgctgcatcaacgagaacctgacccgctacatctcaaacatggacatcttcgagaaggtggacgccatcttcgacaagcacgaggtccaggagatcaaggagaagatcctcaactctgactacgacgtggaggacttcttcgagggcgagttcttcaacttcgtcctgacccaggagggcattgacgtctacaacgccatcatcggcggcttcgtcaccgagtcaggcgagaagatcaagggcctcaacgagtacatcaacctctacaaccagaagaccaagcagaagctccccaagttcaagcccctctacaagcaggtgctctccgaccgggagagcctgagcttctacggtgagggctacacctccgacgaggaggtgctggaggtcttccgcaacaccctcaacaagaactccgagatcttctcaagcatcaagaagctggagaagctcttcaagaacttcgacgagtactcctccgccggcatcttcgtgaagaacggcccggccatctcaaccatctccaaggacatcttcggcgagtggaacgtcatccgtgacaagtggaacgccgagtacgacgacatccacctcaagaagaaggccgtggtgaccgagaagtacgaggacgaccgccgcaagagcttcaagaagatcggcagcttctcacttgagcagctccaggagtacgccgacgccgacctctccgtggtggagaagctgaaggagatcatcatccagaaggtggacgagatctacaaggtgtacggcagctccgagaagctcttcgacgccgacttcgtcctggagaagagcctcaagaagaatgacgccgtggtggccatcatgaaggacctcctcgactcagtcaagagcttcgagaactacatcaaggccttcttcggcgagggcaaggagacgaaccgggatgagagcttctacggcgacttcgtcctcgcctacgacatcctcctcaaggtggaccacatctacgacgccatccgcaactacgtgacccagaagccctactcaaaggacaagttcaagctctacttccagaacccccagttcatgggcggctgggacaaggacaaggagactgactaccgcgccaccatcctccgctacggctcaaagtactacctcgccatcatggacaagaagtacgccaagtgcctccagaagatcgacaaggatgatgtgaacggcaactacgagaagatcaactacaagctcctccccggccccaacaagatgctccccaaggtcttcttctcaaagaagtggatggcctactacaacccctccgaggacatccagaagatctacaagaacggcaccttcaagaagggtgacatgttcaacctcaacgactgccacaagctgatcgacttcttcaaggacagcatctcacggtaccccaagtggagcaacgcctacgacttcaacttctctgagacggagaagtacaaggacatcgccggcttctaccgggaggtggaggagcagggctacaaggtgagcttcgagtccgcctccaagaaggaggtggacaagctggtggaggagggcaagctctacatgttccagatctacaacaaggacttctctgacaagagccacggcacccccaacctacacaccatgtacttcaagctcctcttcgacgagaacaaccacggccagatccgcctctccggcggcgccgagctcttcatgaggcgggcctccctcaagaaggaggagctggtggttcacccggccaacagccccatcgccaacaagaaccctgacaaccccaagaagaccaccaccctctcctacgacgtctacaaggacaagcgcttctcagaggaccagtacgagctccacatccccatcgccatcaacaagtgccccaagaacatcttcaagatcaacaccgaggtgagggtgctactcaagcacgacgacaacccctacgtcatcggcatcgaccgcggtgagcgcaacctcctctacatcgtggtggtggacggcaagggcaacatcgtggagcagtacagcctgaacgagatcatcaacaacttcaacggcatccgcatcaagaccgactaccactcactccttgacaagaaggagaaggagcgcttcgaggcccggcaaaattggacaagcatcgagaacatcaaggagctgaaggccggctacatctcccaggtggtgcacaagatctgcgagctggtggagaagtacgacgccgtcatcgccctggaggacctcaacagctactgctcaagcgtctaccagaagtttgagaagatgctcatcgacaagctgaactacatggtggacaagaagagcaacccctgcgccaccggcggcgccctcaagggctaccagatcaccaacaagttcgagagcttcaagtcaatgtccacccagaacggcttcatcttctacatcccggcctggctcacctccaagattgacccctccaccggcttcgtcaacctcctcaagaccaagtacacaagcatcgccgacagcaagaagttcataagctccttcgaccgcatcatgtacgtgccggaggaggacctcttcgagttcgcccttgactacaagaacttctcacgcaccgacgccgactacatcaagaagtggaagctctacagctacggcaaccgcatccgcatcttccgcaaccccaagaagaacaacgtcttcgactgggaggaggtgtgcctgacctccgcctacaaggagctcttcaacaagtacggcatcaactaccagcagggtgacatccgcgccctcctatgcgagcagagcgacaaggccttctactcctccttcatggccctcatgagcctcatgctccagatgcgcaacagcatcaccggccgcaccgacgtcgacttcctcatctcccccgtcaagaactctgacggcatcttctacgactcacgcaactacgaggcccaggagaacgccatcctccccaagaacgccgacgccaacggcgcctacaacatagcccgcaaggtgctctgggccatcggccagttcaagaaggccgaggacgagaagctggacaaggtcaagatcgccatctccaacaaggagtggctggagtacgcccagacctccgtcaagcactaa

SEQ ID NO: 94 - DNA, 3663 bp, synthetic construct
  source 1..3663: mol_type = other DNA; note = RuvC L-del1, codon optimized for Homo sapiens
atgtccaaactagagaagttcacgaactgctattcgttgagtaaaactttgcgctttaaggcaatccctgtaggtaagactcaggaaaatatcgacaacaagcgactgctggtggaagacgagaaaagggcggaggattacaaaggagtgaaaaagctgttagatcgatactacctgagcttcattaacgacgtgttgcattccatcaaactaaagaaccttaacaattacattagccttttccgcaaaaagacccgtaccgagaaggagaacaaagaattagaaaatctcgagatcaacctgaggaaggagatagcgaaagcgttcaagggcaatgagggctacaagagtctgttcaagaaggacatcatcgaaacgatcctgcctgaatttctggacgataaagatgaaatcgccctcgtaaacagcttcaatggtttcactacggccttcacaggcttttttgacaatcgcgagaatatgttctccgaggaggcgaagtctacaagtattgctttccgctgcattaacgagaatctgacaaggtacatctcaaatatggacatattcgaaaaggtcgatgcgatcttcgacaagcacgaggtgcaggaaattaaggaaaagatcttgaatagcgactatgacgtggaagattttttcgaaggcgagtttttcaacttcgttctgactcaggaaggcattgatgtgtataacgccattattggaggctttgttaccgaaagtggagagaaaatcaagggcctgaacgaatatatcaatctgtataaccaaaaaaccaagcaaaaactccctaaatttaagcccttatataaacaggtgctgagtgatcgggaatcactgtctttttatggggaaggctacacaagcgatgaggaagtgctggaagtcttccgcaacacgctcaacaaaaattccgaaatattttcctcaatcaagaagctggagaaactgttcaagaattttgacgaatattccagcgctggtatttttgtgaagaatgggccagccatcagtactatttcgaaggatatattcggggaatggaacgtcatccgggacaaatggaacgctgagtacgatgacattcatctgaagaaaaaggcagttgtgactgagaagtacgaggatgaccgccgaaagtctttcaagaagatcgggtccttttctctggaacagcttcaggaatacgccgacgccgatctaagcgttgtggagaagcttaaggagatcattatacagaaggttgacgagatctacaaggtctatggctcgagcgagaaacttttcgatgccgatttcgtattggaaaaatccttgaaaaaaaatgatgctgtggtggccatcatgaaagatctgctggatagcgtgaaatctttcgaaaactacatcaaggccttcttcggcgagggtaaagagactaaccgtgacgagtcattttacggcgattttgtgctggcgtatgatattctgcttaaagttgaccacatctacgacgcgataaggaattacgtgacacagaagccatatagcaaggacaagttcaagctctatttccagaacccccagtttatgggcggctgggacaaggacaaggaaaccgattacagagcaacaattctaagatacggttcaaaatactaccttgccataatggacaagaagtatgcgaaatgcctgcagaaaatagacaaagatgacgtaaatggcaactacgagaaaatcaactataaactgctgccgggcccgaataagatgctgcctaaggtgttcttcagcaagaaatggatggcctactataaccccagcgaagatattcaaaagatatataagaatgggacatttaagaagggcgatatgtttaatttaaatgactgccacaagcttatagatttttttaaagacagcatcagtaggtaccccaagtggtcaaatgcgtacgatttcaatttttctgagacggaaaaatacaaagacatcgcaggcttttatcgtgaggtcgaagaacaagggtataaggtttccttcgaaagcgccagcaagaaagaagtggacaaactcgtggaagaggggaaattgtatatgttccaaatttataacaaggacttctctgataaatctcacggtactccaaatcttcatactatgtatttcaaactgctctttgatgagaataatcacggccagatccgcctgtccggcggggctgaactcttcatgcgtagagcctccctgaaaaaggaagagctggttgtccacccggccaattcccccatcgcaaacaagaacccggacaatcccaaaaaaactactacattatcatacgatgtttacaaagataagcgttttagcgaagatcaatatgagctgcacatccctattgcgataaacaagtgtcctaagaacatttttaagattaataccgaggtcagggtgctcttaaagcacgacgataatccttatgtcatcggcatcgacagaggagagcgcaatcttctgtatatagtggtggtggatggaaaaggcaatatcgtggagcaatacagtctgaacgaaattattaacaacttcaatggaatacgaattaagactgactatcactccctgcttgataagaaggaaaaggagagatttgaggctaggcagaattggacttccatagaaaacatcaaagaattaaaggcggggtacatttcacaggtcgtgcataagatttgcgaactcgtagaaaagtacgatgcggtgattgccctggaggatctcaactcctactgctcatctgtttaccagaaattcgagaagatgctcatcgataagctgaactacatggtggacaaaaagagtaacccctgcgcaacaggtggcgctctgaaggggtaccagatcacaaataagttcgagtctttcaagagcatgagcacccaaaacgggttcatcttctacattccagcctggctgacttctaaaatcgacccatccaccggctttgtaaacctcctcaagacaaagtacacctccatcgccgacagcaagaaatttattagctctttcgacaggattatgtacgtccctgaggaggacctgtttgaatttgctttggattataagaatttctctcgtactgatgcagattatatcaagaagtggaagctttacagttacggaaaccgaattcggatttttcgaaacccaaagaaaaacaatgttttcgactgggaggaagtgtgcttgacaagtgcctataaggagctattcaataaatacggcatcaactaccaacaaggagacataagagctcttctctgtgagcaatctgataaagccttctacagctctttcatggcactgatgtctctcatgctccagatgaggaattcaatcaccggtaggaccgatgtggatttcttgatcagcccagtgaagaacagcgacggtatcttttacgattcccgtaattatgaagcccaggagaatgccattctgcccaagaacgcagatgcaaatggtgcttacaatatcgcacgcaaggtgttgtgggccataggccagtttaagaaggcagaagacgaaaaactggataaggtgaaaattgcaatctctaacaaggagtggctcgagtatgcacagacctccgtgaagcactag

SEQ ID NO: 95 - DNA, 3663 bp, synthetic construct
  source 1..3663: mol_type = other DNA; note = LbCas12a RuvC L-del1, codon optimized for Rattus norvegicus
atgtcaaagttggagaagtttacaaattgttactctctcagtaagactcttagattcaaggctatccccgtggggaagacgcaggagaacatcgacaacaaaagattattggtcgaagacgaaaagcgtgctgaagactataagggggtgaagaagttgcttgaccgctattacttgtcattcatcaacgatgtcctgcattccatcaaactaaagaatctgaacaattacatttctttattccgcaaaaagactcggaccgagaaggagaataaagaattagaaaacctagagattaacctcaggaaggagatcgcgaaggctttcaaggggaatgagggttataaaagtctctttaagaaggatatcatcgagaccatcctgcctgaattcctggacgacaaggatgaaatcgcccttgttaacagtttcaatggattcaccaccgcgttcacgggattctttgataatcgcgagaatatgtttagcgaggaggcaaaaagtacttcaattgccttcaggtgcatcaatgagaatttaaccagatacataagcaacatggatatttttgaaaaagtagatgcgatcttcgacaagcatgaggtgcaggagattaaggagaagatcctcaattccgattacgatgtggaggacttcttcgaaggggaattctttaacttcgtgctgacccaagaaggtattgatgtgtacaacgcaatcattggaggattcgtgacagaatccggcgagaagatcaagggtctaaatgaatacatcaatctctacaaccagaagactaagcagaagctgcccaagttcaagccgttatacaaacaggtgctctcagatagggaaagcctgagcttctatggcgagggctacaccagtgacgaggaagtgctcgaggtctttcgcaacaccctcaataagaactcagagatcttctcatccatcaagaagttggagaagctgttcaaaaactttgatgagtatagttctgccgggatctttgtgaaaaacggaccagcaatatcaacaatttccaaagacatttttggagagtggaacgtgatacgtgataagtggaacgcggagtacgatgacatccatctgaaaaaaaaggcagtggtcacagaaaagtacgaagatgaccggcggaagagcttcaagaagatcggcagtttttcgctcgaacaacttcaggagtatgcggacgccgacctttctgtagtcgagaaacttaaagaaatcatcatccaaaaggtcgacgagatttataaagtgtatgggtcgagcgagaagctgttcgatgcggacttcgtcttagaaaagagtctcaagaagaatgacgctgtagtggcaatcatgaaggacctcctagattcagtgaagtcattcgagaattacatcaaggcgttcttcggtgaaggaaaggaaactaaccgcgatgagagtttctatggtgacttcgttctggcttacgacatcctcctaaaggtagaccacatttacgacgcaatacgcaactatgtgacacagaaaccatacagtaaggataaatttaagctttactttcagaatccacagtttatgggcggttgggacaaagataaagagacagactaccgggccaccatcctgagatacgggtccaaatactatctggctattatggataagaaatatgccaagtgtctgcagaagattgacaaagatgacgtaaacggaaactatgagaagataaattacaagctcctccccggacctaataaaatgcttccaaaagtttttttttcaaaaaagtggatggcctactacaacccctctgaggacatccagaagatttacaagaacggcacattcaaaaagggagacatgttcaacctcaacgattgccacaagcttattgacttcttcaaggacagtatctcacggtacccaaagtggtctaacgcttacgactttaatttctcagagactgaaaagtacaaagacatcgctggattctatagagaggtcgaagagcagggctataaagtgagcttcgagtcagcctctaaaaaggaggtcgataaactggtggaagagggaaagctgtatatgtttcagatttataacaaggatttttctgataagagccatggcacgccgaacctccacacaatgtacttcaaattgctcttcgacgagaacaatcacggccagatacgcttgtcaggcggcgccgaactgtttatgagaagggcaagtcttaagaaggaggagctggttgtccacccggccaacagccccatcgcaaacaagaaccctgataacccaaaaaaaaccacaaccctttcatatgacgtgtataaggataagcggttctcagaagaccagtatgagcttcacattcctatcgcaattaacaagtgtcctaaaaacatcttcaaaatcaatacagaagtaagagtgctgctgaaacacgatgacaatccttacgtgattggaattgacagaggggaacgtaatctcctgtacattgtggtcgtggatggcaaaggaaacatagtggagcaatacagcctgaacgaaatcattaacaattttaatggaattcggatcaaaaccgattaccattccctccttgacaagaaggaaaaggaaagattcgaggccagacaaaattggacatcaatcgaaaacatcaaagagcttaaggcggggtacatcagccaggtggtgcacaagatatgtgaactggtagagaagtacgatgccgtcattgcactggaagatctgaatagttactgctccagtgtttatcagaagttcgagaaaatgctaatcgacaagctgaattacatggtcgacaagaagtcaaacccctgtgcaactggcggggcactgaaggggtaccaaattactaacaaatttgaatcttttaagtcaatgtcaacccagaacggcttcatcttttatattccggcatggcttacttcaaagatcgatccttcaacaggctttgtgaacctgctcaagactaaatatacaagcatcgcagattctaagaagtttatctcttcctttgacaggattatgtacgtgccggaagaggatctcttcgaatttgccctcgactacaaaaacttttccaggaccgatgccgattatattaagaaatggaaattgtactcatatggaaatcgcatccgtatttttaggaatcctaaaaagaacaatgtctttgactgggaggaggtctgtctgaccagcgcttataaggaactgtttaataagtatgggataaattatcagcagggcgacattagggcgctgttatgcgagcagtccgataaggccttttattcttctttcatggcactcatgagtttaatgctccagatgcgcaactctatcacaggcagaaccgatgtggacttcctgatcagtcccgtcaaaaattctgatggcattttttacgacagtagaaattacgaggcacaggagaacgctatcctccccaagaatgccgacgcaaatggcgcatacaacatcgccaggaaagtcctgtgggctatcggacagtttaaaaaggctgaggacgaaaaattggacaaggtaaaaattgctatttcaaacaaggagtggctggaatatgcgcagacctcggtgaagcactag

SEQ ID NO: 96 - DNA, 3663 bp, synthetic construct
  source 1..3663: mol_type = other DNA; note = LbCas12a RuvC L-del1, codon optimized for Bos taurus
atgagcaagctggagaaatttactaattgttactctttatctaaaacgttaaggttcaaggccattcctgtcggaaagacccaggagaacatcgacaacaaacgcctgcttgtggaagacgaaaaaagggcagaggactataagggtgtgaaaaaacttcttgacaggtactatttgtcctttattaacgacgtgctccatagcataaagctaaagaatctgaataattatatcagtctctttcgcaagaaaaccaggaccgagaaggaaaataaagaattggagaatctggaaataaacttgcgaaaggaaatcgccaaagcgtttaaagggaacgagggttacaagagcttgttcaagaaggatattatagaaaccatactcccagagtttctggacgacaaggacgagattgccctcgtcaactcctttaacggatttacgaccgccttcactggattttttgacaaccgcgagaacatgttctcagaagaggcaaagtcgacatcgatcgcgtttaggtgcatcaacgagaatctgactcggtacataagtaacatggacatcttcgaaaaggtagatgcaatctttgacaagcacgaagttcaagagatcaaagagaagatccttaactcagattacgatgtcgaggatttttttgagggagagttttttaattttgtgctcacacaggaaggcatcgatgtttacaacgcaatcatagggggattcgtgacggagtctggcgaaaaaatcaagggacttaacgagtacatcaacctatacaatcagaagacaaaacaaaagctcccaaagtttaagcctctgtacaaacaagtcttgtcagatagagaaagcctgtcattttacggagaaggctacacatcagatgaagaagtgctggaggtgtttcgcaatactctgaataaaaactctgagattttcagtagcattaagaaactggaaaagctgttcaaaaacttcgacgagtactcctccgcaggcattttcgtcaaaaatggaccagcaatttcaacaatcagcaaggatattttcggggaatggaacgtcattcgggacaagtggaatgccgagtatgacgacatccatctgaagaagaaggcagtggtgaccgagaaatatgaggacgataggagaaaaagcttcaagaagataggtagcttctcactagagcagttacaggagtacgcggacgctgacctatctgtggtcgaaaagctgaaggagattattatccagaaggtcgatgagatctataaggtgtatgggtcgtctgagaaattgttcgacgcggacttcgtgctcgagaagtcgctaaagaagaacgatgcagtcgtggcgattatgaaggacctgcttgacagcgtgaagagcttcgagaactatattaaggccttcttcggtgagggaaaggagactaatagggacgagtcgttctacggcgactttgtcttggcgtacgacatccttttgaaagtagatcacatttacgacgccatcagaaactatgttacccagaagccttacagtaaggacaaattcaagttgtattttcagaatccccagttcatggggggatgggataaagacaaagagaccgactacagagccacaatcttgcgatatggctccaaatattacctggccatcatggacaagaaatatgccaaatgtcttcagaaaatcgataaggacgacgttaatggcaactatgagaagattaactacaagcttctgcctgggccaaacaagatgctgccgaaagtgttcttctcgaagaagtggatggcgtactataatccttccgaagacattcaaaaaatctacaagaatgggactttcaagaaaggagacatgttcaatctgaatgactgccacaaattgattgatttttttaaagattctatctctagatatcctaagtggtccaatgcatatgacttcaatttctccgaaaccgagaagtacaaggacattgccgggttttatcgagaggtggaagaacagggctataaagtgagcttcgaaagcgcctcaaaaaaggaagtcgataagctggttgaagaggggaaactgtacatgttccagatttacaataaagactttagtgacaaatcacacggtacaccaaacctacacacgatgtattttaaacttttgtttgacgagaataaccacggccagatcagactgagtgggggagctgagctgttcatgcgccgggcaagcctgaagaaggaagagctggttgttcaccctgccaactcaccaatagctaataaaaatcctgataaccccaaaaagaccacaactctcagttacgatgtgtacaaggataaacggttttccgaagaccagtacgaactccacatccctatagcaataaataaatgccccaaaaatatattcaaaatcaacacggaggtgcgggtgttactgaagcacgatgacaacccctacgtaatcggaatagaccgcggagaacggaacctactctacatcgttgtggttgacgggaagggcaatatcgtggagcagtacagtctcaacgaaatcatcaataactttaacggcatccgtataaagaccgactaccatagccttctggacaagaaagagaaggagcggtttgaggctcggcagaattggacctccattgaaaacatcaaggagctaaaggcgggctatatatctcaggttgtgcataagatctgcgagcttgtggagaaatacgatgccgtgatcgccttagaagaccttaattcatactgttcttcagtgtaccagaagttcgaaaaaatgctgatcgacaaactgaattacatggtggacaaaaaatctaacccttgtgccacaggcggagcgctcaagggataccagattacaaataaattcgaatcatttaaatccatgtccactcagaacggattcatcttttacatccctgcttggctgactagcaaaatcgatccatctactgggttcgtgaaccttctgaaaacaaagtatacaagcatcgcagatagtaagaaattcattagctccttcgatcgcatcatgtatgtcccagaggaagacctgttcgagttcgcgttggactacaaaaacttctcgcggacagacgcggattacatcaagaaatggaagctgtactcctacggcaatcggatcaggatattccgcaatcccaaaaaaaataatgtcttcgactgggaagaggtctgtctgacatcagcttataaggaactgttcaacaagtacggcatcaattatcagcagggggacatccgggccttattatgcgagcagtcggacaaggcgttttatagttcattcatggccctgatgtccctgatgctgcagatgcgcaacagcataactggccggaccgatgtggacttcctgattagccccgtgaagaattcagatggaatattctatgattcccgaaactacgaggcgcaggaaaacgccatcctgcccaagaacgctgacgctaatggcgcttacaacatcgcaaggaaggtcttgtgggcaattggacagtttaagaaagccgaggacgagaagctcgacaaagtgaagatagccatctccaacaaagaatggctggagtacgctcaaacttcggtgaaacactag

SEQ ID NO: 97 - DNA, 3663 bp, synthetic construct
  source 1..3663: mol_type = other DNA; note = LbCas12a RuvC L-del1, codon optimized for Mus musculus
atgtccaagttggaaaaattcaccaattgctatagcctgtccaagaccttgaggtttaaagctatacctgtgggtaaaacacaagagaacatagacaacaagcgtttgctggtggaggacgaaaagagagcggaggactataagggtgtgaaaaagcttcttgacagatactacctatcttttatcaatgatgtgctgcacagcatcaaacttaagaatctgaacaattatatttccctgttcagaaaaaagactagaacggaaaaggagaacaaagagctcgagaatctggaaataaacctgcgtaaagagatagctaaggccttcaagggcaatgaaggatacaaatccctgttcaagaaagacattattgaaactatcctccccgaatttttagatgataaggacgagatcgccctggtcaacagcttcaatggcttcacaaccgcttttacaggcttcttcgacaaccgggagaatatgttttcagaggaggctaagagtactagcatcgcatttcgttgtatcaacgaaaacctaacgcggtatataagcaacatggatatatttgaaaaggtggatgctatcttcgataagcacgaggtgcaggaaatcaaagagaagatcctgaatagcgactacgacgtcgaggacttttttgagggcgaattttttaacttcgttttgacccaggaaggaatcgatgtgtacaacgctatcatcggtgggttcgttactgagtctggagaaaagatcaagggactgaatgaatacatcaacctctataatcagaagacaaagcagaaactccctaagtttaaacccctttacaagcaggtgctctccgaccgggagagcctgtcattctatggagagggctacacatctgatgaagaagtgctcgaggtgtttagaaacacactcaataagaatagcgagatcttcagttcgattaagaagttggagaaactgttcaagaactttgacgaatacagcagcgctggaattttcgtcaagaatggtcctgctatcagcactatctccaaagacatctttggggaatggaacgttatccgggacaagtggaatgctgaatatgacgatatccacctgaaaaagaaggctgtcgttacagagaagtacgaagacgaccgccgcaagtcattcaaaaaaatcgggtcttttagcctagaacagcttcaggagtatgcagatgcagatttaagcgttgtcgagaagctgaaggaaatcatcatccaaaaggtcgacgaaatatataaagtttatggttctagcgagaagctcttcgacgcagattttgtgctcgagaagtcattaaagaagaatgatgctgtggtagctataatgaaggaccttctggatagcgtcaaaagcttcgagaactacatcaaggcctttttcggagaaggcaaggagactaaccgggatgaaagtttctacggggattttgtactcgcttatgacattctgctgaaagtcgaccatatctacgatgctatccgaaactacgtaacccaaaagccttactcaaaggataaattcaaactctactttcagaacccacagttcatggggggctgggataaagacaaagaaacggattatcgcgcaaccatcctgaggtatggtagcaagtactatctggccattatggacaagaagtatgctaaatgccttcagaagattgataaggacgacgtaaacggtaattacgaaaagattaactacaagttgcttcctggacccaataagatgcttccaaaggtttttttttctaaaaagtggatggcctattacaatcccagcgaagacatccaaaagatctataagaacggcacatttaagaagggagacatgtttaatttgaatgattgtcataagctaatagattttttcaaggactcaatcagccgctacccaaaatggagcaacgcttatgacttcaattttagcgagaccgaaaaatataaagacattgccggtttctatcgagaggtggaggaacagggctataaggtgagcttcgagtcagctagtaagaaagaggttgataaactggtggaggaggggaagctttacatgttccagatctacaacaaggatttcagcgataagtcccatggcacaccaaatctgcacacaatgtactttaagctgcttttcgacgagaataatcatgggcagattagactgagtggtggcgctgaattgtttatgcgacgtgctagtctgaaaaaagaagaactggttgtgcaccccgccaattctcctatcgccaacaagaatcccgacaacccaaaaaaaactaccactctgagctacgacgtttacaaggacaagagattttctgaagaccagtacgaattgcacatccctatcgccatcaacaaatgcccaaaaaatatcttcaagataaacaccgaggttcgggtgctgctgaaacatgacgacaacccttatgtcatcgggatagaccggggcgaacggaacctgctctatattgtggtggtcgatgggaaaggtaatatcgtcgaacaatatagtctgaacgagattataaataactttaatggaatcaggattaaaacagactaccactccctgttggacaagaaggagaaagagcgctttgaagcccgtcaaaattggacctcgattgagaatattaaagagcttaaggctggttacatttcacaggtcgttcataagatttgcgagctggtggagaagtatgacgccgtgatcgctttagaggacctcaattcctactgttcctccgtgtaccagaaatttgagaagatgttaattgacaaacttaattacatggtcgacaagaagtcaaatccctgcgccaccggtggagccctgaaggggtaccagatcacaaacaagtttgagtcttttaaatccatgagtactcaaaacggctttatcttctacatcccggcctggctgacaagtaagatcgatccgtccaccggctttgttaacttactaaagaccaaatatacatccatcgccgattctaaaaaatttatatctagcttcgatcgcatcatgtacgtgccagaagaggacctcttcgagttcgccctggattacaagaacttttctcggaccgacgccgactacattaaaaaatggaagctgtactcatatggaaaccgaattcgtattttcaggaacccaaaaaaaaacaacgtgtttgactgggaggaggtctgcctcacttccgcatataaagagcttttcaacaagtatggcatcaactatcagcagggcgatatcagagctctactgtgcgagcagagtgacaaggcgttctattcttcattcatggcactaatgagcctgatgttacagatgagaaatagtattaccggcaggacagacgtggacttcctgatctcacccgttaagaacagcgatggaatcttctatgactctcgaaattacgaggcacaggaaaacgccatcctgcccaagaatgcggatgccaacggcgcttataacatcgctcgcaaagtgctgtgggccatcggccaattcaagaaggccgaagatgagaagttggacaaggtgaaaatagccatctctaataaagaatggctggaatacgctcagacatcagtgaagcactag

SEQ ID NO: 98 - DNA, 3663 bp, synthetic construct
  source 1..3663: mol_type = other DNA; note = LbCas12a RuvC L-del1, codon optimized for Sus scrofa
atgagtaagctcgagaaattcacaaactgttactccctcagcaagaccctgcgattcaaagctatccctgtgggcaaaacccaagagaacatcgacaacaagaggctgctggttgaagatgagaagcgcgctgaggactacaagggcgtcaagaagctgctcgacagatactatctctcattcattaacgacgtccttcacagcattaaactaaagaatctgaacaactacatctccctgtttagaaaaaagaccagaaccgaaaaggagaacaaggaactggagaatctcgagattaacctgcgaaaggagattgctaaggcgtttaagggtaacgaaggttacaaaagtctcttcaaaaaggacattattgaaaccattctcccggagttcttggatgacaaggatgagatagccctcgtcaactccttcaatggatttactacagcattcactgggttcttcgataaccgggagaatatgttttccgaggaggctaaaagcacgtcaatcgctttcagatgtatcaacgaaaaccttacaaggtacatatccaacatggacattttcgagaaggtggacgcgatatttgataagcatgaggtccaggagatcaaggagaagatcctcaactccgattacgatgtggaggatttcttcgaaggggagtttttcaatttcgttctaactcaggagggaattgatgtgtacaatgcaatcataggaggattcgtaactgagagcggtgagaagatcaaaggcctgaatgaatacatcaatctgtataaccagaagaccaaacaaaagctccccaaattcaagccactgtataaacaagtgctctcagatagggaatcactttcattctacggcgagggctatacctctgatgaagaagtgctggaggtgtttagaaataccttgaacaaaaacagtgaaatcttctcctctattaagaagctagagaaactgtttaaaaatttcgatgagtactccagtgccggtatcttcgttaaaaatggacccgctatcagcaccatttcaaaggacatattcggcgagtggaatgtcattcgggacaaatggaatgccgagtacgatgacatccacctgaagaagaaggcagtggtcactgagaaatacgaagacgaccggcggaaaagctttaagaaaatcgggagcttctcactggaacaactgcaggaatacgccgatgctgacttaagcgttgtcgaaaagcttaaagagattatcatccaaaaggtcgacgagatctataaggtctatggctcctccgagaagctgtttgacgccgatttcgtgttggagaagtccctgaagaagaatgacgctgtcgttgccatcatgaaggacctgttggattctgtcaaaagttttgaaaactacataaaagctttcttcggcgagggtaaggaaacgaatagggacgagagcttctacggagacttcgttctggcctatgatattttgctgaaagtggaccacatctacgacgccatccgcaactatgtcacccagaagccgtacagcaaagacaagtttaaattatacttccaaaacccccaatttatgggagggtgggacaaggacaaggagaccgactaccgcgctacaatcttgcggtacggctcgaagtactacctcgccataatggataagaagtacgcaaaatgtctgcagaaaatcgacaaggatgacgtcaacggtaactatgagaagataaactacaagctcctgccgggccccaataagatgttgcccaaagtgttcttctccaagaagtggatggcctactataatccatcagaagatatccaaaaaatctataaaaatggcaccttcaagaaaggcgacatgttcaacttgaatgactgccataagctcatcgattttttcaaggattctatcagtcgatacccgaaatggagcaacgcctacgactttaactttagcgaaaccgaaaagtacaaggatatcgccggattctaccgcgaggtcgaggagcagggatacaaagtgtcatttgagagcgcctccaaaaaggaagttgacaaattggtcgaggaggggaagttatatatgtttcagatctacaacaaggacttttctgacaagagtcacggcacccctaacctgcacaccatgtattttaaattactgttcgacgagaacaaccacggacagatcagactctcgggaggggctgagctgttcatgcggcgtgcctctttgaaaaaggaggaactggtcgttcacccggccaactcaccaatcgcaaacaaaaatccagataacccgaaaaaaaccacgaccctgagctacgacgtgtacaaagacaagcgattttctgaagatcaatatgaactgcacatcccaatcgctatcaataaatgtccgaagaacatcttcaaaatcaatacagaagttagggtgctgctcaagcacgacgacaacccgtacgtgatcggaattgatagaggcgagcggaacctgctgtacatcgtggtcgtcgacggtaaaggtaacattgtggaacagtactcactcaacgaaatcataaataacttcaacggcatccgaattaagaccgactaccacagcctgctcgataagaaggaaaaagaacggttcgaggcacggcagaactggacctctatcgagaacataaaggaactgaaggctggatatatcagccaggtcgtgcataaaatttgcgaactcgtcgagaagtacgacgccgttatcgctctggaggacctgaattcctactgctctagtgtctaccagaagttcgaaaaaatgttaatagacaagctgaactatatggtggataagaaaagtaacccctgtgcaaccgggggcgccctcaaaggttaccagatcacaaataaattcgaaagcttcaagagtatgagtacacagaatgggttcatcttttatatcccagcctggctgacttctaagatcgatccttccaccgggtttgttaacttgctgaaaacgaaatataccagcatcgctgactcgaagaagttcatttcctcctttgatcgaatcatgtacgtacccgaagaggacctattcgaatttgctctggactacaagaactttagccggacagatgccgattatatcaagaaatggaagctatacagctacggcaataggattcgcatatttaggaaccccaaaaaaaataacgtgttcgactgggaggaggtatgtctgactagcgcttacaaagagctgttcaacaagtacggcataaactaccaacaaggcgatatacgcgctctgctttgtgagcagagcgacaaagcgttctattcctctttcatggcacttatgtccctgatgctgcagatgcggaactccatcaccggcaggaccgacgtggattttttaatctccccagttaaaaacagcgatggaatcttttacgatagccggaactacgaagctcaagagaacgctatccttccgaagaatgccgacgcgaacggagcctataatattgcccggaaagtgctgtgggcaatagggcagttcaagaaggctgaggatgagaagctggataaagtcaaaattgccatcagtaacaaggagtggctggagtacgcacaaacctctgttaagcactag

SEQ ID NO: 99 - DNA, 3663 bp, synthetic construct
  source 1..3663: mol_type = other DNA; note = LbCas12a RuvC L-del1, codon optimized for Gallus gallus
atgtctaagctggaaaaattcaccaattgttactccctcagtaagaccttgcgcttcaaggccatccctgtgggcaaaacccaggagaatatcgataataagcgcttacttgtggaggacgaaaaaagggccgaagattataagggcgtgaagaaactgttggacagatactacttatcatttatcaacgatgtgttgcactctatcaaactgaagaacctaaacaactacataagcctgttccggaagaaaactcggacagagaaagaaaacaaagagctcgaaaacctggagatcaacctgaggaaggagatcgccaaggccttcaaagggaatgaagggtataagtctcttttcaagaaggatatcatcgagaccatactcccggagtttctggacgataaggacgaaatcgcacttgtgaattcgttcaatggatttacaaccgccttcacaggctttttcgataacagagagaacatgttcagtgaagaggccaagtccaccagcatcgcttttcgttgcataaacgaaaatttgacccgctatatatcaaacatggatatctttgaaaaggtggatgctatctttgacaagcacgaagtgcaggaaatcaaagagaagatcttgaattctgattacgatgtggaggatttcttcgagggggagttcttcaattttgtgctgactcaagaaggcattgacgtttacaacgccattattggaggattcgtgacggagtctggtgagaaaattaaggggttaaacgagtatataaatctgtacaatcagaagactaagcaaaagctgccgaagtttaagcccttgtacaaacaggtcctctctgaccgcgaatcattgtccttttacggcgaagggtacacctccgacgaggaggtgctggaagttttccgcaacacattgaacaagaattccgaaatcttttcatctataaaaaaactggagaaactattcaaaaattttgacgagtacagctccgcaggtattttcgtaaaaaacggcccagccatctccactatcagcaaggacatattcggagagtggaacgtgattagagacaagtggaatgctgagtacgatgatatccacctcaagaaaaaggcagttgttactgagaagtatgaggatgaccggcggaaaagtttcaagaagattggtagctttagtctggagcagctgcaggagtacgcagacgcggatttgagcgtggtggagaagctgaaggaaataattatacagaaggtagatgagatttataaagtgtatggtagctccgagaaattattcgacgctgatttcgtcctggagaagagcctgaagaaaaacgacgcagtggtggcaatcatgaaggatctgctggattctgttaagtcttttgaaaactacataaaggcgttcttcggagagggcaaagaaaccaaccgcgacgagtcattctacggagattttgtgctcgcttacgatatcttgctgaaagtcgatcacatctatgacgccatccgtaattacgtcacgcagaagccctacagtaaggacaagtttaagctttatttccaaaacccccagttcatgggtgggtgggacaaagataaggagaccgattacagggcgactatcctgcggtacggcagtaaatactacctagcaattatggacaaaaagtacgcaaagtgtctccagaagatcgataaggacgatgtgaacggcaactacgaaaaaatcaattataaactgctgcccggccctaacaagatgttgcccaaggtctttttttccaagaagtggatggcgtattataaccccagtgaagatatacagaaaatatacaagaatggaacgtttaagaagggtgatatgtttaatcttaacgattgccataagcttatagactttttcaaagattcaattagtcgataccccaaatggagcaacgcgtacgactttaattttagtgaaaccgaaaaatacaaagatatagcgggcttctacagagaagtcgaagagcagggctataaggtgtcatttgagtcagcttctaagaaggaagttgacaaactggtggaagaaggaaaactgtacatgtttcagatttataacaaagatttcagtgataagtcgcacgggaccccaaacctgcacacaatgtacttcaaactgctgtttgacgagaataaccacgggcaaatccggttgtcaggtggggccgagctttttatgagaagagctagtctcaagaaggaagagctggtggtgcaccccgctaatagcccgatcgctaataaaaacccagacaacccaaagaagaccacaacactgagctacgatgtgtacaaggataaaaggttctcagaggatcaatacgagttacatataccgatcgcaattaataagtgtccaaagaatatctttaagattaatacagaagtccgggtgttgttgaaacatgacgacaatccgtatgtaattggaatagatcgcggagagcgaaacctgctgtacattgtggtggtggatggtaaggggaatattgtggagcagtactccctcaatgaaattattaataattttaacggaattcgtattaagacggactatcactctcttctggacaagaaggaaaaggaacgattcgaggcgcgccaaaactggaccagcatagagaacatcaaagagttaaaggcaggatacatctcacaagtagtgcataaaatctgcgaactggttgaaaagtacgatgcggtaattgcactggaagatctgaactcttactgctcctccgtttaccagaagttcgagaaaatgctgatcgataaactaaactatatggtcgataagaaatcgaacccatgtgcaaccggcggggctctaaaagggtatcagattacaaataaatttgagtcctttaagtccatgtcgacacaaaacggcttcatcttctacataccagcatggttaacttctaagatagacccctcgacgggcttcgtgaatttattgaagaccaagtatacctcaatcgctgatagcaagaaatttatttccagtttcgaccgcataatgtacgttccagaggaggacctattcgagttcgccttggactataagaatttttctaggaccgacgctgactatattaagaaatggaagctgtactcttatggcaacaggatcaggatcttccgtaatcccaagaagaacaacgtatttgactgggaagaggtctgcctgactagcgcttacaaggagctgtttaataaatatgggataaactatcagcaaggcgacatacgggccctgctgtgcgaacagtccgacaaggcattctacagttctttcatggccctgatgagcctgatgctgcagatgagaaactctataacaggaagaacagatgtcgactttcttatcagtcccgtcaagaactctgatggcatcttttacgacagtcgcaattatgaggcgcaggagaatgcgattctgcccaagaacgctgatgcaaacggagcttacaatattgccagaaaagtcctgtgggctataggtcagttcaagaaagccgaagacgaaaaactggacaaggtgaagatcgcaatcagcaacaaggagtggctggaatacgctcagacgagtgtcaaacattag

SEQ ID NO: 100 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSCLRRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 101 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYLGTVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 102 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSPITRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 103 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSIFRRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 104 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSPRSSVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 105 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSYIRRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 106 - protein, 1219 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSTMFRVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 107 - protein, 4 aa, synthetic construct
  source 1..4: note = Sequence of Lid4.1 shown if Fig. 5D
CLRR

SEQ ID NO: 108 - protein, 4 aa, synthetic construct
  source 1..4: note = Sequence of Lid4.2 shown if Fig. 5D
YLGT

SEQ ID NO: 109 - protein, 4 aa, synthetic construct
  source 1..4: note = Sequence of Lid4.3 shown if Fig. 5D
PITR

SEQ ID NO: 110 - protein, 4 aa, synthetic construct
  source 1..4: note = Sequence of Lid4.4 shown if Fig. 5D
IFRR

SEQ ID NO: 111 - protein, 4 aa, synthetic construct
  source 1..4: note = Sequence of Lid4.5 shown if Fig. 5D
PRSS

SEQ ID NO: 112 - protein, 4 aa, synthetic construct
  source 1..4: note = Sequence of Lid4.6 shown if Fig. 5D
YIRR

SEQ ID NO: 113 - protein, 4 aa, synthetic construct
  source 1..4: note = Sequence of Lid4.7 shown if Fig. 5D
TMFR

SEQ ID NO: 114 - DNA, 63 bp, synthetic construct
  source 1..63: mol_type = other DNA
tttcacttgggtgccctaggaagctgcctctggcctatcctgtgcctgaagtcgccatccaaa

SEQ ID NO: 115 - DNA, 20 bp, synthetic construct
  source 1..20: mol_type = other DNA
acttgggtgccctaggaagc

SEQ ID NO: 116 - DNA, 20 bp, synthetic construct
  source 1..20: mol_type = other DNA
gatggcgacttcaggcacag

SEQ ID NO: 117 - DNA, 60 bp, synthetic construct
  source 1..60: mol_type = other DNA
tttaagggggtagcatttctctgtaaactccacagaagtgtgggaggggaagtaagtaaa

SEQ ID NO: 118 - DNA, 20 bp, synthetic construct
  source 1..20: mol_type = other DNA
agggggtagcatttctctgt

SEQ ID NO: 119 - DNA, 20 bp, synthetic construct
  source 1..20: mol_type = other DNA
cttacttcccctcccacact

SEQ ID NO: 120 - DNA, 59 bp, synthetic construct
  source 1..59: mol_type = other DNA
tttagcgctgtcaagaaccagaatgtcttaacattaatagaacagtgaggaccctgaaa

SEQ ID NO: 121 - DNA, 20 bp, synthetic construct
  source 1..20: mol_type = other DNA
gcgctgtcaagaaccagaat

SEQ ID NO: 122 - DNA, 20 bp, synthetic construct
  source 1..20: mol_type = other DNA
agggtcctcactgttctatt

SEQ ID NO: 123 - protein, 114 aa, Acidaminococcus sp.
  source 1..114: note = BV3L6
FDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTS

SEQ ID NO: 124 - protein, 113 aa, Methanomethylophilus alvus
  source 1..113: note = Candidatus Methanomethylophilus alvus
YDYRKALDVREYDNKEARRNWTKVEGIRKMKEGYLSLAVSKLADMIIENNAIIVMEDLNHGFKAGRSKIEKQVYQKFESMLINKLGYMVLKDKSIDQSGGALHGYQLANHVTT

SEQ ID NO: 125 - protein, 113 aa, Eubacterium rectale
YDYQIKLKQQEGARQTARKEWKEIGKIKEIKEGYLSLVIHEISKMVIKYNAIIAMEDLSYGFKKGRFKVERQVYQKFETMLINKLNYLVFKDISITENGGLLKGYQLTYIPEK

SEQ ID NO: 126 - protein, 115 aa, Thiomicrospira sp.
  source 1..115: note = XS5
HVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFES

SEQ ID NO: 127 - protein, 115 aa, Francisella tularensis
  source 1..115: note = subsp. novicida
MKTNYHDKLAAIEKDRDSARKDWKKINNIKEMKEGYLSQVVHEIAKLVIEHNAIVVFEDLNFGFKRGRFKVEKQVYQKLEKMLIEKLNYLVFKDNEFDKTGGVLRAYQLTAPFET

SEQ ID NO: 128 - protein, 115 aa, Francisella tularensis
MKTNYHDKLAAIEKDRDSARKDWKKINNIKEMKEGYLSQVVHEIAKLVIEYNAIVIFEDLNFGFKRGRFKVEKQVYQKLEKMLIEKLNYLVFKDNEFDKTGGVLRAYQLTAPFET

SEQ ID NO: 129 - protein, 115 aa, Moraxella bovoculi
MTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTD

SEQ ID NO: 130 - protein, 115 aa, Moraxella bovoculi
VTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTD

SEQ ID NO: 131 - protein, 115 aa, unidentified
  source 1..115: note = Lachnospiracea bacterium
IKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFES

SEQ ID NO: 132 - protein, 113 aa, Porphyromonas crevioricanis
IDYHDLLESRDKDRQQERRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKS

SEQ ID NO: 133 - protein, 119 aa, Butyrivibrio sp.
  source 1..119: note = NC3005
IETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKS

SEQ ID NO: 134 - protein, 119 aa, unidentified
  source 1..119: note = Lachnospiracea bacterium
IETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKS

SEQ ID NO: 135 - protein, 17 aa, synthetic construct
NSGFKNSRVKVEKQVYQ

SEQ ID NO: 136 - protein, 17 aa, synthetic construct
NSGSINKWRYPEAQVYQ

SEQ ID NO: 137 - protein, 17 aa, synthetic construct
NSGKFSFLVSSSKQVYQ

SEQ ID NO: 138 - protein, 17 aa, synthetic construct
NSGIIMLKHASGCQVYQ

SEQ ID NO: 139 - protein, 17 aa, synthetic construct
NSGYWLHRQFTGRQVYQ

SEQ ID NO: 140 - protein, 17 aa, synthetic construct
NSGFKNKWRYPEAQVYQ

SEQ ID NO: 141 - protein, 17 aa, synthetic construct
NSGFKNFLVSSSKQVYQ

SEQ ID NO: 142 - protein, 17 aa, synthetic construct
NSGLLFWSCNGGLQVYQ

SEQ ID NO: 143 - protein, 17 aa, synthetic construct
NSGFKNWSCNGGLQVYQ

SEQ ID NO: 144 - protein, 17 aa, synthetic construct
NSGKFSFLVSSSKQVYQ

SEQ ID NO: 145 - protein, 9 aa, synthetic construct
NSYCSSVYQ